(19) United States
(12) Patent Application Publication
(10) Pub. No.: US 2007/0106074 A1
(43) Pub. Date: May 10, 2007

(54) Iron catalyzed cross-coupling reactions of imidoyl derivatives

(76) Inventors: Roger Olsson, Bunkeflostrand (SE); Fredrik Ek, Lund (SE); Lars K. Ottesen, Valby (DK)

Correspondence Address: KNOBBE MARTENS OLSON & BEAR LLP, 2040 MAIN STREET, FOURTEENTH FLOOR, IRVINE, CA, 92614, US

(21) Appl. No.: 11/582,729

(22) Filed: Oct. 17, 2006

Related U.S. Application Data
(60) Provisional application No. 60/727,971, filed on Oct. 17, 2005.
(60) Provisional application No. 60/727,604, filed on Oct. 18, 2005.

(51) Int. Cl. C07D 267/02; C07D 243/10; C07F 7/02

(52) U.S. Cl. 540/547; 540/557; 546/14

(57) ABSTRACT

Disclosed is a process for preparing a compound of formula A-N═C(D)(B), from a compound of formula A-N═C(E)(B) and a compound of formula D-M using an iron catalyst, where the process has is represented by Equation (I)

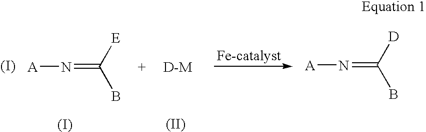

This application claims priority to U.S. Provisional Patent Application Ser. Nos. 60/727,971, entitled IRON CATALYZED CROSS-COUPLING REACTIONS OF IMIDOYL DERIVATIVES, filed Oct. 17, 2005; and 60/727,604, entitled IRON CATALYZED CROSS-COUPLING REACTIONS OF IMIDOYL DERIVATIVES, filed Oct. 18, 2005; which are all incorporated by reference herein in their entireties, including any drawings.

### BACKGROUND OF THE INVENTION

1. Field of the Invention

This invention relates to the fields of organic chemistry, pharmaceutical chemistry, fine chemicals and material chemistry. In particular it relates to the cross coupling of imidoyl halides, sulfonates, and phosphates with organometallic reagents in the presence of iron complexes as the catalysts or pre-catalysts.

2. Description of the Related Art

Iron is one of the most abundant metals on earth, and one of the most inexpensive and environmentally benign in sharp contrast to other metals, such as palladium or nickel, commonly used as catalysts in cross coupling reactions. Despite its advantages, it is surprising that, until recently, iron was relatively underrepresented in the field of catalysis compared to other transition metals and only few examples are known, where iron reagents catalyze cross coupling reactions (Fürstner, A. and Martin, R.; Chemistry Lett. 2005, 34, 624-629).

Iron salts (e.g. iron (II, III) chlorides) were first reported, in 1971, by Kochi et al. (Tumura, M.; and Kochi, J. K.; J. Am. Chem. Soc. 1971, 93, 1487) to be effective catalysts between the coupling of alkyl and aryl Grignard reagents with alkyl and alkenyl halides. However, iron-catalyzed cross coupling of aryl Grignard reagents is more sensitive to the chosen electrophile due to the competing homo-coupling. Cross coupling between two aryl moieties stills remains problematic owing to the extensive formation of biaryls (Cahiez, G. and Marquais, S.; Pure Appl. Chem. 1996, 68, 53-60). From the middle of the 1990s, due to pioneering work by Fürstner and Cahiez, attention returned to the field of iron-catalysts in cross coupling reactions. Cahiez and co-workers reinvestigated the iron-catalyzed alkenylation of Kochi et al. and presented a way to increase the yields in these reactions by addition of NMP.

In 2002 Fürstner et al. greatly increased the scope of iron-catalyzed cross coupling reactions with organometallic reagents by introducing aryl and hetero aryl chlorides, tosylates and triflates as suitable electrophiles (Fürstner, A.; Leitner, A.; Méndez, M. and Krause, H.; J. Am. Chem. Soc. 2002, 13856-13863).

Knochel and co-workers introduced the iron-catalyzed aryl-aryl cross coupling reactions with magnesium-derived copper reagents, thereby considerably decreasing the amount of homocoupling (Sapountzis, I.; Lin, W.; Kofink, C.; Despotopoulou, C. and Knochel, P.; Angew. Chem. 2005, 44, 1654-1657).

In 2004, work by Hayashi et al. (Nagano, T. and Hayashi, T. Organic Letters 2004, 6, 1297-1299). demonstrated a lower reactivity of aryl triflates towards iron-catalyzed cross coupling compared to the alkyl halide. Hocek and Dvovráková have likewise successfully applied iron salts in the monomethylation reaction of 2,6-dichloropurines with MeMgCl. (Hocek, M. and Dvovráková, H.; J. Org. Chem. 2003, 68, 5773-5776).

### SUMMARY OF THE INVENTION

Disclosed is a process for preparing a compound of formula A-N═C(D)(B), from a compound of formula A-N═C(E)(B) and a compound of formula D-M using an iron catalyst, where the process is represented by Equation (I)

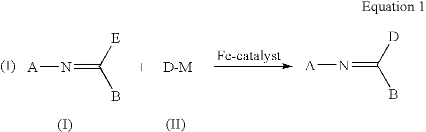

### BRIEF DESCRIPTION OF THE DRAWINGS

FIGS. 1a-c: Crude ¹H-NMR spectra of 44.

### DETAILED DESCRIPTION OF THE PREFERRED EMBODIMENT

General, high yielding and rapid synthetic transformations are of great demand in drug discovery. In this context, iron catalyzed reactions are of interest, as iron-catalysts (Blom, C.; Legros, J.; Paih, J. L. and Zani, L.; Chem. Rev. 2004, 104, 6217-6254). In addition iron catalyst are inexpensive, easy to handle and have a benign character. Fürstner, A. and Martin, R.; Chem. Lett. 2005, 34, 624-629. With the prospect of taking advantage of the wealth of amide bonds as synthons for carbon-carbon bond formations, metal catalyzed addition to an intermediate imidoyl chloride is of particular interest (eq. 1). Furthermore, applying such transformations on privileged structures gives the potential to discover novel pharmacological activities, comparable with the discovery of the change in biological mechanism when clozapine is transformed into its major metabolite N-desmethylclozapine. Horton, D. A.; Bourne, G. T. and Smythe, M. L.; Chem. Rev. 2003, 103, 893-930; Abrous, L.; Hynes, J.; Fredrich, S. R.; Smith, A. B. and Hirschmann, R.; Org Lett. 2001, 3, 1089-1092; Weiner, D. M. et al.; Psycopharm. 2004. The few metal catalyzed cross-coupling reactions of imidoyl chlorides reported have used the expensive Pd or toxic Ni catalysts. Kobayashi, T.; Sakakura, T. and Tanaka, M.; Tetrahedron Lett. 1985, 26, 3463-3466; Davis, F. A.; Mohanty, P. K.; Burns, D. M. and Andemichael, Y. W.; Org. Lett. 2000, 2, 3901-3903. Nadin et al. published the so far only known example of an imidoyl chloride cross-coupling reaction generating an sp²-sp³ bond, using a Pd catalysed Negishi reaction. Nadin, A. et al.; J. Org. Chem. 2003, 68, 2844-2852. Reported herein are the first iron-catalyzed cross-coupling reactions of imidoyl chlorides with Grignard reagents.

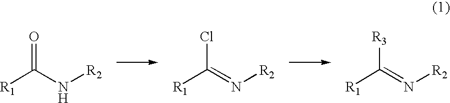

A procedure has been developed, and disclosed herein, for the synthesis of imines or related compounds from imidoyl halides/sulfonates/triflates and phosphates using iron catalyzed cross coupling with organometallic reagents. This new procedure takes advantage of amide bonds as synthons for carbon-carbon bond formations and provides a tool for generating novel compounds. This new procedure has advantages compared over established methodology for synthesis of this type of compounds. Most notable aspects are the following: (1) Iron catalyzed cross coupling reactions are fast and often high yielding; (2) Compared to other transition metals commonly used in cross coupling reactions, iron salts, complexes or precatalysts are toxicologically benign, cheap and stable; (3) Many iron salts and complexes are commercially available; and (4) There is no need for additional supporting ligands. Blom, C.; Legros, J.; Paih, J. L. and Zani, L. Chem. Rev. 2004, 104, 6217-6254.

The active iron catalyst is formed in situ under reaction conditions from suitable iron precatalysts. All iron compounds of the oxidation states −2, −1, 0, +1, +2, +3 can be used as such precatalysts, including metallic iron or intermetallic iron compounds if used in suitably dispersed form. This includes, but is not restricted to, FeF₂, FeF₂ 4H₂O, FeF₃ H₂O, FeCl₂, FeCl₂ 4H₂O, FeCl₃, FeCl₃ 6H₂O, FeCl₃(PPh₃), Fe(OEt)₂, Fe(OEt)₃, FeCl₂(PPh₃)₂, FeCl₂(dppe) [dppe=1,2-bis-(diphenylphosphino)ethane], Fe(acac)₂ [acac=acetylacetonate], Fe(acac)₃, tris-(trifluoroacetylacetonato)iron (III), tris-(hexafluoroacetylacetonato)iron (III), tris-(dibenzoylmethido)iron (III), tris-(2,2,6,6-tetramethyl-3,5-diheptanedionate)iron (III), FeBr₂, FeBr₃, FeI₂, Fe(II)acetate, Fe(II)oxalate, Fe(II)stearate, Fe(III)citrate hydrate, Fe(III)pivalate, Fe(II)-D-gluconate 2 H₂O, Fe(OSO₂C₆H₄Me)₃, Fe(OSO₂C₆H₄Me)₃ hydrate, FePO₄, Fe(NO₃)₃, Fe(NO₃)₃ 9 H₂O, Fe(ClO₄)₃ hydrate, FeSO₄, FeSO₄ hydrate, Fe₂(SO₄)₃, Fe₂(SO₄)₃ hydrate, K₃Fe(CN)₆, ferrocene, bis(pentamethylcyclopentadienyl)iron, bis(indenyl)iron, Fe(II)phtalocyanin, Fe(III)phtalocyanin chloride, Fe(CO)₅, Fe(salen)X [salen=N,N-ethylenebis(salicylidenamidato), X=Cl, Br, I], 5,10,15,20-tetraphenyl-21H,23H-porphin-iron(III) halide, 5,10,15,20-tetrakis(pentafluorophenyl)-21H,23H-porphin-iron(III) halide, activated Fe. (A. V. Kavaliunas et al.; Organometallics 1983, 2, 377-383; A. Fürstner; Angew. Chem. Int. Ed. Eng. 1993, 32, 164-189), iron-magnesium intermetallic compounds (L. E. Aleandri et al.; Chem. Mat. 1995, 7, 1153-1170; B. Bogdanovic et al.; Angew. Chem. Int. Ed. 2000, 39, 4610-4612). The precatalysts can be used in anhydrous or hydrated form. Preferred catalysts are those that are soluble or partly soluble in the reaction medium. The catalyst loading can be varied in a vide range, preferably between 0.01% and 20 mol % with regard to the substrates used.

Thus, in the first aspect, the present invention relates to a process for preparing a compound of formula A-N═C(D)(B), from a compound of formula A-N═C(E)(B) and a compound of formula D-M using an iron catalyst, where the process has is represented by Equation (I) 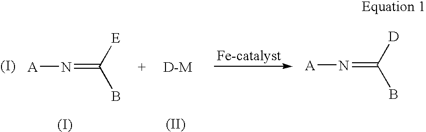
wherein
A and B are independently selected from the group consisting of optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁, or A and B taken together, along with the nitrogen atom to which A is attached and the carbon atom to which B is attached, form a ring;
E is selected from the group consisting of halide, sulfonate (—OSO₃R₂), and phosphonate (—OP(O)(OR₂ₐ)(OR_{2b}));
D is selected from group consisting of optionally substituted alkyl, optionally substituted alkenyl, optionally substituted alkynyl, optionally substituted cycloalkyl, optionally substituted cycloalkenyl, optionally substituted aryl, optionally substituted heteroaryl, and optionally substituted heteroalicyclyl;
M is selected from the group consisting of MgY, CaY, ZnY, MnY, and Mg derived metal reagents formed from reaction of MgY and other metal salts, such as Cu(CN)MgCl and Mn(Cl₂)MgCl;
Y is an anionic ligand
R₁, R₁ₐ and R_{1b} are independently selected from the group consisting of hydrogen, optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl;
R₂, R₂ₐ and R_{2b} are independently selected from the group consisting of haloalkyl, optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl; and
Z is O (oxygen) or S (sulfur).

In some embodiments, compounds (I) and (II) are isolated, presynthesized chemical entities that are brought together for the reaction of Equation (I) to take place. In other embodiments, or either of (I) or (II) or both can be generated in-situ from suitable precursors that are brought together for the reaction of Equation (I) to take place. The present disclosure contemplates all the possible permutations of presynthesized and in-situ generated (I) and (II). The N═C double bond depicted in Compound (I) generating geometrical isomers can be defined as either E or Z.

In some embodiments, where A and B taken together, along with the nitrogen and carbon atoms to which A and B are respectively attached form a ring, the ring is optionally fused with another ring system, such as an optionally substituted aryl, an optionally substituted heteroaryl, and an optionally substituted heteroalicyclyl

In some embodiments, the present invention relates to a process for preparing a compound of Formula IV as shown in Equation 2 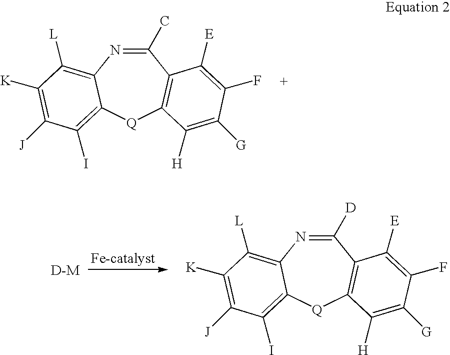
wherein
C is selected from the group consisting of halide, sulfonate (—OSO₃R₂), and phosphonate (—OP(O)(OR₂ₐ)(OR_{2b}));
D is selected from group consisting of alkyl, alkenyl, alkynyl, cycloalkyl, cycloalkenyl, optionally substituted aryl, optionally substituted heteroaryl, and optionally substituted heteroalicyclyl;
M is MgY;
Y is an anionic ligand;
Q is selected from the group consisting of NR₁, N⁺—O⁻, O, S, S═O, O═S═O, CR₁R₂, C═O, and SiR₁R₂;
E, F, G, H, I, J and L are each independently selected from the group consisting of hydrogen, alkyl, alkenyl, alkynyl, cycloalkyl, cycloalkenyl, optionally substituted aryl, optionally substituted heteroaryl, optionally substituted heteroalicyclyl, halogen, nitro, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;
K is selected from the group consisting of alkyl, alkenyl, alkynyl, cycloalkyl, cycloalkenyl, optionally substituted aryl, optionally substituted heteroaryl, optionally substituted heteroalicyclyl, halogen, hydroxyl, nitro, sulfenyl, sulfinyl, sulfonyl, haloalkyl, haloalkoxy, —CN, —C(=Z)R₁, —C(=Z)OR₁, —C(=Z)NR₁ₐR_{1b}, C(=Z)N(R₁)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;
R₁, R₁ₐ and R_{1b} are independently selected from the group consisting of hydrogen, optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl;
R₂, R₂ₐ and R_{2b} are independently selected from the group consisting of: haloalkyl, optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl; and
Z is O (oxygen) or S (sulfur).

### Definitions

Whenever a group of this invention is described as being “optionally substituted” that group may be unsubstituted or substituted with one or more of the indicated substituents. Likewise, when a group is described as being “unsubstituted or substituted” if substituted, the substituent may be selected from the same group of substituents.

Unless otherwise indicated, when a substituent is deemed to be “optionally subsituted,” it is meant that the subsitutent is a group that may be substituted with one or more group(s) individually and independently selected from cycloalkyl, aryl, heteroaryl, heterocyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

As used herein, “Cₘ to Cₙ” in which “m” and “n” are integers refers to the number of carbon atoms in an alkyl, alkenyl or alkynyl group or the number of carbon atoms in the ring of a cycloalkyl or cycloalkenyl group. That is, the alkyl, alkenyl, alkynyl, ring of the cycloalkyl or ring of the cycloalkenyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)— and (CH₃)₃CH—. If no “m” and “n” are designated with regard to an alkyl, alkenyl, alkynyl, cycloalkyl or cycloalkenyl group, the broadest range described in these definitions is to be assumed.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds of the invention may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl.

The alkyl group may be substituted or unsubstituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, —NR₁ₐR_{1b}, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substitutent may be substituted with one of the above substituents.

“Lower alkylene groups” are straight-chained tethering groups, forming bonds to connect molecular fragments via their terminal carbon atoms. Examples include but are not limited to methylene (—CH₂—), ethylene (—CH₂CH₂—), propylene (—CH₂CH₂CH₂—) or butylene (—(CH₂)₄—) groups.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene. An aryl group of this invention may be substituted or unsubstituted. When substituted, hydrogen atoms are replaced by substituent group(s) that is(are) one or more group(s) independently selected from alkyl, cycloalkyl, aryl, heteroaryl, heteroalicyclyl, hydroxy, protected hydroxyl, alkoxy, aryloxy, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, —NR₁ₐR_{1b} and protected amino.

As used herein, “heteroaryl” refers to a monocyclic or multicyclic aromatic ring system (a ring system with fully delocalized pi-electron system), one or two or more fused rings that contain(s) one or more heteroatoms, that is, an element other than carbon, including but not limited to, nitrogen, oxygen and sulfur. The heteroaryl group may be optionally fused to a benzene ring. Examples of heteroaryl rings include, but are not limited to, furan, thiophene, phthalazinone, pyrrole, oxazole, thiazole, imidazole, pyrazole, isoxazole, isothiazole, triazole, thiadiazole, pyran, pyridine, pyridazine, pyrimidine, pyrazine and triazine. A heteroaryl group of this invention may be substituted or unsubstituted. When substituted, hydrogen atoms are replaced by substituent group(s) that is(are) one or more group(s) independently selected from alkyl, cycloalkyl, aryl, heteroaryl, heteroalicyclyl, hydroxy, protected hydroxyl, alkoxy, aryloxy, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, —NR₁ₐR_{1b} and protected amino

As used herein, “alkoxy” refers to the formula —OR wherein R is an alkyl is defined as above, e.g. methoxy, ethoxy, n-propoxy, 1-methylethoxy (isopropoxy), n-butoxy, iso-butoxy, sec-butoxy, tert-butoxy, amoxy, tert-amoxy and the like.

As used herein, “alkylthio” refers to the formula —SR wherein R is an alkyl is defined as above, e.g. methylmercapto, ethylmercapto, n-propylmercapto, 1-methylethylmercapto (isopropylmercapto), n-butylmercapto, iso-butylmercapto, sec-butylmercapto, tert-butylmercapto, and the like.

An alkyl group of this invention may be substituted or unsubstituted. When substituted, hydrogen atoms are replaced by substituent group(s) that is(are) one or more group(s) independently selected from cycloalkyl, aryl, heteroaryl, heteroalicyclyl, hydroxy, protected hydroxyl, alkoxy, aryloxy, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, —NR₁ₐR_{1b} and protected amino.

“Aralkyl groups” are aryl groups connected, as substituents, via a lower alkylene group. The aryls groups of aralkyl may be substituted or unsubstituted Exampels includes but are not limited to benzyl, substituted benzyl, 2-phenylethyl, 3-phenylpropyl, naphtylalkyl.

“Heteroaralkyl groups” are understood as heteroaryl groups connected, as substituents, via a lower alkylene group. The heteroaryls groups of heteroaralkyl may be substituted or unsubstituted. Exampels includes but are not limited to 2-thienylmethyl, 3-thienylmethyl, furylmethyl, thienylethyl, pyrrolylalkyl, pyridylalkyl, isoxazollylalkyl, imidazolylalkyl, and their substituted as well as benzo-fused analogues.

As used herein, “aryloxy” and “arylthio” refers to RO— and RS—, in which R is an aryl, such as but not limited to phenyl.

As used herein, “alkenyl” refers to an alkyl group that contains in the straight or branched hydrocarbon chain one or more double bonds. An alkenyl group of this invention may be unsubstituted or substituted. When substituted, the substituent(s) may be selected from the same groups disclosed above with regard to alkyl group substitution.

As used herein, “alkylidene” refers to a divalent group, such as ═CR′R″, which is attached to one carbon of another group, forming a double bond, Alkylidene groups include, but are not limited to, methylidene (═CH₂) and ethylidene (═CHCH₃). As used herein, “arylalkylidene” refers to a group to an alkylidene group in which either R′ and R″ is an aryl group.

As used herein, “alkynyl” refers to an alkyl group that contains in the straight or branched hydrocarbon chain one or more triple bonds. An alkynyl group of this invention may be unsubstituted or substituted. When substituted, the substituent(s) may be selected from the same groups disclosed above with regard to alkyl group substitution.

As used herein, “acyl” refers to an “RC(═O)—” group with R as defined above.

As used herein, “cycloalkyl” refers to a completely saturated (no double bonds) mono- or multi-cyclic hydrocarbon ring system. Cycloalkyl groups of this invention may range from C₃ to C₁₀, in other embodiments it may range from C₃ to C₆. A cycloalkyl group may be unsubstituted or substituted. If substituted, the substituent(s) may be selected from those indicated above with regard to substitution of an alkyl group.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). A cycloalkenyl group of this invention may be unsubstituted or substituted. When substituted, the substituent(s) may be selected from the groups disclosed above with regard to alkyl group substitution.

As used herein, “heteroalicyclic” or “heteroalicyclyl” refers to a stable 3- to 18 membered ring which consists of carbon atoms and from one to five heteroatoms selected from the group consisting of nitrogen, oxygen and sulfur. For the purpose of this invention, the “heteroalicyclic” or “heteroalicyclyl” may be monocyclic, bicyclic, tricyclic, or tetracyclic ring system, which may include fused or bridged ring systems; and the nitrogen, carbon and sulfur atoms in the “heteroalicyclic” or “heteroalicyclyl” may be optionally oxidized; the nitrogen may be optionally quatemized; and the rings may also contain one or more double bonds provided that they do not form a fully delocalized pi-electron system in the rings. Heteroalicyclyl groups of this invention may be unsubstituted or substituted. When substituted, the substituent(s) may be one or more groups independently selected from the group consisting of halogen, hydroxy, protected hydroxy, cyano, nitro, alkyl, alkoxy, acyl, acyloxy, carboxy, protected carboxy, amino, protected amino, carboxamide, protected carboxamide, alkylsulfonamido and trifluoromethanesulfonamido. Examples of such “heteroalicyclic” or “heteroalicyclyl” include but are not limited to, azepinyl, acridinyl, carbazolyl, cinnolinyl, dioxolanyl, imidazolinyl, morpholinyl, oxiranyl, piperidinyl N-Oxide, piperidinyl, piperazinyl, pyrrolidinyl, 4-piperidonyl, pyrazolidinyl, 2-oxopyrrolidinyl, thiamorpholinyl, thiamorpholinyl sulfoxide, and thiamorpholinyl sulfone.

The ring systems of of the cykloalkyl, heteroalicyclic (heteroalicyclyl) and cykloalkenyl groups may be composed of one ring or two or more rings which may be joined together in a fused, bridged or spiro-connected fashion.

As used herein, “halide”, “halo” or “halogen” refers to F (fluoro), Cl (chloro), Br (bromo) or I (iodo).

As used herein, “haloalkyl” refers to an alkyl group in which one or more of the hydrogen atoms are replaced by halogen. Such groups include but are not limited to, chloromethyl, fluoromethyl, difluoromethyl, trifluoromethyl and 1 -chloro-2-fluoromethyl, 2-fluoroisobutyl.

As used herein, “haloalkoxy” refers to RO-group in which R is a haloalkyl group. Such groups include but are not limited to, chloromethoxy, fluoromethoxy, difluoromethoxy, trifluoromethoxy and 1-chloro-2-fluoromethoxy, 2-fluoroisobutyoxy.

An “O-carboxy” group refers to a “RC(═O)O—” group with R as defined above.

A “C-carboxy” group refers to a “—C(═O)R” group with R as defined above.

An “acetyl” group refers to a CH₃C(═O)— group.

A “trihalomethanesulfonyl” group refers to an “X₃CSO₂—” group wherein X is a halogen.

A “cyano” group refers to a “—CN” group.

An “isocyanato” group refers to an “—NCO” group.

A “thiocyanato” group refers to a “—CNS” group.

An “isothiocyanato” group refers to an “—NCS” group.

A “sulfinyl” group refers to an “—S(═O)—R” group with R as defined above.

A “sulfonyl” group refers to an “SO₂R” group with R as defined above.

An “S-sulfonamido” group refers to a “—SO₂NR₁ₐR_{1b}” group with R₁ₐ and R_{1b} as defined above.

An “N-sulfonamido” group refers to a “RSO₂N(R₁ₐ)—” group with R and R₁ₐ as defined above.

A “trihalomethanesulfonamido” group refers to an “X₃CSO₂N(R)—” group with X as halogen and R as defined above.

An “O-carbamyl” group refers to a “—OC(═O)NR₁ₐR_{1b}” group with R₁ₐ and R_{1b} as defined above.

An “N-carbamyl” group refers to an “ROC(═O)NR₁ₐ—” group with R₁ₐ and R as defined above.

An “O-thiocarbamyl” group refers to a “—OC(═S)—NR₁ₐR_{1b}” group with R₁ₐ and R_{1b} as defined above.

An “N-thiocarbamyl” group refers to an “ROC(═S)NR₁ₐ—” group with R₁ₐ and R as defined above.

A “C-amido” group refers to a “—C(═O)NR₁ₐR_{1b}” group with R₁ₐ and R_{1b} as defined above.

An “N-amido” group refers to a “RC(═O)NR₁ₐ—” group with R and R₁ₐ as defined above.

As used herein, an “ester” refers to a “—C(═O)OR” group with R as defined above.

As used herein, an “amide” refers to a “—C(═O)NR₁ₐR_{1b}” group with R₁ₐ and R_{1b} as defined above.

Any unsubstituted or monosubstituted amine group on a compound herein can be converted to an amide, any hydroxyl group can be converted to an ester and any carboxyl group can be converted to either an amide or ester using techniques well-known to those skilled in the art (see, for example, Greene and Wuts, Protective Groups in Organic Synthesis, 3^{rd} Ed., John Wiley & Sons, New York, N.Y., 1999).

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature See, Biochem. 1972, 11, 942-944.

The following abbreviations are used throughout the present disclosure:
AcOH acetic acid
anhyd anhydrous
Aq. aqueous
Bu butyl
Cat. catalyst
Cbz. benzyloxycarbonyl
CDI 1,1′-carbonyldiimidazole
d doublet
δ chemical shift in ppm
DA dopamine
DCM dichlormethan
dd double doublet
DMAP 4-dimethylaminopyridine
DME demethoxyethane
DMF N,N-dimethylformamide
DMSO dimethyl sulfoxide
dt double triplet
EDCI 1-(3-dimethylaminopropyl)-3-ethyl-carbodiimide hydrochloride
e.g. example given
EPS extrapyramidal symptoms
Eq. equivalent(s)
Et ethyl
EtOAc ethyl acetate
EtOH ethanol
Et₂O diethyl ether
h hour(s)
HOBt 1-hydroxybenzothiazole
Hz herz
iPrMgCl isopropylmagnesium chloride
J coupling constant
m multiplet
M muscarinic receptor
MeMgCl methylmagnesium chloride
MeOH methanol
Min minute(s)
MW microwave
NDMC N-desmethylclozapine
NH₄OAc ammonium acetate
NMP N-methyl-pyrrolidone
NMR nuclear magnetic resonance
Pd palladium
Pd/C palladium on activated carbon
Ph phenyl
PPh₃ triphenyl phosphine
PhMgCl phenylmagnesium chloride
ppm parts per million
rt room temperature
s singlet
SAR structure-activity relationship analysis
t triplet
tBuMgCl tert-Butylmagnesium choride
TEA triethylamine
THF tetrahydrofuran
TLC thin layer chromatography
TFP tri furylphosphine

As used herein, the phrase “taken together form a ring” when referring to two “R” groups means that the “R” groups are joined together to form a cycloalkyl, aryl, heteroaryl or heteroalicyclyl group, along with the atoms to which the “R” groups are attached. Thus, the atoms to which the “R” groups are attached form a part of the rign. For example, without limitation, if R₁ₐ and R_{1b} of an NR₁ₐR_{1b} group are indicated to be “taken together to form a ring,” it means that they are covalently bonded to one another at their terminal atoms to form a ring:

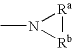

It is understood that, in any compound of this invention having one or more chiral centers, if an absolute stereochemistry is not expressly indicated, then each center may independently be of R-configuration or S-configuration or a mixture thereof. Thus, the compounds provided herein may be enatiomerically pure or be stereoisomeric mixtures. In addition it is understood that, in any compound of this invention having one or more double bond(s) generating geometrical isomers that can be defined as E or Z each double bond may independently be E or Z a mixture thereof. Likewise, all tautomeric forms are also intended to be included.

Synthesis

Synthesis of Tricyclic Imidoyl Chlorides

A total of four lactams were prepared as starting material for the imidoyl chlorides by two procedures: A and B. Table 1 presents the obtained lactams including 15 and 18, which were commercially available at Chempacific and Aldrich, respectively.

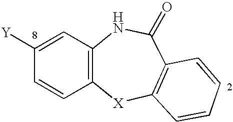

TABLE 1
Synthesis of tricyclic lactams.

| Lactam | X | Y | Procedure |
|---|---|---|---|
| 15 | NH | Cl | — |
| 16 | NH | H | A |
| 17 | O | Cl | B |
| 18 | O | H | — |
| 19 | S | Cl | B |
| 20 | S | H | B |

In procedure A (Scheme 1), 2-aminobenzoic acid was reacted with an excess of 2-fluoronitrobenzene and cesium carbonate in DMF at 140° C. An excess of the 2-fluoronitrobenzene was used to ensure complete consumption of the aminobenzoic acid in order to simplify workup by acid/base extraction. Extractive workup and recrystallization from methanol gave 21 in a good yield.

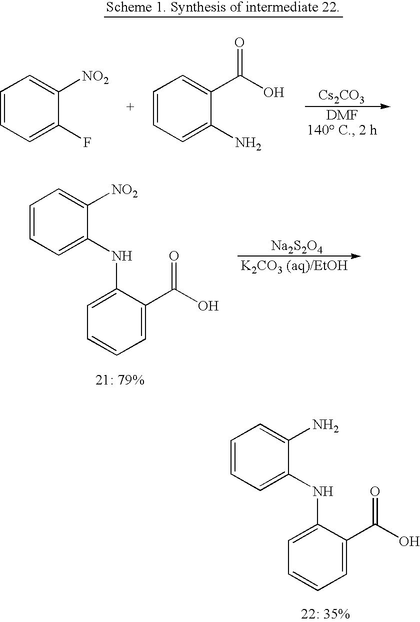

The reduction of the nitro-group was carried out by dissolving 21 in an ethanol/alkaline aqueous solution. Adding sodium dithionite gave, within minutes, complete reduction. The crude product was sufficiently pure to be employed in the next step without further purification; however, the yield was low (35%). The special character of 22 increases the aqueous solubility and this made it difficult to obtain complete extraction back into the organic phase. Lactam 16 was then synthesized at room temperature using EDCI as coupling agent, which gave a good yield (91%, Scheme 2).

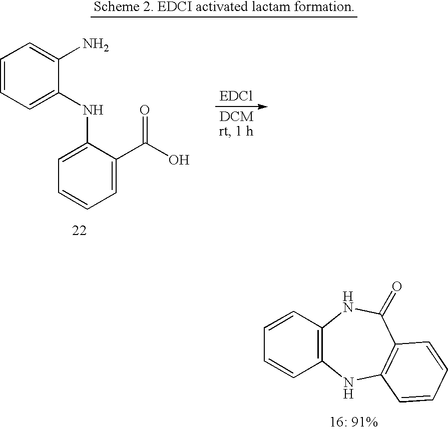

In procedure B, the appropriate benzoic methyl esters were reacted in DMF with an excess of 2-fluronitrobenzene derivatives (Scheme 3). The nucleophilic aromatic substitution (NAS) was performed according to procedure A, however the reaction temperature could be lowered (40-60° C.). A higher acidic character of phenol and thiol compared to aniline could in this case explain this difference in reactivity.

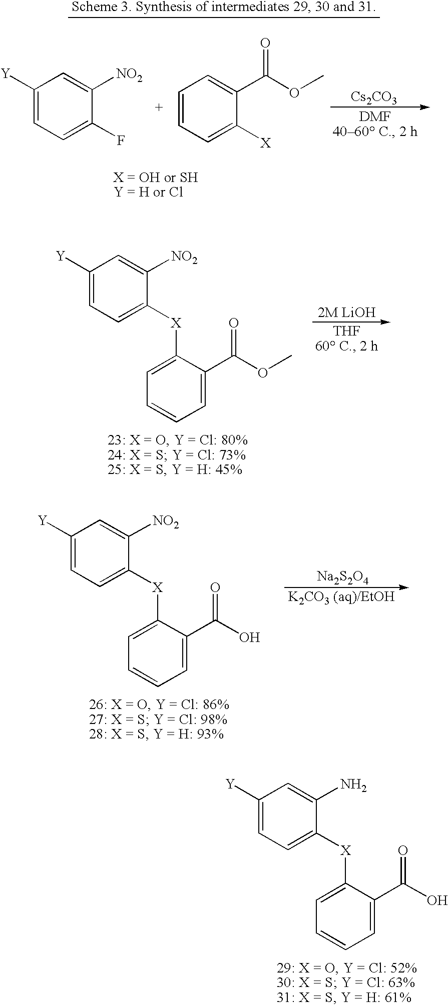

As exemplified in Scheme 3, the aromatic substitution gave moderate to good yields (45 to 80%). In order to prevent undesired disulfide formation when methyl thiosalicylate was applied, the solvent was degassed with nitrogen. The methyl esters were then hydrolyzed in THF and 2 M LiOH (aq.), resulting in quantitative yields of 26 to 28. Reduction of the nitro-groups was preformed using the same conditions as in procedure A, which gave moderate yields (52 to 63%), of the desired products 29 to 31. Synthesis of the seven-member ring was first attempted using ECDI as coupling reagents under conditions described in procedure A, however, no formation of the desired lactams was detected. Analysis by LC-MS of the reaction mixture showed that the activated intermediate 32 was the major product (Scheme 4).

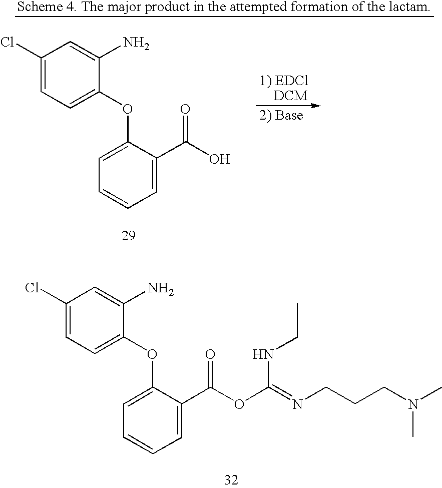

The activated intermediate 32 showed unexpectedly high stability and even though the reaction was heated in CHCl₃ for several hours no lactam was formed. The same problem was seen when 30 and 31 were applied directly in the ring closing reaction.

TABLE 2
Calculation of the angle between the two-aryl rings.

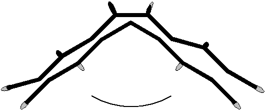

| X | Angle |
|---|---|
| NH | 142° |
| O | 135° |
| S | 121° |

Geometry optimized using B3LYP/6-31G*, Spartan '04 by Wavefunction Inc, http://www.wavefun.com

Replacement of the nitrogen atom to the oxygen or sulfur atom clearly decreases the angle between the two aryl-rings in the lactam as seen in table 2. This might indicate a greater difficulty in forming the lactam for 17, 19 and 20 compared to 16. In order to solve this problem, compound 29 to 31 were reacted in microwave together with EDCI, HOBt, DMAP and TEA (table 4).

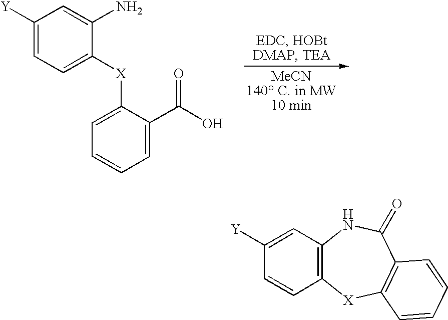

TABLE 3
Synthesis of lactams

| Lactam | Yield^{a} (%) |
|---|---|
| 17 | 66 |
| 19 | 63 |
| 20 | 51 |

^{a}Isolated yields.

This afforded the desired lactams in moderate yields. Further studies were not conducted to determine if the influence of the additives or the higher reaction temperature in the microwave were responsible for the successful ring closure,

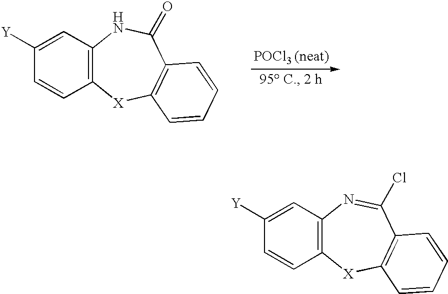

TABLE 4
Synthesis of imidoyl chlorides.

| Imidoyl chloride | X | Y | Yield^{a} (%) |
|---|---|---|---|
| 33 | S | H | 65 |
| 34 | S | Cl | 74 |
| 35 | O | H | 78 |
| 36 | O | Cl | 84 |

^{a}Isolated yields.

Lactams 17 to 20 were then reacted with refluxing POCl₃ for two hours, which, after extractive workup and column chromatography, gave the imidoyl chloride 33 to 36 in good yields (table 4). However, in case of imidoyl chloride 1, neat POCl₃ was probably too harsh, thus, the crude product contained several unidentified by-products. An alternative milder procedure was employed using equal amounts of POCl₃ and N,N-dimethyl aniline in refluxing toluene (Scheme 5). Wade, P. C.; Vogt, B. R.; Toeplitz, B.; Puar, M. S. and Gougoutas, J. Z.; J. Org. Chem. 1979, 44, 88.

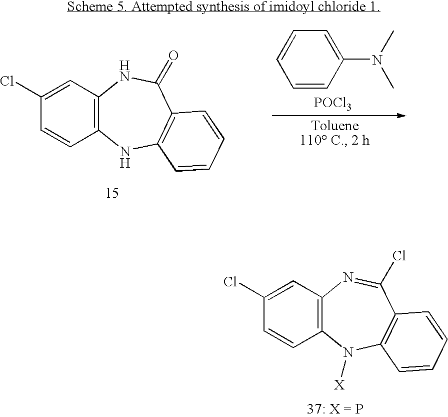

Although the expected formation of the imidoyl chloride moiety was successful, analysis by ¹H-NMR and TLC indicated that the major product in the crude mixture was some type of by-product 37 presumably formed from the reaction of phosphorous oxychloride with 5-N atom. Applying the crude product 37 in a cross-coupling reaction utilizing Suzuki conditions did not result in the expected cross-coupling product. Instead, the starting material 1 returned without the 5-N-phosphor substituent. This result inspired us to heat 37 in a two-phase system with aqueous sodium carbonate (Scheme 6) in order to obtain 1 from 37.

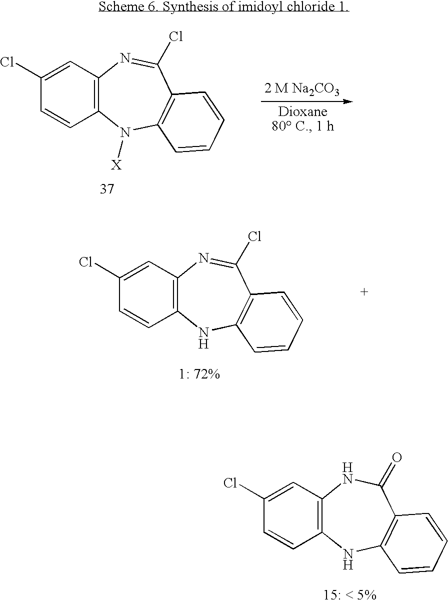

As exemplified in Scheme 6 hydrolysis of the 5-N-P moiety using the previously described conditions was successful and 1 was isolated in a good yield with only minor amounts of the hydrolysed compound 15. As mentioned, imidoyl chlorides are in general described to be highly reactive compounds, hence, they ought to have similar reactivity pattern as acyl chlorides. Therefore, these harsh conditions did not completely hydrolyze the imidoyl chloride that possesses a higher stability than expected. In addition, all the obtained imidoyl chlorides 1, 33, 34, 35 to 36 were stable towards aqueous workup and column chromatography, and could in most cases be stored in a freezer for months with only minor hydrolysis to the lactam, with the exception of 35, which quite rapidly hydrolyzed even in storage at subzero temperature. The stability of the imidoyl chlorides could be explained by conjugation of the two aromatic systems. This indicates a greater resemblance of this class of imidoyl chlorides with halo substituted heteroaromatic compounds such as 2-chloropyridine 38 than with acyl halides 39 (chart 1). 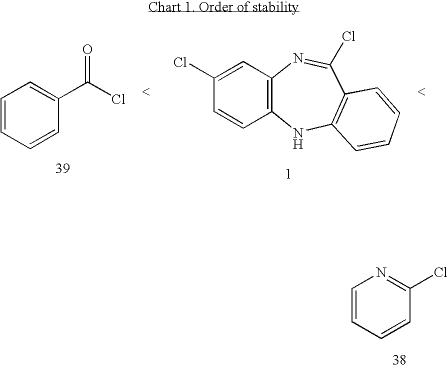
Palladium-catalyzed Cross-coupling Reactions

The cross-coupling reaction of imidoyl chlorides with alkylzincs (Negishi) and boron boronic acid reagents (Suzuki), respectively, was investigated in order to find optimum conditions for the desired cross-coupling. In the initial studies, imidoyl chloride 1 and 35 was applied since the corresponding lactam was commercially available (Scheme 7).

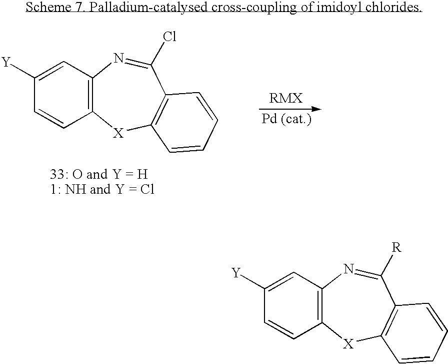

The experiments were conducted utilizing thermal or microwave assisted heating using 0.20 mmol imidoyl chloride, 0.40 mmol organometallic halide and 5-10 mol % catalyst.

TABLE 5
Screening of palladium-catalysts.

| Entry | Substrate | RMX | Conditions^{a} | Catalyst | Product | Yield^{b} (%) |
|---|---|---|---|---|---|---|
| 1 | 35 | BuZnBr | A | Pd(PPh₃)₄ | 40 | 30 |
| 2 | 35 | BuZnBr | A | Pd(P(t-Bu)₃)₂ | 40 | (44) |
| 3 | 35 | BuZnBr | A | PdCl₂(PPh₃)₂ | 40 | (50) |
| 4 | 1 | CN(CH₂)₃ZnBr | B | Pd₂(dba)₃/TFP | 41 | 42 (85) |
| 5 | 1 | C₆H₁₁ZnBr | B | Pd₂(dba)₃/TFP | 42 | 65 (81) |
| 6 | 35 | BuB(OH)₂ | C | Pd(PPh₃)₄ | — | 0 |

^{a}Conditions: (A): THF/NMP, rt, 0.5-2 h; (B): THF/NMP, MW 140° C., 5 min; (C): DME/EtOH, K₂CO₃, 75° C., 17 h.
^{b}Isolated yields. ¹H-NMR yields using anisole as an internal standard is in parentheses.

Initial experiments with the cross-coupling of 35 with butylzinc bromide using Pd catalysts provided low yields (entry 1-3, table 5 showed complete conversion of the starting material after 0.5 to 2 hours at room temperature; however, TLC analysis indicated several by-products presumably arising from decomposition of the starting material. The yields were moderate, giving 50% yield of 40 at the best, when PdCl₂(PPh₃)₂ was employed as catalyst (entry 2). Entry 4 and 5 shows cross-coupling reactions using microwave assisted heating of 1 with 3-cyanopropylzinc bromide (entry 4) and cyclohexylzinc bromide (entry 5), respectively. Using the catalyst/ligand system Pd₂(dba)₃/Tri-2-furylphosphine (TFP) afforded a yield of 85% of 41 according to NMR analysis using internal standard, however, the isolated yield was only 42% (entry 4). This large difference in outcome is probably due to difficulties separating the product from dibenzylideneacetone (dba) by column chromotography despite testing several different solvent combinations. The same isolation problem arose in the cross-coupling of 1 with cyclohexylzinc bromide using Pd₂(dba)₃/TPF as catalyst system (entry 5). However, a minor difference between the yield determined with internal standard compared to isolated yield of 42 (81% and 65%, respectively) was seen in this case. Substrate 35 was also applied in a cross-coupling reaction with butylboronic acid under Suzuki conditions (entry 6). After a reaction time of 17 hours at 75° C. TLC analysis indicated complete conversion of the starting material. However, ¹H-NMR analysis of the crude product showed that the desired product was not formed, presumably due to decomposition of the imidoyl chloride.

Screening of Metal Salts

A wide range of new cross-coupling reactions of organic halides has been developed by the combination of Grignard or zinc reagents, respectively, with simple transition metal salts such as iron, copper, manganese and cobalt. Fürstner, A. and Martin, R.; Chemistry Lett. 2005, 34, 624-629; Knochel, P.; Yeh, M. C. P.; Berk, S. C. and Talbert, J.; J. Org. Chem. 1988, 53, 2390-2392; Knochel, P. and Dübner, F.; Angew. Chem. 1999, 38, 379-381; Dohle, W.; Lindsay, D. M. and Knochel, P.; Org. Lett. 2001, 3, 2871-2873; Malosh, C. F. and Ready, J. M.; J. Am. Chem. Soc. 2004, 126, 10240-10241; Cahiez, G. and Laboue, B.; Tetrahedron Lett. 1992, 33, 4439-44; Cahiez, G.; Luart, D. and Lecomte, F.; Org. Letters 2004, 6, 4395-4398; Reddy, K. and Knochel, P.; Angew. Chem. 1996, 35, 1700-1701. Different metal salts were screened for the cross-coupling of imidoyl chloride 35 with butylmagnesium chloride. The influence of varying the metal salt and the reaction conditions in the cross-coupling of 35 with butylmagnesium chloride was investigated (Scheme 8).

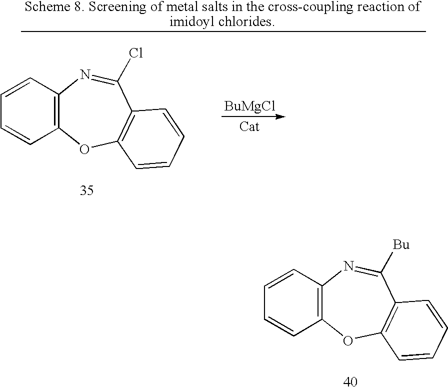

All reactions were conducted at room temperature using 0.20 mmol imidoyl chloride, 0.40 mmol butylmagnesium chloride and 5-10 mol % of the metal salt.

TABLE 6
Screening of metal salts

| Entry | Catalyst | Conditions^{a} | Yields^{b} (%) |
|---|---|---|---|
| 1 | None | A | 42 |
| 2 | None | A | 20 |
| | | (+NMP) | |
| 3 | Fe(acac)₃ | B | 22 |
| 4 | CoBr₂ | B | 17 |
| 5 | Co(acac)₃ | B | 22 |
| 6 | CoBr₂ | B | 56 |
| | | (+NMP) | |
| 7 | Co(acac)₃ | B | 50 |
| | | (+NMP) | |
| 8 | CuCl₂ | C | 52 |
| | | (+NMP) | |
| 9 | CuCN | C | 64 |
| | | (+NMP) | |
| 10 | MnCl₂ | B | 94 |
| | | (+NMP) | |
| 11 | Fe(acac)₃ | B | 96 |
| | | (+NMP) | |
| 12 | FeCl₃ | B | 95 |
| | | (+NMP) | |

^{a}Conditions: (A): THF, rt, 30 min; (B): THF, rt, 5 min; (C): THF, rt, 24 h.
^{b}¹H-NMR yields based on toluene as an internal standard.

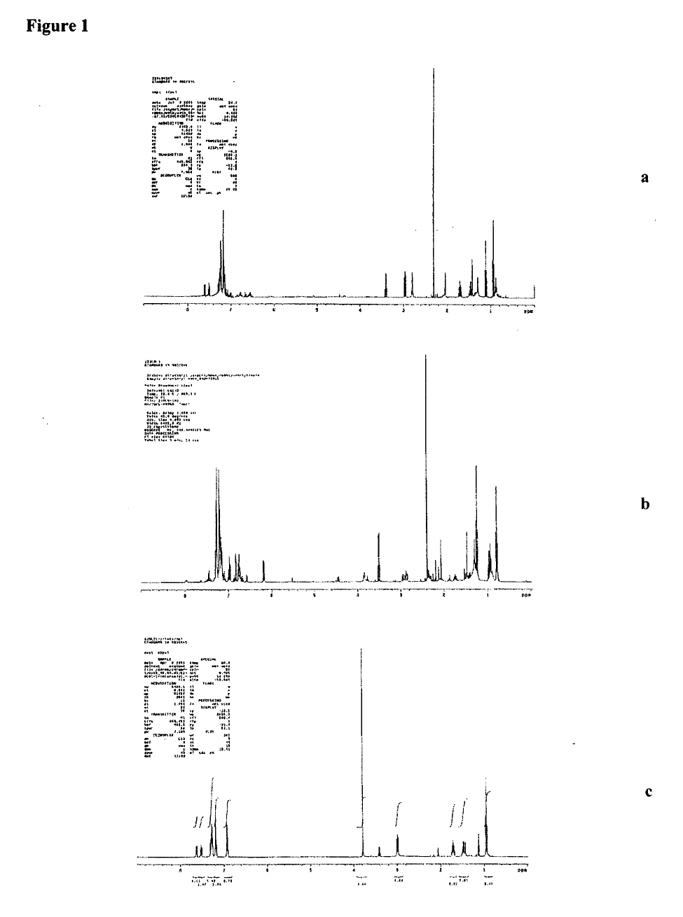

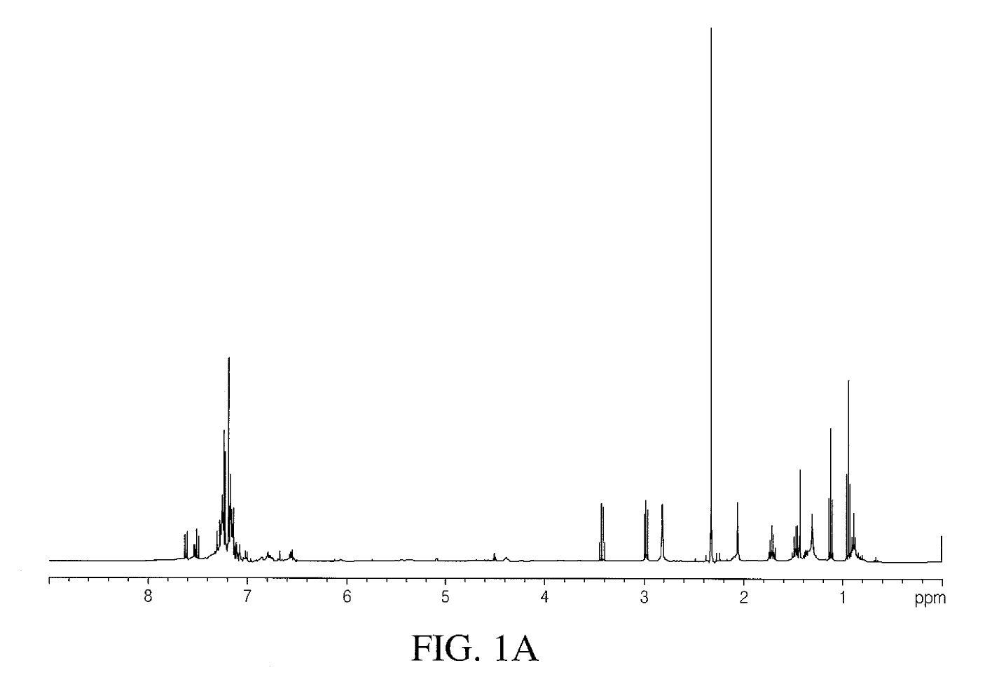

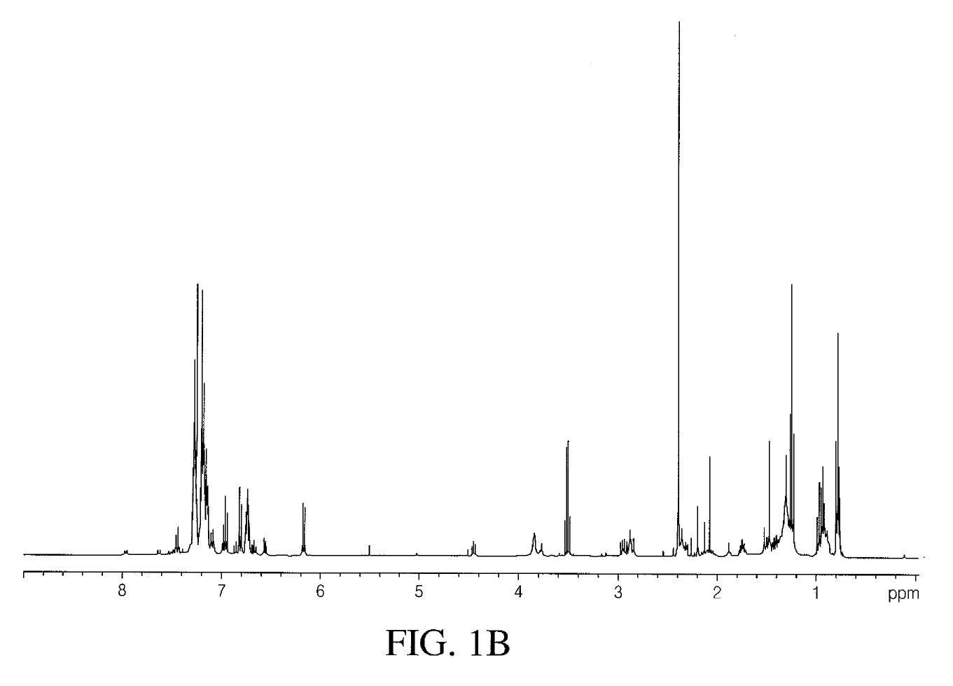

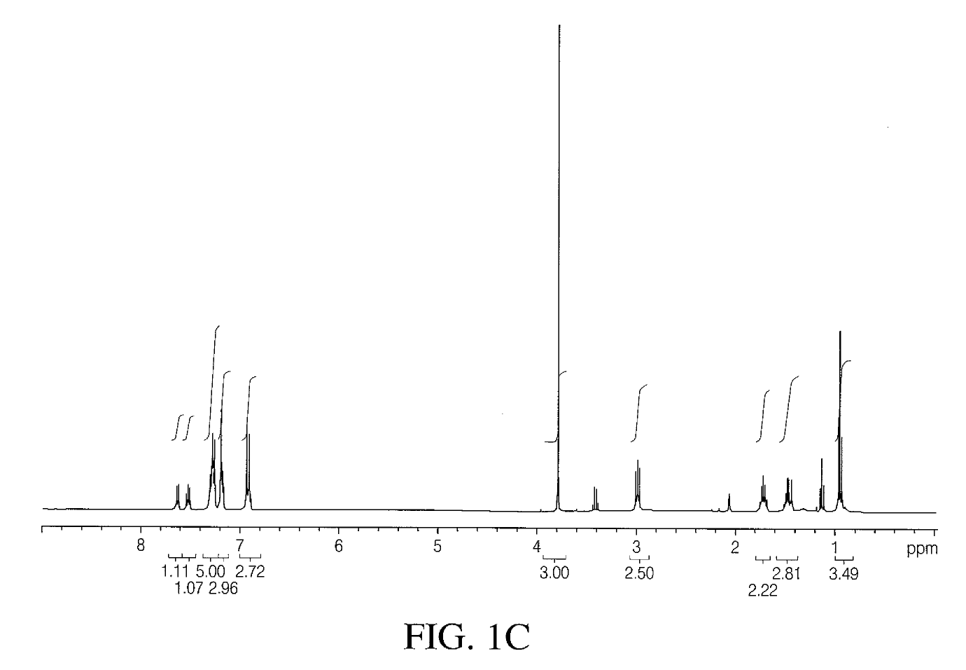

First, an uncatalysed test-reaction was carried out giving a low yield, 42%, at ambient temperature in THF with a 30 min reaction time (entry 1, table 6). However, addition of 5 mol % of Fe(acac)₃ at room temperature gave a rapid reaction with complete conversion of the imidoyl chloride within 5 min although the yield (22%) was low compared to the uncatalysed reaction (entry 3). The same low yields (17-22%) were obtained by applying CoBr₂ and Co(acac)₃, respectively (entry 4 and 5). Cahiez et al. first demonstrated the crucial effect of NMP in this type of reaction. Cahiez, G. and Avedissian, H.; Synthesis 1998, 1199-1205. Repeating the experiments in the presence of NMP significantly improved the yields. Thus, 35 reacted with butylmagnesium chloride, in the presence of 5 mol % CoBr₂, to give 17% (entry 4) of the coupling product whereas by adding NMP as co-solvent the yield increased to 56% (entry 6). The same pattern was observed for Co(acac)₂ (entry 7) resulting in an improved yield (50%). Employing the copper salts CuCl₂ and CuCN (entry 8 and 9) in the presence of NMP gave 52 and 64% yield, respectively. However, low solubility was observed of the copper salts, which could explain the slow conversion (24 h) of the imidoyl chloride. Stoichiometric amounts of manganese salts have been applied in cross-coupling reactions of various substrates e.g. imidoyl chlorides with Grignard reagents. Bouisset, M; Bousquet, A. and Heymes, A.; DE 3831533 1988. A test reaction was performed using a catalytic amount of MnCl₂ (entry 10). Addition of 10 mol % of MnCl₂ provided a rapid and high yielding cross-coupling reaction of 35 with butylmagnesium chloride (5 min, 94%). The good yield of product in this case was unexpected since the solubility of the metal salt in THF/NMP was low. Repeating the experiment with Fe(acac)₃ (entry 3) in the presence of NMP as co-solvent was also successful (entry 11). Addition of butylmagnesium chloride to a solution of 35 and Fe(acac)₃ caused, as reported in the iron-catalyzed alkyl-aryl cross-coupling reaction by Fürstner and co-workers, an immediate color change from red to dark brown with an increase in temperature (25 to 42° C.). Fürstner, A.; Leitner, A.; Méndez, M. and Krause, H.; J. Am. Chem. Soc. 2002, 13856-13863. Analysis (TLC) after 5 min indicated full conversion of the starting material and one single spot. An extractive work-up gave an isolated yield of 96% and >95% purity according to H¹-NMR (FIG. 1c).

FIG. 1a-c show the ¹H-NMR spectra obtained from the crude product (table 6, entry 1, 3 and 11, respectively). These spectra demonstrate the influence of Fe(acac)₃ and NMP in the cross-coupling reaction. In FIG. 1a is shown the ¹H-NMR of the crude product in absence of Fe(acac)₃ (table 6, entry 1). Running the reaction in the presence of Fe(acac)₃ (FIG. 1b), but without NMP did not improve the outcome (table 6, entry 3). In the presence of NMP (table 6, entry 11) the reaction was clean and high yielding (FIG. 1c). Repeating the uncatalysed reaction but using the THF-NMP solvent mixture (entry 2) gave a lower yield (20%) than using THF as the only solvent. This demonstrates the necessity of using iron salt and solvent additive to obtain a high yielding and rapid reaction. Likewise, FeCl₃ was shown to be just as efficient (entry 12) giving a yield of 95% (5 min).

Iron- and manganese salts proved to be superior as catalysts in the cross-coupling of imidoyl chloride 35 with butylmagnesium chloride. These applications were distinguished by exceptionally high reaction rates and by the low cost, ready availability and benign character of the applied salts.

Optimization of the Iron-catalyzed Cross-coupling

Organomagnesium reagents are highly reactive towards several functional groups and issues with functional group selectivity are often seen. It is, therefore, desirable to use less reactive reagents in order to extent the scope of the iron-catalyzed cross-coupling. We decided to use imidoyl chloride 35 as our primary test compound in combination with various organometallic reagents applying Fe(acac)₃. All experiments were carried out in a THF/NMP solvent mixture using 0.20 mmol imidoyl chloride 35, 0.40 mmol organometallic reagent and 5 mol % Fe(acac)₃ at ambient temperature (Scheme 9).

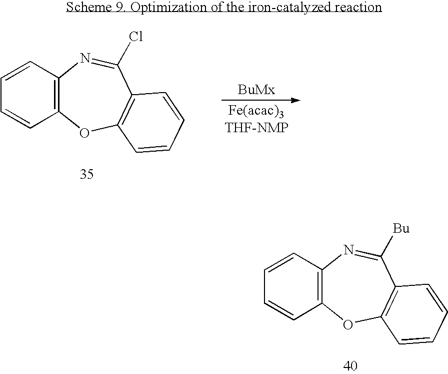

The organometallic reagents were either commercially available or prepared according to literature procedure.

TABLE 7
Screening of organometallic reagents

| Entry | RMX | Temp. (° C.) | Reac. time | Yield^{a} (%) |
|---|---|---|---|---|
| 1 | BuMgCl | 25 | 5 min | 95 |
| 2 | BuCu(CN)ZnBr | 60 | 24 h | 0 |
| 3 | BuCu(CN)MgCl | 25 | 5 min | 96 |
| 4 | BuZnBr | 60 | 24 h | 0 |
| 5 | Bu₂Zn | 60 | 24 h | 0 |
| 6 | BuMn(Cl₂)MgCl | 25 | 5 min | 95 |

^{a}¹H-NMR yields based on toluene as an internal standard.

As seen in table 7 a test-reaction was first conducted employing butylmagnesium chloride, which gave a rapid reaction and high yield (entry 1) as previously described. As illustrated by entry 2 and 3 the organometallic reagent was crucial for the outcome of the reaction. When the organocopper reagent was done by transmetallation from the corresponding organozinc halide, no reaction occurred and the starting material was recovered (entry 2). Even when the reaction was performed in refluxing THF for 24 hours, no product was detected. In contrast, organocopper reagent prepared from transmetallation from the corresponding Grignard reagent gave a rapid and high yielding reaction (entry 3). According to Knochel, the derived copper reagent is better represented as RCu(CN)MgCl and RCu(CN)ZnBr, respectively. Sapountzis, I.; Lin, W.; Kofink, C.; Despotopoulou, C. and Knochel, P.; Angew. Chem. 2005, 44, 1654-1657. The distinct difference in reactivity between the two organocopper reagents can therefore be explained by formation of different complexes coordinating copper-magnesium or copper-zinc atoms. This indicates that the presence of magnesium atoms in the organometallic reagent somehow is important for the catalytic process of iron. This was further emphasized when zinc reagents were applied, which resulted in the return of starting material (entry 4 and 5). These observations are consistent with results previously reported by Fürstner, who claim that organocopper and even organozinc do not engender iron-catalyzed cross-coupling under the conditions shown in Scheme 8. Fürstner, A. and Martin, R.; Chemistry Lett. 2005, 34, 624-629. Use of organo manganese proved also to be highly efficient (entry 6).

The presence of Grignard reagent was important for a successful cross-coupling of imidoyl chloride 35 probably due to the high reducing ability of organomagnesium halides. Hypothetically it should therefore be possible to initiate the catalytic cycle by small amounts of Grignard reagent and thereby be able to use organometallic reagents, which normally do not have the reductive power to reduce the iron precatalyst.

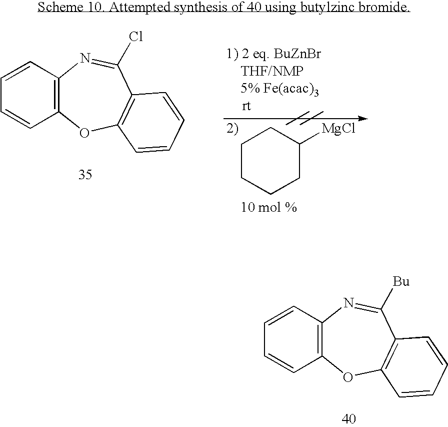

As shown in Scheme 10, 10 mol % of cyclohexylmagnesium chloride was added to reduce the iron catalyst and thereby initiate the catalytic cycle. With two equivalents of butylzinc bromide already present in the reaction mixture we anticipated that this reagent would enter the catalytic cycle when the Grignard reagent was consumed. No coupling product between the imidoyl chloride 35 and the zinc reagent was obtained with starting material being recovered. Minor amounts of the cyclohexyl coupling product and the cyclohexyl dimer confirmed that the reduction of the iron catalyst had taken place. However, for some reason, currently unknown, the organozinc halide did not enter the catalytic cycle.

In order to explore the scope and limitations of Grignard reagents in the iron-catalyzed cross-coupling of imidoyl chlorides, several different alkyl- and arylmagnesium reagents were explored (Scheme 11).

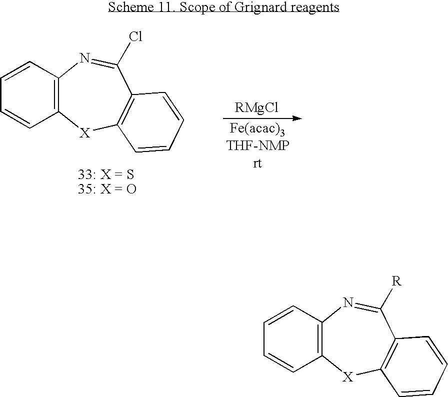

Two imidoyl chlorides 33 and 35 were used in combination with organomagnesium halides in the cross-coupling reactions similar to the previously defined evaluation reaction (Scheme 9). Unless otherwise mentioned, the experiments were conducted employing two equivalent Grignard reagents with a reaction time of 5 min.

TABLE 8
Scope of Grignard reagents

| Entry | RMX | Product | Yield^{a} (%) |
|---|---|---|---|
| 1 | BuMgCl | 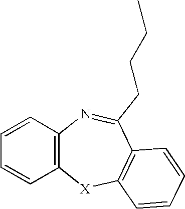 | 40: 96 (X = O) 43: 93 (X = S) |
| 2 | C₆H₁₁MgCl | 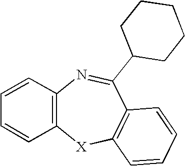 | 44: 93 (X = O) 45: 89 (X = S) |
| 3 | tBuMgCl | 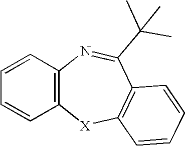 | 46: 27 (X = O) |
| 4 | 1,3-dioxane-2- ethylmagnesium chloride | 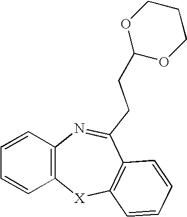 | 47: 95 (X = O) 48: 86 (X = S) |
| 5 | Me₃SiCH₂MgCl | 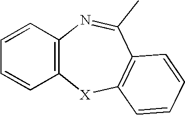 | 49: 72 (X = O) |
| 6 | MeMgCl | 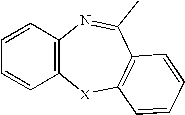 | 49: 17 (X = O) |
| 7^{b} | PhMgCl | 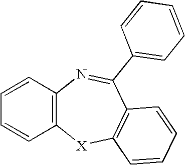 | 50: 51 (X = O) |

^{a}Isolated yields.
^{b}Six equivalent of phenylmagnesium chloride was employed to obtain complete conversion of starting material. 30 mm reaction time.

As exemplified in table 8, butylmagnesium choride gave excellent yields (>93%) in the iron-catalyzed cross-coupling reaction with imidoyl chloride 33 and 35 (entry 1). Likewise, introduction of the more sterically hindered cyclohexylmagnesium chloride (entry 2) gave high isolated yields of 44 (93%) and 45 (89%). Iron-catalyzed addition of a more sterically demanding sec-alkylmagnesium halide has been reported, however, use of special iron complexes was necessary. Fürstner, A.; Leitner, A.; Méndez, M. and Krause, H.; J. Am. Chem. Soc. 2002, 13856-13863. Although the yield was low (27%) it was quite remarkable that the crowded tert-butylmagnesium chloride (entry 3) still gave product. Functionalized alkylmagnesium reagents were of interest since the introduced group could be good starting point for further derivatization. A Grignard reagent including some oxygen functionalities in form of an acetal (entry 4) was well-tolerated affording yields of 47 (95%) and 48 (86%), respectively. The (trimethylsilyl)methylmagnesium chloride (entry 5) give the desilylated methyl product as the sole product in 72% yield. No silylated product was detected in the reaction mixture, according to GC/MS analysis. Me₃SiCH₂MgCl has successfully been used in this type of transformations using alkenyl triflate without elimination of the silyl group. Scheiper, Bodo; Bonnekessel, M.; Krause, H. and Fürstner, A.; J. Org. Chem. 2004, 69, 3943-3949. The stabilizing effect by the trimethylsilyl group on the β carbon in imidoyl chloride 35 combined with the presence of a nearby nitrogen, could explain the immediate formation of the desilylated methyl product (entry 5). Hence, the combined stabilization could provide the trimethylsilyl group to be eliminated more easily. As seen in entry 6, methylmagnesium bromide gave a low yield. Scheiper, Bodo; Bonnekessel, M.; Krause, H. and Fürstner, A.; J. Org. Chem. 2004, 69, 3943-3949. Since Me₃SiCH₂MgCl gave high yields of the methylated product, the use of this reagent could be a way to introduce a methyl group. Addition of PhMgCl gave moderate yield (51%, entry 7). It was necessary to use six equivalents of the Grignard reagent to obtain complete conversion of the starting material. This is explained by a competing homo-coupling of the aryl reagent producing the biaryl. A large amount of the biaryl was also detected in the crude product by GC-MS analysis. The problem with homo-coupling, when arylmagnesium halides are used in iron-catalyzed cross-coupling, has been demonstrated before and constitutes a limitation of this method. Fürstner, A.; Leitner, A.; Méndez, M. and Krause, H.; J. Am. Chem. Soc. 2002, 13856-13863. To further optimize the formation of the Csp²-Csp² carbon bond (table 6, entry 7), the Fe(acac)₃ catalyzed cross-coupling of 35 with phenylmagnesium chloride was examined (Scheme 12).

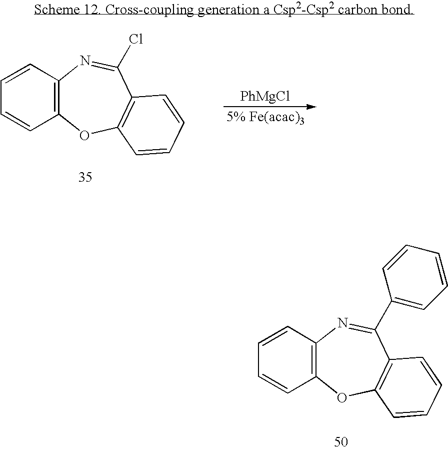

All reactions were conducted using 0.20 mmol imidoyl chloride 35, 5 mol % Fe(acac)₃ and six equivalent phenylmagnesium chloride. Unless otherwise mentioned the reactions were carried out at room temperature.

TABLE 9
Optimerization of the iron-catalyzed cross-coupling
using arylmagnesium halides

| | | | D. Reac. | E. Yield^{a} |
|---|---|---|---|---|
| A. Entry | B. Catalyst | C. Solvent | time | (%) |
| F. 1 | None | THF | G. 18 h | H. 22 |
| I. 2 | Fe(acac)₃ | THF | J. 30 min | K. 51 |
| | | (+NMP) | | |
| L. 3 | Fe(acac)₃ | THF | M. 30 min | N. 55 |
| O. 4 | Fe(acac)₃ | Et₂O | P. 30 min | Q. 41 |
| R. 5 | Fe(acac)₃ | Et₂O^{b} | S. 30 min | T. 45 |

^{a}¹H-NMR yields based on toluene as an internal standard.
^{b}The reaction was carried out in refluxing Et₂O.

As exemplified in table 9, the uncatalysed reaction (entry 1) was first conducted giving a low yield of 22% after an 18 h reaction time with starting material being recovered. Running the reaction in a THF/NMP solvent mixture in the presence of 5 mol % Fe(acac)₃ (entry 2) increased the yield (51%) as presented in table 9 (entry 7) with a decrease in reaction time (30 min). However, in this reaction NMP did not have any effect on the iron-catalyzed reaction in contrast to the previous experiments. Hence, the reaction in THF gave a similar result (55%, entry 3). Use of diethyl ether as a solvent in place of THF has been reported to improve the yield in cross-coupling of aryl Grignard. Nagano, T. and Hayashi, T. Organic Letters 2004, 6, 1297-1299. However, in this case the yield was still moderate.

Synthesis of Carbon Analogue of Clozapine

In the light of the positive results obtained in the screening and optimerization of the cross-coupling of the tricyclic imidoyl chlorides we decided to focus on the synthesis of clozapine analogues. The N-methyl piperidine magnesium chloride was prepared according to literature procedure and applied in the optimized iron-catalyze cross-coupling with the imidoyl chlorides 1, 34 and 36 (Scheme 13). Engelhardt, E. L.; Zell, H. C.; Saari, W. S.; Christy, M. E. and Dylion Colton, C.; J. Med. Chem. 1965, 8, 829-835.

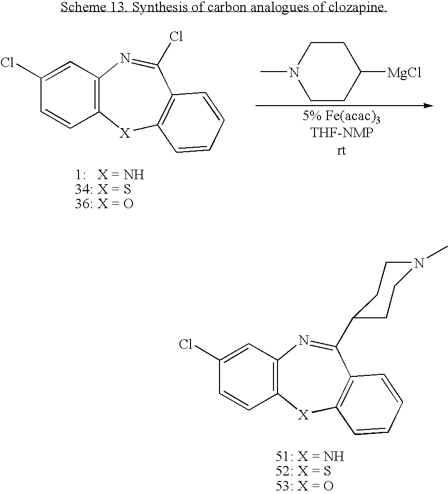

The reactions were conducted similar to the previously defined (Scheme 11).

TABLE 10
Results from the synthesis of clozapine analogues.

| Entry | X | React. time (min) | Yield^{a} (%) |
|---|---|---|---|
| 1 | NH | 5 | 82 |
| 2 | O | 5 | 71 |
| 3 | S | 5 | 86 |

^{a}Isolated yields.

As shown in table 10, the optimized iron-catalyzed procedure was a convenient way to obtain the carbon analogues in good to excellent yields (71-86%). Importantly, even the unprotected imidoyl chloride 1 (entry 1) containing an N—H bond undergoes efficient cross-coupling, although an extra equivalent of the Grignard reagent is necessary to obtain complete conversion.

Application of Functionalized Aryl Grignard Reagents

The high reactivity of the C—MgX bond restricts the scope of the iron-catalyzed cross-coupling methodology. However, the rapidly growing number of functionalized Grignard reagents, which are obtained and stable at subzero temperatures could circumvent the problems associated with group selectivity of Grignard reagents. In addition, iron-catalyzed cross-coupling reactions have been reported to occur within minutes at very mild conditions (−60° C.) leading to almost quantitative yields. A decrease of the temperature and its possible affect on the iron-catalyzed cross-coupling of imidoyl chlorides (table 11) was investigated.

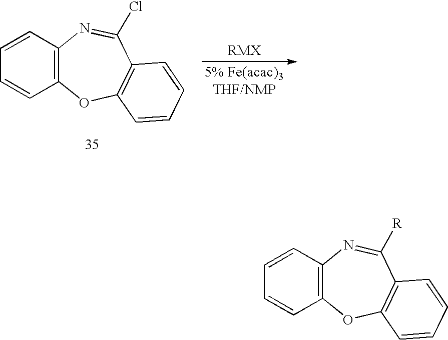

TABLE 11
Optimerization of the iron-catalyzed reaction.

| Entry | RMX | Temp. (° C.) | Reac. time (min) | Yield^{a} (%) |
|---|---|---|---|---|
| 1^{b} | BuMgCl | −40 | 60 | 0 |
| 2 | BuMgCl | −40 | 5 | 95 |
| 3 | BuMgCl | −78 | 5 | 95 |
| 4 | PhMgCl | −40 | 30 | 49 |

^{a}¹H-NMR yields based on toluene as an internal standard.
^{b}The reaction was carried out in THF in absence of Fe(acac)₃.

As shown in table 11, the temperature studies showed the iron-catalyzed reaction to be extremely rapid and efficient even at low temperatures. A control experiment running the reaction at −40° C. in absence of Fe(acac)₃ returned quantitatively the starting material after a 60 min reaction time (entry 1). However, upon addition of 5 mol % Fe(acac)₃ the reaction was completed within 5 min to give the butylated product in 95% yield (entry 2). Further decrease in temperature (−78° C.) did not have an effect on the outcome (entry 3). Introducing a phenyl group into the imidoyl chloride 35 (entry 4) using the conditions described in Scheme 12 gave a moderate yield of 49%. Again an excess of the Grignard reagent (six equivalents) was necessary to obtain full conversion of the starting material due to the extensive formation of biaryl. Functionalized aryl Grignard reagents were used in order to extend the scope of the iron-catalysed procedure (Scheme 14).

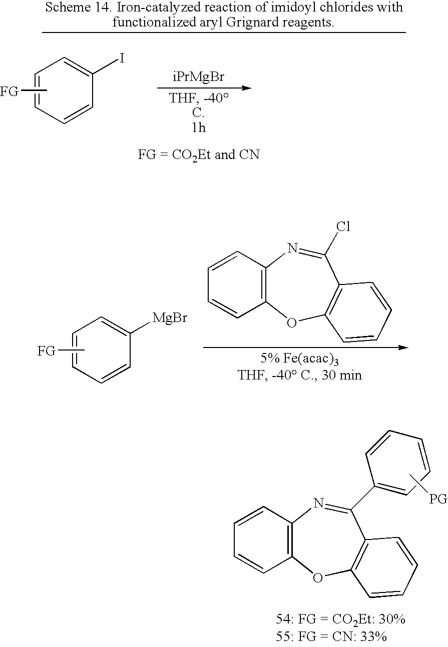

As demonstrated in Scheme 14, different aryl iodide reagents were smoothly converted into the corresponding aryl magnesium halide by a magnesium-iodide exchange with iPrMgBr at −40° C. Tucker, C. E.; Majid, T. N. and Knochel, P.; J. Am. Chem. Soc. 1992, 114, 3983-3985. The resulting Grignard reagents were coupled with 35 under conditions previously defined at −40° C. (table 11). However, the yields obtained were low (25-30%) compared to the moderate yield in the test reaction using phenylmagnesium chloride. Six equivalents of Grignard reagents were, in this case, not sufficient to obtain full conversion of the starting material due to extensive homo-coupling of the aryl reagent. An attempt to slowly add the aryl Grignard reagent by syringe pump over a period of 30 min, in order to maintain a low concentration did not suppress the homo-coupling.

Application of Functionalized Imidoyl Chlorides

The inherent problem with formation of biaryls seems to be difficult to avoid. A sensitive functionality in the imidoyl chloride was introduced to demonstrate that it is in fact possible to obtain group selectivity in iron-catalyzed cross-coupling of imidoyl chlorides. Ester 61 was synthesized according to the procedure depicted in Scheme 15.

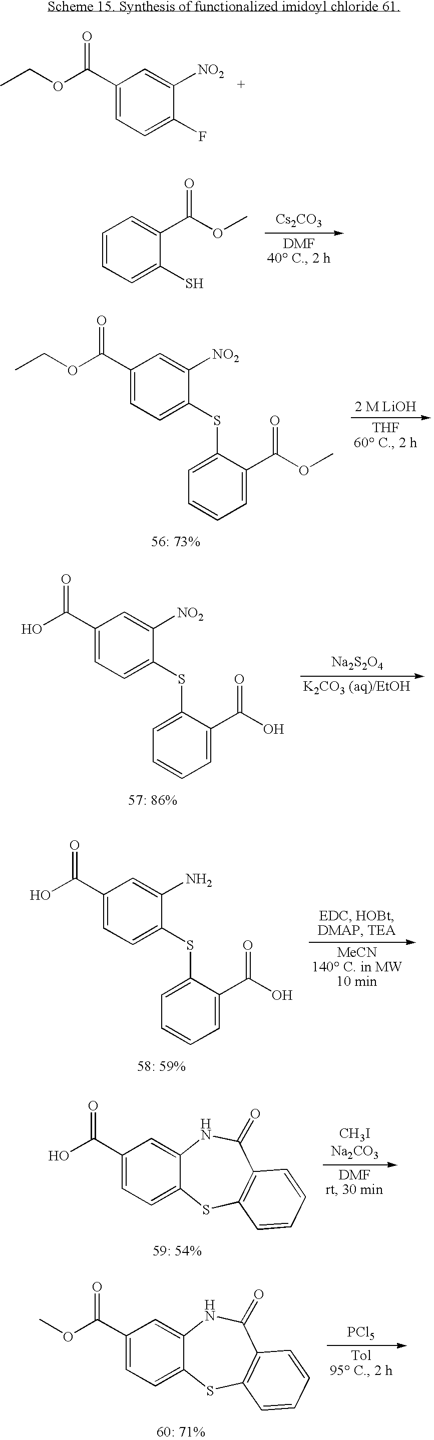

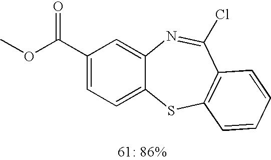

The previously described procedure B, was used in the synthesis of lactam 59. However, in the ECDI coupling an extra equivalent of the coupling agents was used, due to the presence of two carboxylic acids in intermediate 58. The carboxylic acid 59 was converted to the methyl ester 60 upon treatment with methyl iodide in combination with Na₂CO₃ in DMF. Imidoyl chloride 61 was then obtained by reacting lactam 60 with PCl₅ in refluxing toluene for two hours. No break down of the methyl ester functionality was detected. However, an unidentified by-product was formed (5%, seen in LC-MS), which could not be separated from the product by column chromatography. (Formation of the functionalized imidoyl chloride by thionyl cloride gave a purity of 99% according to LC-MS analysis.) The functionalized imidoyl chloride 61 was then applied in the iron-catalyzed cross-coupling reaction with butylmagnesium chloride using the optimized conditions (Scheme 16).

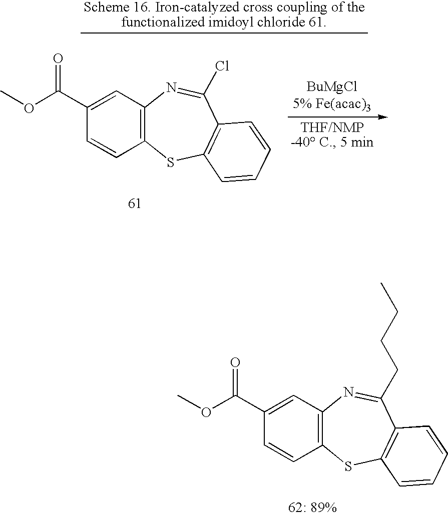

As shown in Scheme 16, the ester functionalised imidoyl chloride 61 reacted smoothly with butylmagnesium chloride in the presence of 5 mol % Fe(acac)₃ at −40° C. giving a good yield of 62 (89%). No products from the anticipated competing addition to the ester were isolated. Performing the reaction in the absence of Fe(acac)₃ at −10° C. was detrimental giving complex product mixtures. To further investigate the chemoselectivity of the iron-catalyzed procedure, a Weinreb amide functionalized imidoyl chloride was synthesized from 63 (Scheme 17).

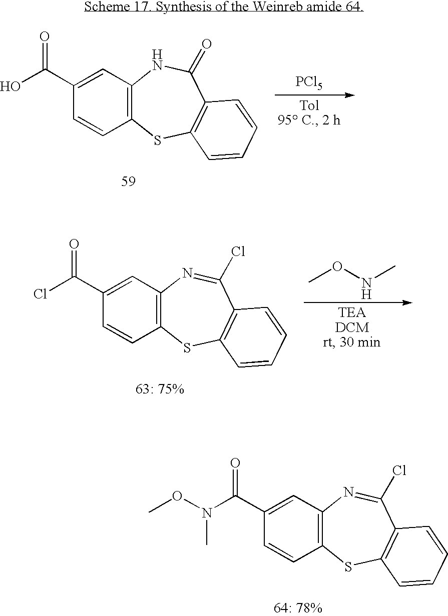

Reacting 59 with PCl₅ prepared the functionalized imidoyl chloride 63 in refluxing toluene. Addition of N,O-dimethylhydroxylamine at room temperature gave exclusively Weinreb amide 64 in good yield. No addition to the imidoyl chloride moiety in 64 was observed further demonstrating the significant difference in reactivity between the acyl chloride and the imidoyl chloride functionalities. The imidoyl chloride 64 was then reacted with butylmagnesium chloride as demonstrated in Scheme 18.

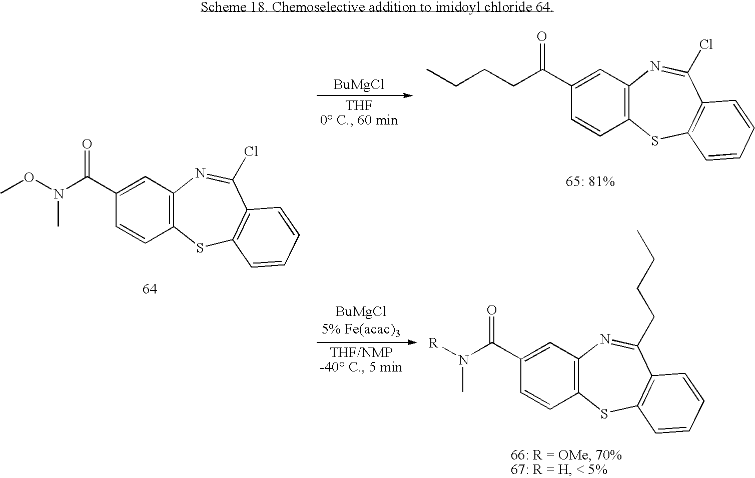

As seen in Scheme 18, it was possible to chemoselectively substitute either the chlorine atom or add to the Weinreb amide. Selective addition of butylmagnesium chloride to the Weinreb amide moiety of 64 was achieved in absence of iron catalyst at 0° C., which gave 81% of 65. Running the reaction at −40° C. in presence of 5 mol % Fe(acac)₃ gave upon addition of one equivalent butylmagnesium chloride 66 in 70% yield with minor formation of the demethoxylated methyl amide 67. Elimination of OCH₃ has been reported by addition of Grignard to Weinreb amides at 0° C. Lubell, W. D.; Jamison, T. F. and Rapoport, H.; J. Org. Chem. 1990, 55, 3511-3522; Sibi, M. P.; Marvin, M. and Sharma, R.; J. Org. Chem. 1995, 60, 5016-5023. Although, in our case, the N—O cleavage was performed under considerably milder conditions (−78° C.) and complete demethoxylation could be obtained by addition of two equivalents butylmagnesium chloride, which gave 67 in good yields considering the tandem transformation (Scheme 19).

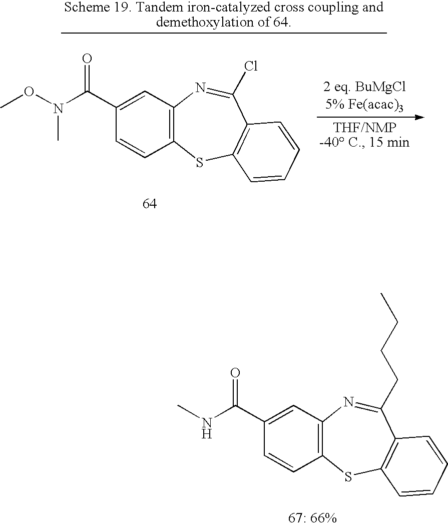

Repeating the reaction in absence of the Fe(acac)₃ at −40° C. returned the starting material indicating that the catalyst play a critical role in the demethoxylation process. Imidoyl chloride 65 was finally applied in the iron-catalyzed cross-coupling with cyclohexylmagnesium chloride (Scheme 20).

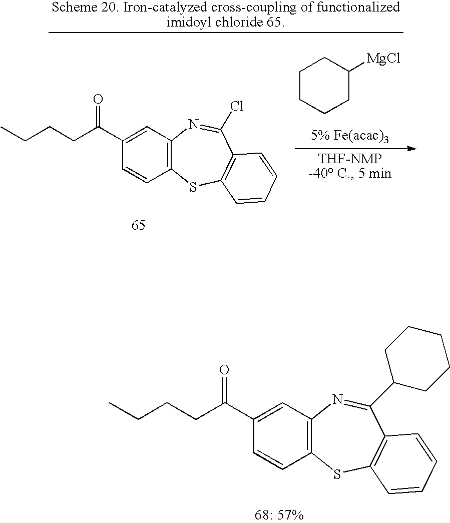

Again a high compatibility of functionalities was demonstrated leaving the ketone moiety intact. Likewise was the Weinreb amide product 66 reacted with cyclohexylmagnesium chloride (Scheme 21).

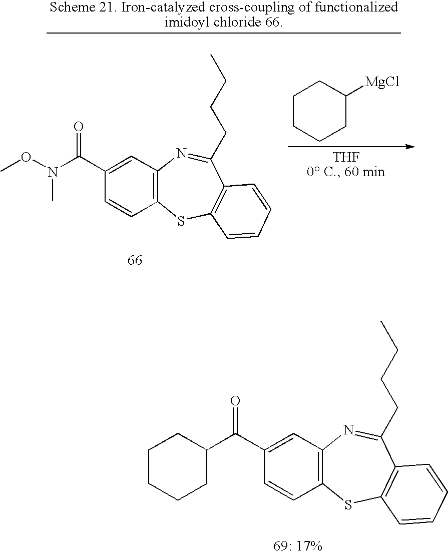

The lower yield in this reaction compared to the addition of butylmagnesium chloride is probably explained by increased steric hindrance of the cyclohexyl group combined with the fact that this reaction was conducted on a 0.08 mmol (26 mg) scale. On the basis of the above experiments a one-pot procedure was conducted (Scheme 22).

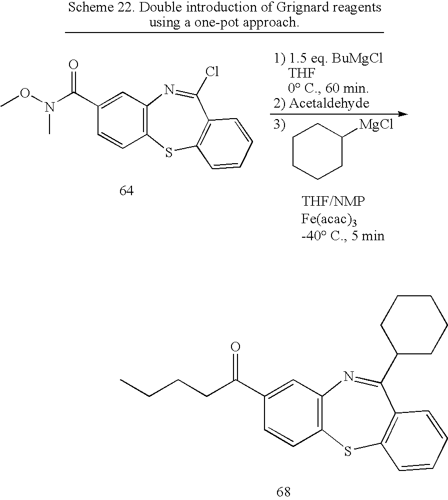

Butylmagnesium chloride (1.5 equivalent) was added at 0° C. to 64 in THF, which gave exclusively addition to the Weinreb amide moiety. The excess of the butylmagnesium chloride was quenched by addition of one equivalent of acetaldehyde before the addition of 5 mol % Fe(acac)₃ dissolved in THF/NMP. The resulting solution was then cooled to −40° C. and cyclohexylmagnesium chloride was added. Preliminary result shows that 68 is formed (confirmed by GC-MS).

General synthetic discussion regarding the process and synthesis of compounds of this invention are shown in paragraph below including table 13 and Schemes 23-25. The routes shown are illustrative only and are not intended, nor are they to be construed, to limit the scope of this invention in any manner whatsoever. Those skilled in the art will be able to recognize modifications of the disclosed synthetic methodology and to devise alternate applications based on the disclosures herein; all such modifications and alternate applications are within the scope of this invention.

In the initial studies, n-BuMgCl was added to imidoyl chloride 70 generated from the corresponding lactam. The uncatalyzed reaction gave low yields, 42% of 2 at ambient temperature in THF with a 30 min reaction time. Lower temperatures gave longer reaction times and at −40° C. the reaction quantitatively returned the starting material (1). Adding 5 mol % of Fe(acac)₃ at rt. gave a fast reaction and the imidoyl chloride was consumed within 5 min, disappointingly the yield (22%) was lower than the uncatalyzed reactions (entrie 2 and 3, Table 13). However, applying a THF-NMP solvent mixture produced an excellent isolated yield of 96% and a 5 min reaction time were achieved (entry 4, Table 13). A simple extractive work-up gave 71 in >95% purity. See Examples for H¹-NMR comparing entries 1, 2 and 3 after the extractive work-up. Even at −78 C using these conditions the reaction was finished within 5 min, with an isolated yield of 94%. The iron-catalyst FeCl₃ and Fe(acac)₃ were interchangeable, such that at room temperature no difference in the reaction outcome was seen (entries 4 and 6, Table 13). Repeating the uncatalyzed reaction but using the THF-NMP solvent mixture gave a lower yield (<20%) than using THF as sole solvent. This showed that using an iron-catalyst resulted in high yields and fast reactions.

TABLE 13
Iron-catalysed cross-coupling of imidoyl chloride 70

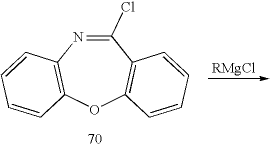

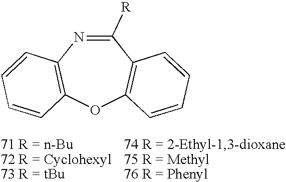

| entry | R | solvent/catalyst | temp/time | yield^{a} |
|---|---|---|---|---|
| 1 | n-Bu | THF/none | rt/30 min | 42% |
| 2 | n-Bu | THF/Fe(acac)₃ | rt/5 min | 22% |
| 3 | n-Bu | THF/Fe(acac)₃ | rt/30 min | 42% |
| 4 | n-Bu | THF-NMP/Fe(acac)₃ | rt/5 min | 96% |
| 5 | n-Bu | THF-NMP/Fe(acac)₃ | −78 C./5 | 94% |
| | | | min | |
| 6 | n-Bu | THF-NMP/FeCl₃ | rt/5 min | 96% |
| 7 | n-Bu | THF-NMP | rt/30 min | 20% |
| 8 | Cyclohexyl | THF-NMP/Fe(acac)₃ | rt/5 min | 93% |
| 9 | t-Bu | THF-NMP/Fe(acac)₃ | rt/5 min | 27% |
| 10 | 2-Ethyl-1,3 dioxane | THF-NMP/Fe(acac)₃ | rt/5 min | 95% |
| 11 | Me | THF-NMP/Fe(acac)₃ | rt/5 min | 17% |
| 12 | SiMe₄ | THF-NMP/Fe(acac)₃ | rt/5 min | (72%)^{b} |
| 13 | Ph | THF-NMP/Fe(acac)₃ | rt/5 min | 55% |

^{a}Isolated yields.
^{b}The yield in the paranthese reflects R = Me, thus the desilylated product 6.

The reaction between the more sterically demanding cyclohexyl magnesium chloride and 70, produced an excellent isolated yield (93%) of 72. Although giving a low yield (27%) it was encouraging that using the standard conditions the quite sterically encumbered t-BuMgCl still gave product 73. Including some oxygen functionalities in form of an acetal in the Grignard reagent was well tolerated (entry 10, compound 74, 95% yield). As noticed before in other iron-catalyzed reactions, the lowest alkyl nucleophile methylmagnesium bromide failed to efficiently react giving the low yield 17% of 75. The methyl equivalent (Trimethylsilyl)methyl) magnesium chloride gave 75 as the sole product in 72% yield. No silylated product was detected in the reaction mixture, according to GC/MS. Me₃SiCH₂MgCl has successfully been used in this type of transformations using alkenyl triflate without elimination of the silyl group. Furthermore, adding PhMgCl generating a sp²-sp² carbon-carbon bond gave a 55% yield of 76. In this case all the tested solvents, i.e. THF, THF-NMP and Et₂O basically gave the same yield. The lower yield is explained by a competing homocoupling of the aryl reagent producing the biaryl.

Focusing on clozapine analogs, imidoyl chlorides 77-79 were synthesized and reacted with N-methyl piperidine magnesium chloride to give azepines 80-82 in good yields (71-86%).

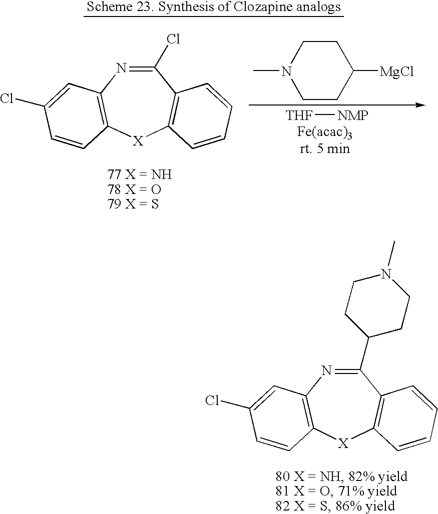

The mild reaction conditions (−78° C. and 5 min reaction time (entry 5, Table 13) indicated the possibility of having additional functionalities present during the reaction. An ester functionalized imidoyl chloride (83) was synthesized and reacted with n-butyl Grignard at −40° C. for 5 min, producing a 89% yield while leaving the ester functionality intact (See 84, Scheme 24). No products from the anticipated competing addition to the ester were isolated. Running the reaction in the absence of Fe(acac)₃ at −10° C. was detrimental, giving complex product mixtures

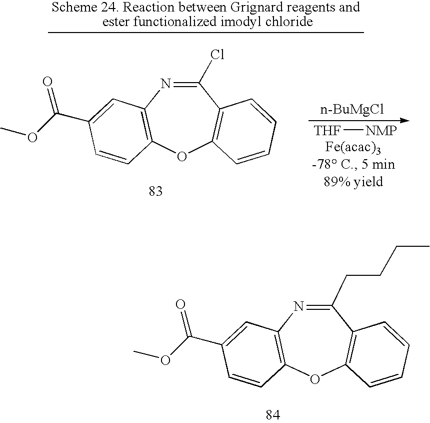

Starting with a Weinreb amide functionalized imidoyl chloride 85 made it possible to selectively substitute the chloride or Weinreb amide, using Fe(acac)₃ as the catalyst (Scheme 25). The reaction produced the product 86 in 70% yield as major product and the demethoxylated Weinreb product 87 as the minor product in 5% yield. Selective addition to the Weinreb amide (85) was achieved in 81% yield, 88, by not adding any iron-catalyst at 0° C. in THF.

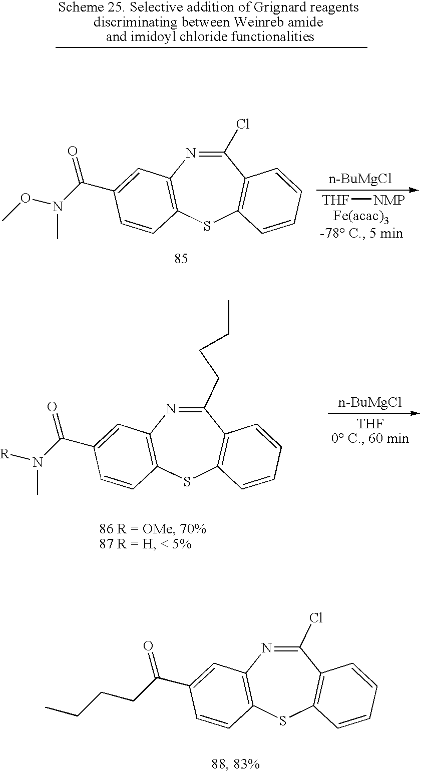

As demonstrated in Scheme 26, the iron-catalyzed reaction is not limited to cyclic compounds. Imine 89 was synthesized from amide 88 in 72% yield over two steps using standard conditions. The intermediate imidoyl chloride was concentrated at reduced pressure and used in crosss-coupling , without further purification.

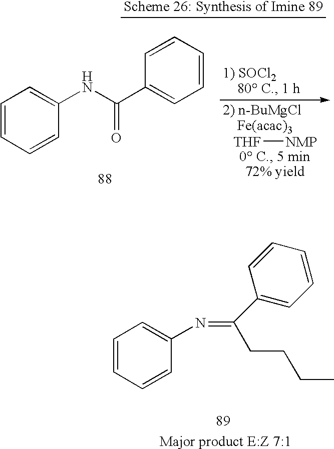

### EXAMPLES

General Conditions

All reactions involving dry solvents or sensitive reagents were performed in flame-dried glassware under a nitrogen or argon atmosphere.

Solvents

All solvents were of HPLC grade.

Reagents

1 M CuCN:2LiCl solution was prepared by drying CuCN (0.90 g, 0.01 mol) and LiCl (0.85 g, 0.02 mol) in a schlenk flask under vacuum for 1 hour at 120° C. After cooling to room temperature, dry THF (10 ml) was added and stirring was continued until the salts were dissolved.

1 M MnCl₂:2LiCl solution was prepared by drying MnCl₂ (1.26 g, 0.01 mol) and LiCl (0.85 g, 0.02 mol) in a schlenk flask under vacuum for 1 hour at 120° C. After cooling to room temperature, dry THF (10 ml) was added and stirring was continued until the salts were dissolved. Cahiez, Gérard; Luart, Denis and Lecomte, Fabien; Org. Lett.; 2004, 6, 4395-4398.

The following functionalized Grignard reagents were prepared according to literature procedures: N-methyl piperidine magnesium chloride, 4-methyl benzoate magnesium chloride and 4-benzonitrile magnesium chloride. Engelhardt, E. L.; Zell, H. C.; Saari, W. S.; Christy, M. E. and Dylion Colton, C.; J. Med. Chem. 1965, 8, 829-835; Boymond, L.; Rottländer, M.; Cahiez, G. and Knochel, P.; Angew. Chem. 1998, 37, 1701.

Content Determination of Organometallic Reagents

Organomagnesium solutions were titrated using a method reported by Lin, H. S. and Paquette, L. A.; Synth. Commun. 1994, 24, 2503.

Chromatography

Thin layer chromatography (TLC) was performed using aluminium plates coated with SiO₂. The plates were viewed under UV light and/or by treatment of the TLC plate with solution of KMnO₄.

Preparative TLC: PSC plates 20×20 cm. Silica gel 69 F₂₅₄, 0.5 mm.

Column chromatography was performed using SiO₂ 60 (0.040-0.063) from Merck.

Analytical Data

NMR spectra were recorded on a Varian mercury-400 VX. Chemical shifts are reported as δ-values in ppm relative to the solvent peak: CDCl3 (δH: 7.26, δC: 77.16), methanol-d4 (δH: 3.31, δC: 49.00), DMSO-d6: (δH: 2.50, δC: 39.52), acetone-d6: (δH: 2.05, δC: 29.84). For the characterization of the observed signal multiplicities the following abbreviations were applied: s (singlet), d (doublet), t (triplet), m (multiplet) as well as br (broad).

### Example 1

### Synthesis of 4-methylpiperidine magnesium chloride

A dried, three-necked flash equipped with argon inlet, a dropping funnel and a thermometer was charged with magnesium turnings (2.52 g, 0.10 mol), which before use had been washed with 0.01 M H₂SO₄ (aq.) and dried. A small amount of dry THF was added to cover the magnesium. A crystal of iodine was added followed by dibromoethane. When the vigorous reaction had subsided, a solution of distilled 4-chloro-1-methylpiperidine (9.20 g, 0.07 mol) in THF (70 mL) was added dropwise. When the addition was complete, the reaction mixture was heated to refluxing with stirring for 1 h. The reaction mixture was 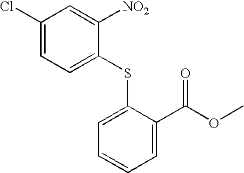 then allowed to cool to room temperature. Full conversion was confirmed by hydrolysis (GC) and generation of Grignard reagent by iodolysis (GC).

### Example 2

### Synthesis of 4-methyl benzoate magnesium chloride

A dry 10 mL Schlenk flask was charged under argon with methyl 4-iodobenzoat (84 mg, 0.32 mmol, 1 eq.). Dry THF (0.32 mL) was added, and the solution was cooled to −25° C., then isopropylmagnesium chloride (0.17 mL, 2.0 M in THF, 1.05 eq.) was added slowly over a 5 min. periode, maintaining the temperature below −20° C. On completion of the addition, the reaction mixture was stirred at −20° C. for 0.5 hour. Full conversion was confirmed by hydrolysis (GC) and generation of Grignard reagent by iodolysis (GC).

### Example 3

### Synthesis of 4-benzonitrile magnesium chloride

A dry 10 mL Schlenk flask was charged under argon with 4-iodobenzonitrile (343.8 mg, 1.50 mmol, 1 eq.). Dry THF (1.50 mL) was added, and the solution was cooled to −25° C., then isopropylmagnesium chloride (0.88 mL, 2.0 M in THF, 1.05 eq.) was added slowly over a 5 min. periode, maintaining the temperature below −20° C. On completion of the addition, the reaction mixture was stirred at −20° C. for 0.5 hour. Full conversion was confirmed by hydrolysis (GC) and generation of Grignard reagent by iodolysis (GC).

### Example 4

### Methyl 2-(4-chloro-2-nitrophenylthio)benzoate (1) 0480

A solution of 4-chloro-1-fluoro-2-nitrobenzene (2.00 g, 0.011 mol) and methyl 2-mercaptobenzoate (3.13 mL, 0.023 mol) in DMF (25 mL) was added Cs₂CO₃ (7.43 g, 0.023 mol) and the resulting mixture was stirred for 2 hours at 40° C. The reaction mixture was cooled to room temperature, diluted with DCM, washed with water, dried (Na₂SO₃), filtered and evaporated to give crude product. Purification by flash chromatography (ethyl acetate/heptane 1:4) gave 2.6 g (73%) of the title compound as a yellow solid. ¹H NMR (400 MHz, CDCl₃): δ 8.15 (1H, m ), 7.94 (1H, m), 7.50 (3H, m), 7.34 (1H, ddd, J=8.8, 2.8, 0.4 Hz), 6.95 (1H, d, J=8.4 Hz), 3.82 (3H, s).

### Example 5

### Methyl 2-(4.chloro-2-nitrophenoxy)benzoate (2)

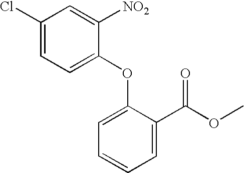

A solution of 4-chloro-1-fluoro-2-nitrobenzene (2.00 g, 0.011 mol) and methyl salicylate (2.92 mL, 0.023 mol) in DMF (25 mL) was added Cs₂CO₃ (7.43 g, 0.023 mol) and the resulting mixture was stirred for 2 hours at 40° C. The reaction mixture was then cooled to room temperature, diluted with DCM, washed with water, dried (Na₂SO₃), filtered and evaporated to give crude product. Purification by flash chromatography (ethyl acetate/heptane 1:4) gave 2.80 g (80%) of the title compound as a yellow solid. ¹H NMR (400 MHz, CDCl₃): δ 8.01 (1H, dd, J=8.8, 1.6 Hz), 7.96 (1H, d, J=2.4 Hz), 7.58 (1H, ddd, J=9.2, 7.6, 2.0 Hz), 7.39 (1H, dd, J=8.8, 2.4 Hz), 7.34 (1H, dt, J=7.6, 1.2 Hz), 7.13 (1H, dd, J=8.4, 1.2 Hz), 6.74 (1H, d, J=9.2 Hz), 3.72 (3H, s).¹³C NMR (100 MHz, CDCl₃): δ 165.2, 153.9, 150.6, 134.6, 134.4, 132.8, 127.7, 126.0, 125.8, 123.8, 122.6, 119.6, 118.3, 52.1.

### Example 6

### 2-(2-Nitro-phenylsulfanyl)benzoic acid methyl ester (3)

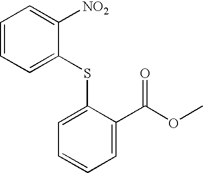

A solution of 1-fluoro-2-nitrobenzene (2.50 g, 17.7 mmol mol) and methyl 2-mercaptobenzoate (4.86 mL, 35.4 mmol) in DMF (20 mL) was added Cs₂CO₃ (11.55 g, 35.4 mmol) and the resulting mixture was stirred for 2 hours at 40° C. The reaction mixture was then cooled to room temperature, diluted with DCM, washed with water, dried (Na₂SO₃), filtered and evaporated to give crude product. Purification by flash chromatography (ethyl acetate/heptane 1:4) gave 2.3 g (45%) of the title compound as a yellow solid. ¹H NMR (400 MHz, CDCl₃): δ 8.14 (1H, dd, J=8.0, 1.2 Hz), 7.94-7.91 (1H, m), 7.51-7.45 (3H, m), 7.41-7.37 (1H, m), 7.32-7.28 (1H, m), 7.04 (1H, dd, J=8.0, 1.2 Hz), 3.81 (3H, s). ¹³C NMR (100 MHz, CDCl₃): δ 167.1, 147.4, 136.7, 135.9, 134.9, 133.8, 133.4, 132.7, 131.4, 131.2, 129.1, 126.5, 125.6, 52.6.

### Example 7

### 2-(2-nitro-phenylamino)-benzoic acid (4)

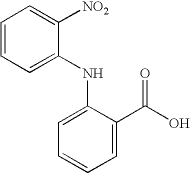

A solution of 1-fluoro-2-nitrobenzene (6.90 mL, 65.6 mmol mol) and 2-amino-benzoic acid (3.0 g, 21.9 mmol) in DMF (50 mL) was added Cs₂CO₃ (9.07 g, 65.6 mmol) and the resulting mixture was stirred at 140° C. for 2 hours. The reaction was then cooled to room temperature and diluted with EtOAc and H₂O. The resulting solution was then acidified with HCl (2M) and the organic layer was separated, washed with water, brine, dried (Na₂SO₃), filtered and evaporated to give crude product. Purification by recrystallization from MeOH gave 4.5 g (79%) of the title compound as a yellow powder. ¹H NMR (400 MHz, CDCl₃): δ 7.96-7.92 (1H, m), 7.26-7.21 (1H, m), 7.80-7.09 (2H, m), 6.88-6.84 (1H, m), 6.74-6.68 (1H, m), 6.66-6.58 (2H, m).

### Example 8

### Typical Procedure for the Hydrolysis

A solution of the appropriate methyl ester (1 eq.) in THF and 2 M LiOH (aq.) (5 eq.) was stirred at 60° C. for 2 hours, then allowed to cool to room temperature. THF was removed at reduced pressure and the aqueous mixture was acidified with HCl (2M) until pH 2. The precipitation was filtered off, washed with 0.1 M NaOH solution and finally dried to give crude product, which was used without further purification.

### Example 9

### Typical Procedure for the Reduction

A solution of the appropriate nitro benzene (1 eq.) in 2 M K₂CO₃ (aq.) and ethanol was added Na₂S₂O₄ (5 eq.). The reaction was stirred at room temperature for 10 min. EtOH was then removed at reduced pressure and the resulting aqueous mixture was acidified with HCl (2M) and poured into ethyl acetate. The organic layer was separated, washed with water, brine, dried (Na₂SO₃), filtered and evaporated to give crude product, which was used without further purification.

### Example 10

### Typical Procedures for the EDC Coupling

Method A: A solution of the appropriate amino acid (1 eq.), EDC (1.5 eq.), HOBt (1.5 eq.), DMAP (0.01 eq.) and TEA (4.5 eq.) in MeCN was heated in microwave at 140° C. for 10 min. The reaction mixture was cooled to room temperature, diluted with H₂O and acidified with HCl (2M) until pH 2. The precipitation was filtered off, washed with 0.1 M NaOH solution and finally dried to give crude product, which was used without further purification.

Method B: A solution of the appropriate amino acid (1 eq.) in DCM was cooled to 0° C. and EDC (1.5 eq.) was added. The reaction mixture was allowed to warm up to room temperature and stirred for 1 hour. The resulting precipitation was filtered off, washed with 0.1 M NaOH solution and finally dried to give crude product, which was used without further purification.

### Example 11

### 8-chloro-10H-dibenzo[b,f][1,4]thiazepin-11-one (5)

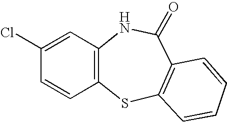

The typical procedure for the Hydrolysis was applied and the following reagents were employed: Methyl 2-(4-chloro-2-nitrophenylthio)benzoate (2.6 g, 8.0 mmol), 2 M LiOH (aq.) (13 mL) and THF (13 mL). This afforded 2.3 g (93%), which was used without further purification in the reduction step. The typical procedure for the Reduction was applied and the following reagents were employed: 2-(4-chloro-2-nitrophenylthio)benzoic acid (2.3 g, 0.007 mol), 2 M K₂CO₃ (aq.) (20 mL), Na₂S₂O₄ (6.44 g, 0.037 mol). This afforded 1.2 g (61%), which was used in the EDC coupling without further purification. The typical procedure Method A for the EDC coupling was applied to form the title compound and the following reagents were employed: 2-(2-amino-4-chlorophenylthio)benzoic acid (500 mg, 1.78 mmol), EDC (516 mg, 2.7 mmol), HOBt (365 mg, 2.7 mmol), DMAP (2.2 mg, 0.818 mmol), TEA (1.1 mL, 8.0 mmol), MeCN (1.8 mL). This afforded 238 mg (51%) of the title compound as a white powder, which was sufficiently pure to be used in the next step without further purifications. ¹H NMR (400 MHz, DMSO-d₆): δ 10.72 (1H, bs), 7.68 (1H, m), 7.51 (4H, m), 7.28 (1H, d, J=2.4 Hz), 7.20 (1H, dd, J=8.4, 2.4 Hz). ¹³C NMR (100 MHz, DMSO d₆):

### Example 12

### 8-chloro-10H-dibenzo[b,l][1,4]oxazepin-11-one (6)

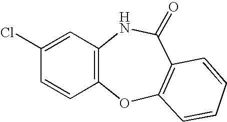

The typical procedure for the Hydrolysis was applied and the following reagents were employed: Methyl 2-(4.chloro-2-nitrophenoxy)benzoate (2.80 g, 9.55 mmol), 2 M LiOH (aq.) (13 mL) and THF (13 mL). This afforded 2.4 g (86%), which was used without further purification in the reduction step. The typical procedure for the Reduction was applied and the following reagents were employed: 2-(4-chloro-2-nitrophenoxy)benzoic acid (2.4 g, 0.008 mol), 2 M K₂CO₃ (aq.) (20 mL) and Na₂S₂O₄ (6.72 g, 0.015 mol). This afforded 1.1 g (52%), which was used in the EDC coupling without further purification. The typical procedure Method A for the EDC coupling was applied to form the title compound and the following reagents were employed: 2-(2-amino-4-chlorophenoxy)benzoic acid (500 mg, 1.90 mmol), EDC (540 mg, 2.85 mmol), HOBt (382 mg, 2.85 mmol), DMAP (2.29 mg, 0.019 mmol), TEA (1.22 mL, 8.55 mmol), MeCN (1.8 mL). This afforded 307 mg (66%) of the title compound as a white powder, which was sufficiently pure to be used without further purifications. ¹H NMR (400 MHz, DMSO-d₆): δ 10.61 (1H, br s), 7.76 (1H, dd, J=7.6, 1.6 Hz), 7.64-7.58 (1H, m), 7.38-7.28 (3H, m), 7.20-7.12 (2H, m). ¹³C NMR (100 MHz, DMSO-d₆): δ 166.3, 159.2, 149.8, 135.4, 133.3, 132.2, 130.2, 126.4, 126.0, 125.4, 123.7, 121.6, 121.3.

### Example 13

### 10H-dibenzo[b,f][1,4]thiazepine-11-one (7)

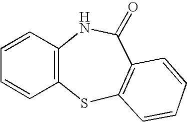

The typical procedure for the Hydrolysis was applied and the following reagents were employed: 2-(2-Nitro-phenylsulfanyl)benzoic acid methyl ester (2.30 g, 7.95 mmol), 2 M LiOH (aq.) (13 mL) and THF (13 mL). This afforded 2.14 (98%), which was used without further purification in the reduction step. The typical procedure for the Reduction was applied and the following reagents were employed: 2-(2-Nitro-phenylsulfanyl)-benzoic acid (2.14 g, 7.84 mmol), 2 M K₂CO₃ (aq.) (20 mL), added Na₂S₂O₄ (6.82 g, 39.0 mol). This afforded 1.2 g (63%), which was used in the EDC coupling without further purification. The typical procedure Method A for the EDC coupling was applied to form the title compound and the following reagents were employed: 2-(2-Amino-phenylsulfanyl)-benzoic acid (500 mg, 2.05 mmol), EDC (587 mg, 3.07 mmol), HOBt (414 mg, 3.07 mmol), DMAP (2.5 mg, 0.02 mmol), TEA (1.28 mL, 9.23 mmol), MeCN (2.0 mL). This afforded 293 mg (63%) of the title compound as a yellow solid, which was sufficiently pure to be used without further purifications. ¹H NMR (400 MHz, DMSO-d₆): δ 10.66 (1H, s), 7.68 (1H, dd, J=7.2, 2.0 Hz), 7.57-7.41 (4H, m), 7.37-7.33 (1H, m), 7.23 (1H, dd, J=8.0, 1.2 Hz), 7.14 (1H, dt, J=15.2, 7.6, 1.2 Hz). ¹³C NMR (100 MHz, DMSO-d₆): δ 169.1, 140.6, 138.5, 137.0, 133.2, 132.7, 132.1, 131.9, 130.5, 129.7, 129.6, 126.1, 123.9.

### Example 14

### 5,10-dihydro-dibenzo[b,f][1,4]diazepine-11-one (8)

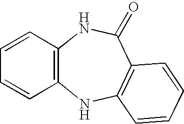

The typical procedure for the Reduction of was applied to form 8 and the following reagents were employed: 2-(2-nitro-phenylamino)-benzoic acid (4.5 g, 19.7 mmol), 2 M K₂CO₃ (aq) (20 mL), Na₂S₂O₄ (17.0 g, 98.7 mmol). This afforded 1.56 g (35%), which was used in the EDC coupling without further purification. The typical procedure Method B for the EDC coupling was applied to form the title compound and the following reagents were employed: 2-(2-amino-phenylamino)-benzoic acid (1.56 g, 6.80 mmol), DCM (30 mL), EDC (1.97 g, 10.3 mmol). This afforded 1.3 g (91%) of the title compound as a yellow powder, which was sufficiently pure to be used without further purifications. ¹H NMR (400 MHz, DMSO-d₆): δ 8.95 (1H, bs), 7.86 (1H, dd, J=1.2 Hz), 7.38-7.34 (1H, m), 7.21 (1H, bs), 7.14-7.12 (1H, m), 7.10-7.05 (2H, m), 7.02-6.94 (3H, m). ¹³C NMR (100 MHz, DMSO-d₆): δ 168.2, 150.7, 140.2, 133.5, 132,7, 130.5, 124.9, 123.6, 123.4, 121.4, 121.2, 120.2, 119.4.

### Example 15

### 4-(2-methoxycarbonyl-phenylsulfanyl)-3-nitro-benzoic acid ethyl ester (9)

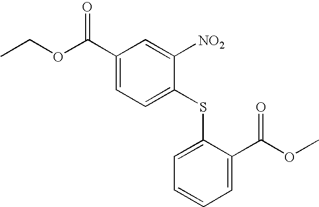

A solution of ethyl 4-flouro-3-nitrobenzoate (2.50 g, 11.7 mmol) and methyl 2-mercaptobenzoate (3.95 g, 23.5 mol) in DMF (20 mL) was added Cs₂CO₃ (7.6 g, 23.5 mol) and the resulting mixture was stirred at 40° C. for 2 hours. The reaction mixture was cooled to room temperature, diluted with EtOAc, washed with water, dried (Na₂SO₃), filtered and evaporated to give crude product. Recrystallization from EtOAc/heptane gave 3.10 g (73%) of the title compound as yellow crystals. ¹H NMR (400 MHz, CDCl₃): δ 8.82 (d, 1H, J=1.9 Hz), 7.94 (m, 2H), 7.62-7.57 (m, 3H), 6.92 (d, 1H, J=8.6 Hz), 4.38 (q, 2H, J=7.2 Hz), 3.78 (s, 3H), 1.38 (t, 3H, J=7.0 Hz); ¹³C NMR (100 MHz, CDCl₃): δ 166.8, 164.6, 145.5, 144.1, 137.6, 136.3, 133.4, 133.0, 131.5, 131.3, 130.5, 129.8, 128.1, 126.9, 61.9, 52.7, 14.5;

### Example 16

### 3-Amino-4-(2-carboxy-phenylsulfanyl)-benzoic acid (1)

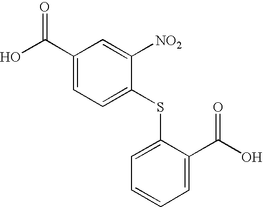

The typical procedure for the Hydrolysis was applied to form the title compound and the following reagents were employed: 4-(2-methoxycarbonyl-phenylsulfanyl)-3-nitro-benzoic acid ethyl ester (3.10 g, 8.60 mmol), 2 M LiOH (aq.) (27 mL) and THF (20 mL). This afforded 2.36 g (86%) of the title compound as yellow crystals which was sufficiently pure to be used in the next step without further purifications. ¹H NMR (400 MHz, CD₃OD): δ 8.71 (d, 1H, J=1.8 Hz), 7.95 (m, 2H), 7.64-7.59 (m, 3H), 7.00 (d, 1H, J=8.6 Hz). ¹³C NMR (100 MHz, CD₃OD): δ 168.3, 166.1, 145.9, 143.3, 137.0, 136.5, 133.2, 132.6, 131.2, 131.1, 130.1, 130.0, 128.6, 126.3

### Example 17

### 3-Amino-4-(2-carboxy-phenylsulfanyl)-benzoic acid (11)

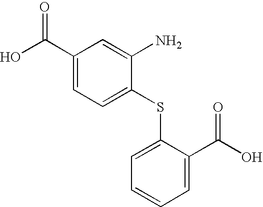

The typical procedure for the Reduction was applied to form the title compound and the following reagents were employed: 3-Amino-4-(2-carboxy-phenylsulfanyl)-benzoic acid (2.36 g, 7.40 mmol), 2 M K₂CO₃ (aq.) (20 mL), Na₂S₂O₄ (8.88 g, 37.0 mmol). This afforded 1.26 g (59%) of the title compound as a white solid, which was sufficiently pure to be used in the next step without further purifications. ¹H NMR (400 MHz, CD₃OD): δ 8.01 (d, 1H, J=7.6 Hz), 7.51 (s, 1H), 7.44 (d, 1H, J=8.0 Hz ), 7.31 (d, 1H, J=8.0 Hz), 7.28 (t, 1H, J=8.0 Hz), 7.16 (t, 1H, J=7.2 Hz), 6.74 (d, 1H, J=8.0 Hz). ¹³C NMR (100 MHz, CD₃OD): 169.8, X, 151.6, 141.6, 138.6, 134.7, 133.5, 132.7, 128.8, 127.2, 125.6, 119.9, 119.6, 117.3.

### Example 18

### 11-Oxo-10,11-dihydro-dibenzo[b,f][1,4]thiazepine-8-carboxylic acid (12)

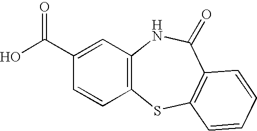

The typical procedure Method C for the EDC coupling was applied to form the title compound and the following reagents were employed: 3-Amino-4-(2-carboxy-phenylsulfanyl)-benzoic acid (400 mg, 1.46 mmol), EDC (807 mg, 4.22 mmol), HOBt (295 mg, 2.19 mmol), DMAP (4.3 mg, 0.03 mmol), TEA (0.90 mL, 6.57 mmol), MeCN (1.5 mL). Purification by recrystallization from EtOH afforded 211 mg (54%) as a off-white solid. ¹H NMR (400 MHz, DMSO-d₆): δ 10.78 (br s, 1H), 7.77 (s, 1H), 7.67 (m, 3H), 7.55-7.42 (m, 3H). ¹³C NMR (100 MHz, DMSO-d₆): δ 168.9, 166.9, 140.3, 138.3, 136.0, 134.5, 133.5, 133.0, 132.9, 132.2, 132.1, 129.9, 126.5, 124.3.

### Example 19

### Typical Procedures for the Synthesis of Imidoyl Chloride

Method A: A mixture of the lactam (1 eq.) in POCl₃ (neat) was heated at 95° C. for 2 hours. The reaction mixture was then cooled to room temperature and excess of POCl₃ was removed at reduced pressure. The resulting residues were dissolved in EtOAc and the organic phase was washed with brine, dried (Na₂SO₃), filtered, and evaporated to give crude product. Purification by flash chromatography.

Method B: A mixture of the lactam (1 eq.), POCl₃ (3 eq.) and N-dimethylaniline (4 eq.) in toluene was heated at 95° C. for 2 hours. The reaction mixture was then cooled to room temperature and excess of POCl₃, N-dimethylaniline and toluene was removed at reduced pressure using an oilpump. The resulting residues were dissolved in dioxane and 2 M Na₂CO₃ (aq) and heated at 80° C. for 1 hour. The reaction mixture was then cooled to room temperature and dioxane was removed at reduced pressure and the resulting aqueous solution was dissolved in EtOAc. The organic phase was washed with water, brine, dried (Na₂SO₃), filtered, and evaporated to give crude product. Purification by flash chromatography.

Method C: A mixture of the lactam (1 eq.) and PCl₅ (5 eq.) in toluene was heated at 110° C. for 2 hours. The reaction mixture was then cooled to room temperature and excess of PCl₅ and toluene was removed at reduced pressure using oilpump to give crude product, which was used without further purification.

### Example 20

### 8,11-dichloro-dibenzo[b,f][1,4]thiazepine (13)

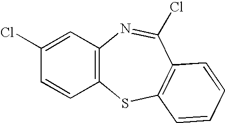

The typical procedure Method A for the Synthesis of Imidoyl chlorides was applied to form the title compound and the following reagents were employed: 8-chloro-10H-dibenzo[b,f][1,4]thiazepin-11-one 5 (500 mg, 1.92 mmol), POCl₃ (15 mL). Purification by flash chromatography (EtOAc/Heptane 1:4) afforded 395 mg (74%) of the title compound as a yellow powder. ¹H NMR (400 MHz, CDCl₃): 7.74 (d, 1H, J=8.0 Hz), 7.45-7.36 (m, 4H), 7.28-7.26 (m, 1H), 7.15-7.12 (m, 1H). ¹³C NMR (100 MHz, CDCl₃): 156.4, 147.1, 138.7, 137.9, 135.4, 133.6, 133.0, 132.2, 130.1, 129.0, 127.4, 126.4, 125.7.

### Example 21

### 8,11-dichlorodibenzo[b,f][1,4]oxazepine (14)

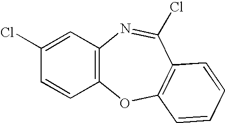

The typical procedure Method A for the Synthesis of Imidoyl chlorides was applied to form the title compound and the following reagents were employed: 8-chloro-10H-dibenzo[b,l][1,4]oxazepin-11-one (490 mg, 2.0 mmol), POCl₃ (15 mL). Purification by flash chromatography (EtOAc/Heptane 1:4) afforded 440 mg (84%) as a white powder. ¹H NMR (400 MHz, Acetone-d₆): δ 7.82 (1H, dd, J=7.6, 1.6 Hz), 7.69 (1H, ddd, J=9.2, 7.6 , 1.6 Hz), 7.41 (1H, ddd, J=8.4, 7.6, 1.2 Hz), 7.36-7.29 (4H, m). ¹³C NMR (100 MHz, Acetone-d₆): δ 160.1, 155.3, 150.5, 139.2, 135.7, 130.9, 130.6, 129.0, 127.3, 127.1, 126.3, 122.9, 121.3.

### Example 22

### 8,11-dichloro-5H-dibenzo[b,e][1,4]diazepine (14)

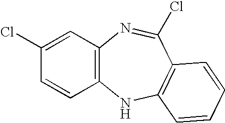

The typical procedure Method B for the Synthesis of Imidoyl chlorides was applied to form the title compound and the following reagents were employed: 8-chloro-11-oxo-10,11-dihydro-5H-dibenzo-1,4-diazepine (2.90 g, 20 mmol), POCl₃ (5.6 mL, 60 mmol), N-dimethylaniline (10.2 mL, 80 mmol), toluene (40 mL) Na₂CO₃ (2 M, 10 ml), dioxane (10 ml). Purification by flash chromatography (EtOAc/Heptane 1:4) afforded 3.76 g (71.5%) of the title compound as a yellow powder. ¹H NMR (400 MHz, CDCl₃): δ 7.59 (1H, dd, J=8.0, 1.6 Hz), 7.31 (1H, dt, J=7.6, 1.6 Hz), 7.15 (1H, d, J=2.4 Hz), 7.04-7.00 (2H, m), 6.63 (1H, dd, J=8.0, 1.2 Hz), 6.58 (1H, d, J=8.4 Hz), 4.95 (1H, bs). ¹³C NMR (100 MHz, CDCl₃): δ 157.2, 152.1, 140.3, 138.3, 134.1, 132.0, 129.8, 128.6, 128.1, 127.1, 123.6, 121.0, 119.8.

A mixture of 8-chloro-11-oxo-10,11-dihydro-5H-dibenzo-1,4-diazepine (2.90 g, 20 mmol), POCl₃ (5.6 mL, 60 mmol) and N-dimethylaniline (10.2 mL, 80 mmol) in toluene (40 ml) was heated at 95° C. for 2 hours. The reaction mixture was then cooled to room temperature and excess of POCl₃, N-dimethylaniline and toluene was removed at reduced pressure using oilpump. The resulting residue was dissolved in dioxane (20 ml) and 2 M Na₂CO₃ (30 ml, 0.06 mol) and heated at 80° C. for 1 hour. The reaction mixture was then cooled to room temperature and dioxane was removed at reduced pressure and the resulting aqueous solution was diluted with EtOAc. The organic phase was washed with water, brine, dried (Na₂SO₄). Filtration, removal of the solvent at reduced pressure gave the crude product. Purification by column chromatography (EtOAc/Heptane 1:4) afforded 3.76 g (72%) of the title compound as a yellow powder.

### Example 23

### 11-chloro-dibenzo[b,f][1,4]oxazepine (15)

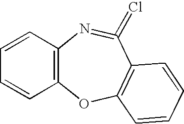

The typical procedure Method A for the Synthesis of Imidoyl chlorides was applied to form the title compound and the following reagents were employed: 10H-dibenzo[b,f][1,4]oxazepine-11-one (500 mg, 2.37 mmol), POCl₃ (15 mL). Purification by flash chromatography (EtOAc/Heptane 1:4) afforded 424 mg (78%) of the title compound as a yellow oil. ¹H NMR (400 MHz, Acetone-d₆): δ 7.81-7.79 (1H, m), 7.69-7.65 (1H, m), 7.40-7.24 (6H, m). ¹¹³C NMR (100 MHz, Acetone-d₆): δ 160.3, 153.5, 151.7, 138.2, 135.4, 130.4, 129.4, 127.9, 127.2, 126.4, 126.0, 121.4, 121.3.

### Example 24

### 11-chloro-dibenzo[b,f][1,4]thiazepine (16)

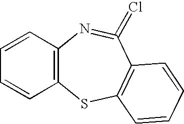

The typical procedure Method A for the Synthesis of Imidoyl chlorides was applied to form the title compound and the following reagents were employed: 10H-dibenzo[b,f][1,4]thiazepine-11-one 7 (594 mg, 2.61 mmol), POCl₃ (15 mL).). Purification by flash chromatography (EtOAc/Heptane 1:4) afforded 419 mg (65%) of the title compound as a yellow solid. ¹H NMR (400 MHz, DMSO-d₆): δ 7.79 (1H, m), 7.60-7.50 (4H, m), 7.43-7.39 (1H, m), 7.29-7.46 (2H, m). ¹³C NMR (100 MHz, DMSO-d₆): δ 154.8, 146.1, 138.6, 137.5, 134.2, 133.4, 132.7, 130.8, 130.5, 130.1, 128.6, 127.6, 126.1.

### Example 25

### 11-Chloro-dihydro-dibenzo[b,f][1,4]thiazepine-8-carbonyl chloride (18)

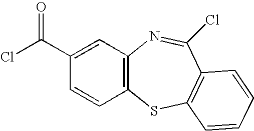

The typical procedure Method C for the Synthesis of Imidoyl chlorides was applied to form the title compound and the following reagents were employed: 11-Oxo-10,11-dihydro-dibenzo[b,f][1,4]thiazepine-8-carboxylic acid (200 mg, 0.74 mmol), PCd₅ (756 mg, 3.68 mmol), toluene (4 mL). This afforded 193 mg (85%) of the title compound as a yellow solid, which was sufficiently pure to be used without further purification. ¹H NMR (400 MHz, CDCl₃): δ 8.01 (d, 1H, J=2.0 Hz), 7.87 (dd, 1H, J=8.4, 2.2 Hz), 7.77 (m, 1H), 7.58 (d, 1H, J=8.2 Hz), 7.47-7.44 (m, 2H), 7.44-7.39 (m, 1H). ¹³C NMR (100 MHz, CDCl₃): δ 167.5, 157.1, 146.7, 137.8, 137.4, 136.3, 134.5, 133.4, 133.3, 132.6, 130.3, 129.5, 129.1, 128.8.

Alternative synthesis of 11-Chloro-dihydro-dibenzo[b,f][1,4]thiazepine-8-carbonyl chloride (13)

A solution of SOCl2 (25 ml), 11-Oxo-10,11-dihydro-dibenzo[b,f][1,4]thiazepine-8-carboxylic acid (1.24 g, 4.6 mmol) and DMF (0.05 ml) in toluene (25 ml) was heated at 90° C. for 6 h. Toluene and excess SOCl₂ were removed at reduced pressure to give the title compound as a yellow solid, which was used in the next step without further purifications.

### Example 26

### General Procedure for Amide Formation

A flame-dried flask was charged under argon with 5 (180 mg; 0.58 mmol) in 4 mL dry DCM and cooled to 0° C. The amine (1.45 mmol) was then slowly added and the reaction was allowed to reach room temperature and stirred for 30 min. The reaction was diluted with DCM and the organic phase was washed with NH₄Cl (aq), brine and dried (Na₂SO₄). Filtration and evaporation at reduced pressure followed by purification by column chromatography (ethyl acetate/heptane 1:1) gave the following compounds (72-88%) as off-white solids.

### Example 27

### (11-chloro-dibenzo[b,f][1,4]thiazepin-8-yl)-[2,4-dimethyl-phenyl)-piperazin-1-yl]-methanone

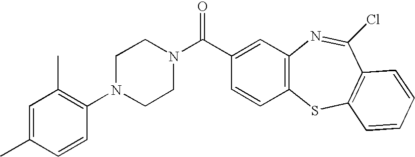

The reaction was performed according to the general procedure, which gave 220 mg (82%) of the title compound. ¹H NMR (400 MHz, CDCl₃) 6 7.75 (m, 1H), 7.51 (d, 1H, J=8.0 Hz), 7.47-7.44 (m, 2H), 7.44-7.39 (m, 1H), 7.31 (d, 1H, J=1.8 Hz), 7.24 (dd, 1H, J=7.8, 1.8 Hz), 7.02 (br s, 1H), 6.98 (br d, 1H, J=8.0 Hz), 6.89 (d, 1H, J=8.0 Hz), 3.88 (br s, 2H), 3.54 (br s, 2H), 2.85 (br s, 4H), 2.28 (s, 6H); ¹³C NMR (100 MHz, CDCl₃) δ 169.0, 156.2, 148.5, 146.4, 138.4, 137.9, 137.6, 133.6, 133.3, 133.1, 132.9, 132.3, 132.1, 130.2, 129.5, 129.1, 127.4, 126.1, 124.3, 119.4, 31.1, 20.9, 17.8; MS (ES⁺, M)=462.

### Example 28

### 11-chloro-dibenzo[b,f][1,4]thiazepin-8-carboxylic acid piperidin-1-ylamide

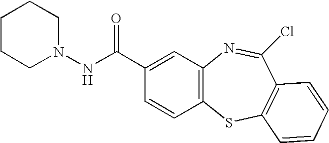

The reaction was performed according to the general procedure, which gave 157 mg (72%) of the title compound. ¹H NMR (400 MHz, CDCl₃) δ 7.74 (m, 1H), 7.59 (dd, 1H, J=8.0, 1.8 Hz), 7.54 (s, 1H), 7.50 (d, 1H, J=8.2 Hz), 7.47-7.43 (m, 2H), 7.43-7.39 (m, 1H), 2.80 (br s, 4H), 1.74 (br s, 4H), 1.44 (br s, 2H); ¹³C NMR (100 MHz, CDCl₃) δ 164.3, 156.6, 146.5, 138.5, 138.1, 135.8, 133.5, 133.4, 132.7, 131.8, 130.4, 129.4, 126.7, 124.2, 57.7, 32.4, 25.8; MS (ES⁺, M+1)=372.

### Example 29

### 4-[(11-chloro-dibenzo[b,f][1,4]thiazepine-8-carbonyl)-amino]-piperidine-1-carboxylic acid ethyl ester

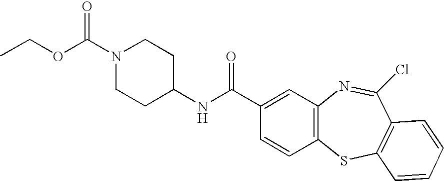

The reaction was performed according to the general procedure, which gave 189 mg (88%) of the title compound. ¹H NMR (400 MHz, CDCl₃) δ 7.74 (m, 1H), 7.61 (dd, 1H, J=8.2, 1.9 Hz), 7.56 (d, 1H, J=1.6 Hz), 7.51 (d, 1H, J=8.2 Hz), 7.47-7.44 (m, 2H), 7.44-7.39 (m, 1H), 6.00 (d, 1H, J=7.6 Hz), 4.12 (m, 5H), 2.94 (t, 2H, J=11.9 Hz), 2.00 (m, 2H), 1.38 (m, 2H), 1.26 (dt, 3H, J=7.2, 1.6 Hz); ¹³C NMR (100 MHz, CDCl₃) δ 165.6, 156.3, 155.7, 146.3, 138.3, 137.8, 136.0, 133.2, 133.2, 132.4, 131.6, 130.2, 129.1, 126.2, 123.9, 61.7, 47.5, 43.0, 32.2, 14.9; MS (ES⁺, M+1)=444.

### Example 30

### Typical Procedure for the Iron-catalyzed Cross-coupling Reaction of Imidoyl Chloride Amide with Alkyl- and Arylmagnesium Halides

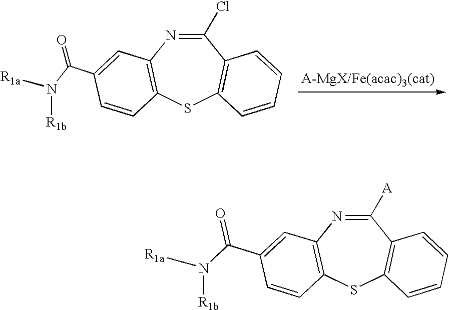

A flame dried 10 mL flask was charged under argon with the imidoyl chloride amide (1 eq.) and Fe(acac)₃ (5 mol %) in dry THF and NMP. Alkyl or arylmagnesium halide (2 eq., 2 M in Et₂O) was slowly added to the red solution, causing an immediate color change to dark brown. The reaction was stirred for 5 min. (at −78° C. rt), then quenched with NH₄Cl (sat., aq.) and diluted with Et₂O. The organic phase was washed with water, brine, dried (Na₂SO₃), filtered, and evaporated to give crude product. Purification by flash chromatography.

### Example 31

### Typical Procedure for the Iron-catalyzed Cross-coupling Reaction of Imidoyl Chloride Amide with Functionalized Arylmagnesium Chloride

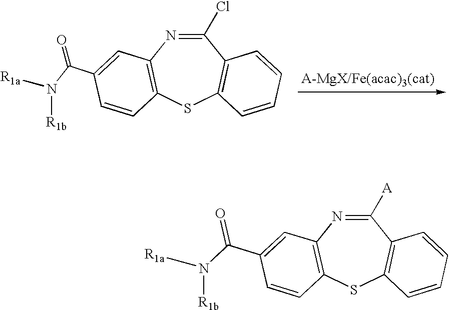

A flame dried 10 mL flask was charged under argon with the imidoyl chloride amide (1 eq.), Fe(acac)₃ (5 mol %) in dry THF and cooled to −40° C. Functionalized arylmagnesium halide (2 eq., 1 M in THF; prepared at −40° C.) was slowly added to the solution, keeping the temperature below −40° C. The reaction was stirred for 5 min. at −40° C., then quenched with NH₄Cl (sat., aq.) and allowed to warm to room temperature. The resulting mixture was diluted with Et₂O and the organic phase was washed with water, brine, dried (Na₂SO₃), filtered, and evaporated to give crude product. Purification by flash chromatography.

### Example 32

### Typical Procedure for the Cross-coupling Reaction of Imidoyl Chloride with Organomanganese Reagents

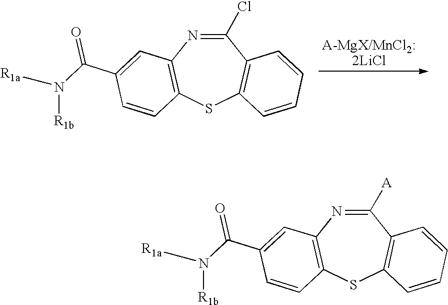

A flame dried 10 mL flask was charged under argon with a solution of MnCl₂:2LiCl (1 eq., 1 M in THF). Alkylmagnesium chloride (2 M in Et₂O) was slowly added and the resulting mixture was stirred for ½ h at rt. Then a solution of the imidoyl chloride amide (1 eq.) in THF was slowly added causing an immediate color change to dark brown. The reaction was stirred for 5 min at room temperature, then quenched with NH₄Cl (sat., aq.) and diluted with Et₂O. The organic phase was washed with water, brine, dried (Na₂SO₃), filtered, and evaporated to give crude product. Yield was measured by ¹H-NMR using anisole as internal standard.

### Example 33

### 11-butyl-dibenzo[b,f][1,4]oxazepine (21)

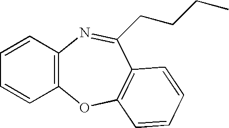

The typical procedure for the Manganese-catalyzed cross-coupling reaction of Imidoyl chloride with alkylmagnesium chloride was applied to form the title compound and the following reagents were employed: 11-chloro-dibenzo[b,f][1,4]oxazepine (47.7 mg, 0.21 mmol), MnCl₂ (2.6 mg, 0.021 mmol), THF (2 mL) and N-methylpyrrolidone (0.2 mL), of nButyl magnesium chloride (2 M in Et₂O, 0.25 mL, 0.42 mmol). ¹H-NMR yield based on anisole as an internal standard showed 21 in >95% yield.

The typical procedure for the cross-coupling reaction of imidoyl chlorides with organomanganese reagent was applied to form the title compound and the following reagents were employed: 11-chloro-dibenzo[b,f][1,4]oxazepine (47.7 mg, 0.21 mmol), Fe(acac)₃ (3.53 mg, 0.001 mmol), MnCl₂:2LiCl (1 M, 0.27 mL, 0.27 mmol), THF (2 mL) and N-methylpyrrolidone (0.2 mL), nButyl magnesium chloride (2 M in Et₂O, 0.25 mL, 0.42 mmol). ¹H-NMR yield based on anisole as an internal standard showed 33 in >95% yield.

The typical procedure for the Iron-catalyzed cross-coupling reaction of imidoyl chlorides with alkylmagnesium was applied to form the title compound and the following reagents were employed: 11-chloro-dibenzo[b,f][1,4]oxazepine (46 mg, 0.20 mmol), Fe(acac)₃ (3.53 mg, 0.001 mmol), THF (2 mL) and N-methylpyrrolidone (0.2 mL), nButyl magnesium chloride (1.7 M in Et₂O, 0.24 mL, 0.40 mmol). Purification by flash chromatography (ethyl acetate/heptane 1:4) afforded 47.7 mg (95%) of the title compound as an oil. ¹H NMR (400 MHz, CDCl₃): δ 7.44-7.41 (2H, m), 7.30-7.26 (1H, m), 7.22-7.18 (2H, m), 7.16-7.13 (3H, m), 2.93 (2H, t, J=7.2 Hz), 1.71 (2H, quintet, J=7.6 Hz), 1.46 (2H, sextet, J=7.2 Hz), 0.99 (3H, t, J=7.2 Hz). ¹³C NMR (100 MHz, CDCl₃): δ 171.2, 161.7, 152.8, 141.0, 132.7, 128.7, 128.4, 127.9, 127.2, 125.7, 125.3, 121.1, 120.8, 40.2, 29.9, 22.7, 14.2.

The reaction was performed according to the typical procedure using 11-chloro-dibenzo[b,f][1,4]oxazepine (46 mg, 0.20 mmol) except that FeCl₃ (2 mg, 0.01 mmol) was used as catalyst in this reaction. ¹H-NMR yield based on anisole as an internal standard showed the title compound in 95% yield.

### Example 34

### 11-cyclohexyl-dibenzo[b,f][1,4]oxazepine (22)

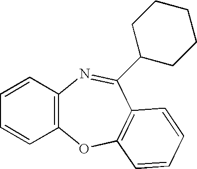

The typical procedure for the Iron-catalyzed cross-coupling reaction of imidoyl chlorides with alkylmagnesium was applied to form the title compound and the following reagents were employed: 11-chloro-dibenzo[b,f][1,4]oxazepine (46 mg, 0.20 mmol), Fe(acac)₃ (3.53 mg, 0.001 mmol), THF (2 mL) and N-methylpyrrolidone (0.2 mL), of cyclohexyl magnesium chloride (2 M in Et₂O, 0.20 mL, 0.40 mmol). Purification by flash chromatography (ethyl acetate/heptane 1:4) afforded 51.5 mg (93%) of the title compound as an oil. ¹H NMR (400 MHz, CDCl₃): δ 7.44-7.37 (2H, m), 7.28-7.25 (1H, m), 7.20-7.09 (5H, m), 2.91 (1H, tt, J=14.8, 3.2 Hz), 2.00-1.97 (2H, m), 1.89-1.85 (2H, m), 1.75-1.71 (1H, m), 1.67-1.55 (2H, m), 1.45-1.24 (3H, m). ¹³C NMR (100 MHz, CDCl₃): δ 174.1, 162.0, 152.7, 141.2, 132.3, 128.9, 127.9, 126.9, 125.6, 125.2, 120.9, 120.6, 47.0, 31.6, 26.6, 26.4.

### Example 35

### 11-tertbutyl-dibenzo[b,f][1,4]oxazepine (23)

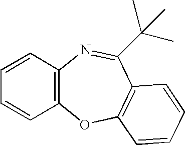

The typical procedure for the iron-catalyzed cross-coupling reaction of imidoyl chlorides with alkylmagnesium was applied to form the title compound and the following reagents were employed: 11-chloro-dibenzo[b,f][1,4]oxazepine (46 mg, 0.20 mmol), Fe(acac)₃ (3.53 mg, 0.001 mmol), dry THF (2 mL) and N-methylpyrrolidone (0.2 mL), tbutyl magnesium chloride (M in Et₂O, mL, mmol). Purification by flash chromatography (ethyl acetate/heptane 1:4) afforded 14 mg (27%) of the title compound as an oil. ¹H NMR (400 MHz, CDCl₃): δ 7.54-7.52 (1H, m), 7.73-7.33 (1H, m), 7.22-7.17 (2H, m), 7.15-7.05 (4H, m), 1.43 (9H, s).

### Example 36

### 11-methyl-dibenzo[b,f][1,4]oxazepine (24)

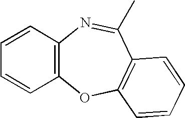

The typical procedure for the iron-catalyzed cross-coupling reaction of imidoyl chlorides with alkylmagnesium was applied to form the title compound and the following reagents were employed: 11-chloro-dibenzo[b,f][1,4]oxazepine (46 mg, 0.20 mmol), Fe(acac)₃ (3.53 mg, 0.001 mmol), dry THF (2 mL) and N-methylpyrrolidone (0.2 mL), trimethylsilyl magnesium chloride (2 M in Et₂O, 0.20 mL, 0.40 mmol). Purification by flash chromatography (ethyl acetate/heptane 1:4) afforded 30.1 mg (72%) of the title compound as an oil.

The typical procedure for the iron-catalyzed cross-coupling reaction of imidoyl chlorides with alkylmagnesium was applied to form the title compound and the following reagents were employed: 11-chloro-dibenzo[b,f][1,4]oxazepine (46 mg, 0.2 mmol), Fe(acac)₃ (3.53 mg, 0.001 mmol), dry THF (2 mL) and N-methylpyrrolidone (0.2 mL), methyl magnesium chloride (1 M in THF, 0.40 mL, 0.40 mmol). ¹H-NMR yield based on toluene as an internal standard showed the title compound in 17% yield. ¹H NMR (400 MHz, CDCl₃): δ 7.45-7.40 (2H, m), 7.29-7.26 (1H, m), 7.22-7.14 (5H, m), 2.65 (3H, s). ¹³C NMR (100 MHz, CDCl₃): δ 167.5, 161.1, 152.7, 140.9, 132.9, 129.3, 128.7, 127.9, 127.4, 125.7, 125.2, 121.0, 120.8, 27.8.

### Example 37

### 11-(2-[1,3]dioxane-2-yl-ethyl)-dibenzo[b,f][1,4]oxazepine (25)

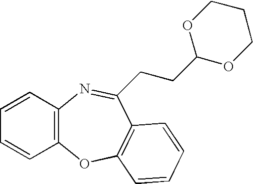

The typical procedure for the iron-catalyzed cross-coupling reaction of imidoyl chlorides with alkylmagnesium halide was applied to form the title compound and the following reagents were employed: 11-chloro-dibenzo[b,f][1,4]oxazepine (46 mg, 0.20 mmol), Fe(acac)₃ (3.53 mg, 0.001 mmol), THF (2 mL) and N-methylpyrrolidone (0.2 mL), 1,3-dioxane-2-yl-ethyl magnesium bromide (0.5 M in Et₂O, 0.80 mL, 0.40 mmol). Purification by flash chromatography (ethyl acetate/heptane 1:4) afforded 58.7 mg (95%) of the title compound as an oil. ¹H NMR (400 MHz, CDCl₃): δ 7.47 (1H, dd, J=7.6, 1.2 Hz), 7.40 (1H, dt, J=8.0, 1.6 Hz), 7.28-7.25 (1H, m), 7.20-7.12 (5H, m), 4.69 (1H, t, J=5.2 Hz), 4.10 (2H, dd, J=11.6, 4.8 Hz), 3.75 (2H, dt, J=12.0, 1,6 Hz), 3.03 (2H, t, J=4.8 Hz), 2.13-2.02 (3H, m), 1.35-1.30 (1H, m). ¹³C NMR (100 MHz, CDCl₃): δ 169.76, 161.5, 152.7, 141.0, 132.7, 128.9, 128.4, 128.0, 127.2, 125.6, 125.3, 120.9, 120.7, 101.6, 67.1, 34.1, 32.6, 26.0.

### Example 38

### 11-phenyl-dibenzo[b,f][1,4]oxazepine (26)

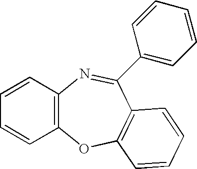

The typical procedure for the iron-catalyzed cross-coupling reaction of imidoyl chlorides with phenylmagnesium halide was applied to form the title compound and the following reagents were employed: 11-chloro-dibenzo[b,f][1,4]oxazepine (46 mg, 0.20 mmol), Fe(acac)₃ (3.53 mg, 0.001 mmol), THF (2 mL) and N-methylpyrrolidone (0.2 mL), phenyl magnesium bromide (2 M in Et₂O, mL, mmol). Purification by flash chromatography (ethyl acetate/heptane 1:4) afforded 29.8 mg (55%) of the title compound as an oil. ¹H NMR (400 MHz, CDCl₃): δ 7.84-7.81 (2H, m), 7.51-7.42 (5H, m), 7.28-7.27 (1H, m), 7.23-7.13 (5H, m). ¹³C NMR (100 MHz, CDCl₃): δ 167.2, 162.2, 152.6, 141.0, 140.3, 133.1, 131.4, 130.5, 129.8, 128.4, 128.3, 127.7, 127.6, 125.7, 124.6, 121.1, 120.8.

The reaction was performed according to the typical procedure using 11-chloro-dibenzo[b,f][1,4]oxazepine (49 mg, 0.20 mmol) and phenylmagnesium chloride (0.60 ml, 1.2 mmol) except that the solvent (Et₂O) was used and the reaction time (30 min) was extended. The yield of the title compound was determined to be 41% according to ¹H-NMR analysis using toluene as an internal standard.

### Example 39

### 11-butyl-dibenzo[b,f][1,4]thiazepine (27)

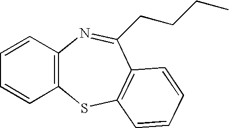

The typical procedure for the iron-catalyzed cross-coupling reaction of imidoyl chlorides with alkylmagnesium halide was applied to form the title compound and the following reagents were employed: 11-chloro-dibenzo[b,f][1,4]thiazepine (49 mg, 0.20 mmol), Fe(acac)₃ (3.53 mg, 0.001 mmol), THF (2 mL) and N-methylpyrrolidone (0.2 mL), nButyl magnesium chloride (2 M in Et₂O, 0.20 mL, 0.40 mmol). Purification by flash chromatography (ethyl acetate/heptane 1:4) afforded 49.6 mg, (93%) of the title compound as an oil. ¹H NMR (400 MHz, CDCl3): δ 7.47-7.44 (1H, m), 7.42-7.37 (2H, m), 7.32-7.30 (2H, m), 7.27-7.23 (1H, m), 7.19-7.17 (1H, m), 7.05-7.01 (1H, m), 3.02-2.83 (2H, m), 1.70-1.61 (2H, m), 1.53-1.43 (2H, m), 0.93 (3H, t, J=7.6 Hz). ¹³C NMR (100 MHz, CDCl3): δ 173.6, 148.9, 140.7, 139.1, 132.5, 132.0, 130.6, 129.2, 129.0, 128.5, 127.8, 125.5, 125.4, 42.3, 29.7, 22.7, 14.1.

### Example 40

### 11-cyclohexyl-dibenzo[b,f][1,4]thiazepine (28)

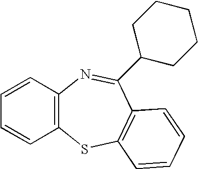

The typical procedure for the iron-catalyzed cross-coupling reaction of imidoyl chlorides with alkylmagnesium halide was applied to form the title compound and the following reagents were employed: 11-chloro-dibenzo[b,f][1,4]thiazepine (49 mg, 0.20 mmol), Fe(acac)₃ (3.53 mg, 0.001 mmol), THF (2 mL) and N-methylpyrrolidone (0.2 mL), cyclohexyl magnesium chloride (2 M in Et₂O, 0.20 mL, 0.40 mmol). Purification by flash chromatography (ethyl acetate/heptane 1:4) afforded 51.9 mg (89%) of the title compound as an oil. ¹H NMR (400 MHz, CDCl₃): δ 7.45-7.43 (1H, m), 7.04-7.36 (2H, m), 7.33-7.21 (4H, m), 7.16 (1H, d, J=8.0, 1.6 Hz), 7.02-6.98 (1H, m), 2.86 (1H, tt, J=11.2, 3.2 Hz), 2.19-2.15 (1H, m), 1.96-1.92 (1H, m), 1.86-1.70 (4H, m), 1.45-1.26 (4H, m).¹³C NMR (100 MHz, CDCl₃): δ 176.7, 149.0, 141.2, 139.4, 132.4, 131.8, 130.2, 129.1, 128.9, 128.5, 127.4, 125.4, 125.1, 49.1, 32.6, 30.2, 27.0, 26.4, 26.2.

### Example 41

### 11-(2-[1,3]dioxane-2-yl-ethyl)-dibenzo[b,f][1,4]thiazepine (29)

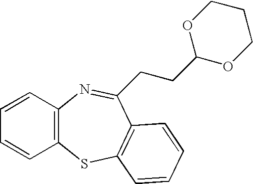

The typical procedure for the iron-catalyzed cross-coupling reaction of imidoyl chlorides with alkylmagnesium halide was applied to form the title compound and the following reagents were employed: 11-chloro-dibenzo[b,f][1,4]thiazepine (49 mg, 0.20 mmol), Fe(acac)₃ (3.53 mg, 0.001 mmol), THF (2 mL) and N-methylpyrrolidone (0.2 mL), of 1,3-dioxane-2-yl-ethyl magnesium bromide (0.5 M in Et₂O, 0.40 mL, mmol). Purification by flash chromatography (ethyl acetate/heptane 1:4) afforded 56 mg (86%) as an oil. ¹H NMR (400 MHz, CDCl₃): 7.45-7.39 (3H, m), 7.31-7.22 (3H, m), 7.18-7.15 (1H, m), 7.05-7.00 (1H, m), 4.74 (1H, t, J=5.2 Hz), 4.12-4.07 (2H, m), 3.80-3.71 (2H, m), 3.07-3.00 (2H, m), 2.12-2.01 (3H, m), 1.34-1.30 (1H, m). ¹³C NMR (100 MHz, CDCl₃): δ 172.3, 148.9, 140.6, 139.1, 132.5, 132.0, 130.7, 129.2, 128.9, 128.6, 127.9, 125.5 (2C), 101.5, 67.1, 36.2, 32.4, 26.1.

### Example 42

### 8-chloro-11-(1-methyl-piperidine-4-yl)-5H-dibenzo[b,e][1,4]diazepine (33)

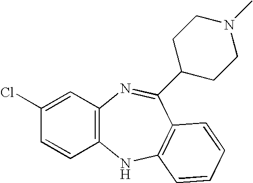

The typical procedure for the Iron catalyzed cross-coupling reaction of imidoyl chlorides with alkylmagnesium halide was applied to form the title compound and the following reagents were employed: 8,11-dichloro-5H-dibenzo[b,e]-1,4-diazepine (53 mg, 0.2 mmol), Fe(acac)₃ (3.53 mg, 0.001 mmol), THF (2 mL) and N-methylpyrrolidone (0.2 mL), 4-methylpiperidine magnesium chloride (1 M in THF, 0.40 mL, 0.40 mmol). Purification by flash chromatography (ethyl acetate/heptane/MeOH/Et₃N 2:1:5%:1%) afforded 53 mg (82%) of the title compound as an oil. ¹H NMR (400 MHz, CD₃OD): δ 7.32 (1H, dd, J=7.6, 1.2 Hz), 7.24-7.22 (1H, m), 7.00-6.96 (2H, m), 6.93 (1H, dd, J=8.4, 2.4 Hz), 6.83 (1H, dd, J=8.0, 1.2 Hz), 6.75 (1H, d, J=8.4 Hz), 6.64 (1H, bs), 2.96-2.92 (2H, m), 2.87-2.81 (1H, m), 2.28 (3H, s), 2.18-2.11 (2H, m), 1.91-1.83 (4H, m). ¹³C NMR (100 MHz, CD₃OD): δ 176.7, 155.6, 143.0, 142.2, 131.6, 128.3, 128.3, 128.1, 127.0, 125.7, 122.8, 120.6, 119.5, 55.3, 45.1, 44.2, 30.0.

### Example 43

### 8-chloro-11-(1-methyl-piperidine-4-yl)-5H-dibenzo[b,f][1,4]oxazepine (34)

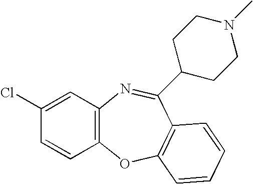

The typical procedure for the Iron catalyzed cross-coupling reaction of imidoyl chlorides with alkylmagnesium halide was applied to form the title compound and the following reagents were employed: 8,11-dichlorodibenzo[b,f][1,4]oxazepine (53 mg, 0.2 mmol), Fe(acac)₃ (3.53 mg, 0.001 mmol), THF (2 mL) and N-methylpyrrolidone (0.2 mL), 4-methylpiperidine magnesium chloride (1 M in THF, 0.40 mL, 0.40 mmol). Purification by flash chromatography (ethyl acetate/heptane/MeOH/Et₃N 2:1:5%:1%) afforded 46 mg (71%) of the title compound as an oil. ¹H NMR (400 MHz, CD₃OD): δ 7.54 (1H, dd, J=8.0, 1.6 Hz), 7.50-7.45 (1H, m), 7.26 (1H, dt, J=7.6, 1.2 Hz), 7.21-7.18 (2H, m), 7.10 (2H, d, J=1.2 Hz), 3.04 (1H, m), 2.95 (2H, m), 2.29 (3H, s), 2.18 (2H, m), 1.92-1.87 (4H, m). ¹³C NMR (100 MHz, CD₃OD): δ 174.1, 161.8, 151.4, 141.9, 133.0, 130.3, 128.0, 127.7, 127.0, 126.7, 125.6, 121.6, 120.6, 55.2, 45.1, 43.4, 30.0.

### Example 44

### 8-chloro-11-(1-methyl-piperidine-4-yl)-5H-dibenzo[b,f][1,4]thiazepine (35)

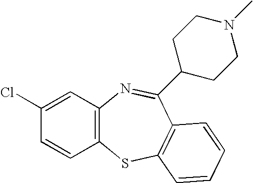

The typical procedure for the Iron catalyzed cross-coupling reaction of imidoyl chlorides with alkylmagnesium halide was applied to form the title compound and the following reagents were employed: 8,11-dichloro-dibenzo[b,f][1,4]thiazepine (56 mg, 0.2 mmol), Fe(acac)₃ (3.53 mg, 0.001 mmol), THF (2 mL) and N-methylpyrrolidone (0.2 mL), 4-methylpiperidine magnesium chloride (1 M in THF, 0.40 mL, 0.40 mmol). Purification by flash chromatography (ethyl acetate/heptane/MeOH/Et₃N 2:1:5%: 1%) afforded 59 mg (86%) of the title compound as an oil. ¹H NMR (400 MHz, CD₃OD): δ 7.49-7.36 (4H, m), 7.43 (1H, d, J=8.4 Hz), 7.10 (1H, d, J=2.4 Hz), 7.01 (1H, dd, J=8.4, 2.0 Hz), 3.04-2.96 (2H, m), 2.86-2.83 (1H, m), 2.28 (3H, s), 2.26-2.19 (1H, m), 2.14-2.06 (3H, m), 1.71-1.57 (2H, m). ¹³C NMR (100 MHz, CD₃OD): δ 176.6, 149.8, 140.5, 138.4, 134.6, 133.1, 131.5, 130.8, 129.0, 127.6, 127.4, 125.0, 124.5, 55.3, 45.1, 31.0, 28.3.

### Example 45

### Typical Procedure Iron-catalyzed Cross-coupling Reaction of Imidoyl Chloride with Functionalized Arylmagnesium Halides

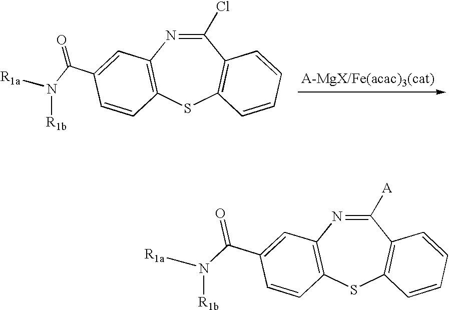

A flame dried 10 ml flask was charged under argon with the imidoyl chloride (47 mg, 0.20 mmol), Fe(acac)₃ (4 mg, 0.001 mmol) in dry THF (2 ml) and cooled to −40° C. 4-methyl benzoate magnesium chloride (1.60 ml, 1.60 mmol) was then slowly added to the solution maintaining the temperature below −40° C. The reaction was stirred for 30 min. at −40° C., then quenched with saturated aqueous NH₄Cl and allowed to warm up to room temperature. The organic phase was washed with water, brine and dried (Na₂SO₄). Filtration and removal of the solvent at reduced pressure gave the crude product crude product.

### Example 46

### 4-dibenzo[b,f][1,4]oxapin-11-yl-benzoic acid methyl ester (36)

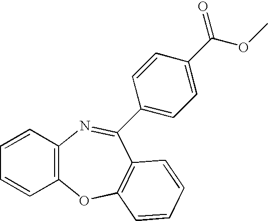

The typical procedure for the iron-catalyzed cross-coupling reaction of imidoyl chlorides with functionalized arylmagnesium halide was applied to form the title compound and the following reagents were employed: 11-chloro-dibenzo[b,f][1,4]oxazepine (47 mg, 0.20 mmol), Fe(acac)₃ (3.53 mg, 0.001 mmol), THF (2 mL), NMP (0.2 mL), 4-methyl benzoate magnesium chloride (1M in THF, 0.80 mL, 0.80 mmol). Purification by Prep TLC (ethyl acetate/heptane) gave 19.7 mg (30%) of the title compound as a yellow solid. ¹H NMR (400 MHz, CDCl₃): δ 8.11 (br d, 2H, J=8.2 Hz), 7.90 (br d, 2H, J=8.0 Hz), 7.52 (m, 1H), 7.44 (m, 1H), 7.28 (d, 1H, J=8.2 Hz), 7.22 (m, 3H), 7.14 (m, 2H), 3.96 (s, 3H); ¹³C NMR (100 MHz, CDCl₃): δ 166.9, 166.3, 162.3, 152.5, 144.4, 140.8, 133.5, 131.8, 131.1, 129.9, 129.6, 128.6, 128.2, 127.3, 125.9, 124.8, 121.3, 121.0, 52.5.

### Example 47

### 4-dibenzo[b,f][1,4]oxaipin-11-yl-benzonitrile (37)

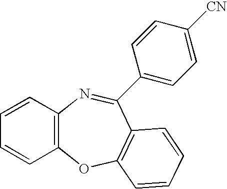

The typical procedure for the iron-catalyzed cross-coupling reaction of imidoyl chlorides with functionalized arylmagnesium halide was applied to form the title compound and the following reagents were employed: 11-chloro-dibenzo[b,f][1,4]oxazepine (246.4 mg, 1.07 mmol), Fe(acac)₃ (18.9 mg, 0.05 mmol), THF (8 mL), 4-benzonitrile magnesium chloride (1M in THF, 4.27 mL, 4.27 mmol). Purification by flash chromatography (tBuMeO:heptane 1:4) and gave 104.5 mg (33%) of the title compound as a yellow oil. ¹H NMR (400 MHz, CDCl₃): δ 7.97-7.93 (2H, m), 7.76-7.73 (2H, m), 7.56-7.51 (1H, m), 7.46-7.42 (1H, m), 7.32-7.28 (1H, m), 7.25-7.16 (4H, m), 7.12-7.08 (1H, m). ¹³C NMR (100 MHz, CDCl₃): δ 165.3, 162.4, 152.4, 144.3, 140.6, 133.7, 132.2, 130.8, 130.4, 128.6, 126.8, 126.0, 125.0, 121.6, 121.1, 118.7, 114.0.

### Example 48

### 11-oxo-10,11-dibenzo[b,f][1,4]thiazepine-8-carboxylic acid methyl ester (38)

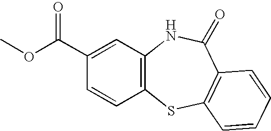

A solution of 11-Oxo-10,11-dihydro-dibenzo[b,f][1,4] thiazepine-8-carboxylic acid (715 mg, 2.61 mmol) and Na₂CO₃ (1.39 g, 13.05 mmol) in DMF (20 mL) was stirred at room temperature for 0.5 hour. Then CH₃I (0.81 mL, 13.05 mmol) was added and the two phase mixture was stirred for another 0.5 hour at room temperature. DMF was then removed at reduce pressure using oil pump and the resulting residue was dissolved in EtOAc. The organic phase was washed with NaHCO₃ (aq. sat.), brine, dried (Na₂SO₃), filtered and evaporated to give crude product. Purification by recrystallization from Toluene afforded 640 mg (86%) of the title compound as a white solid. ¹H NMR (400 MHz, CDCl₃): δ 9.23 (1H, bs), 7.88-7.86 (2H, m), 7.79-7.77 (1H, m), 7.64 (1H, J=8.0 Hz), 7.52-7.50 (1H, m), 7.45-7.38 (2H, m), 3.91 (3H, s). ¹³C NMR (100 MHz, CDCl₃): δ 169.4, 165.9, 139.6, 136.8, 136.4, 135.8, 133.3, 132.7, 132.3, 132.2, 131.8, 129.3, 127.0, 123.7, 52.7.

### Example 49

### 11-chloro-dibenzo[b,f][1,4]thiazepine-8-carboxylic acid methyl ester (39)

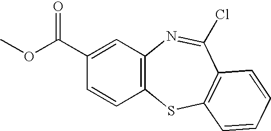

The typical procedure Method C for the Synthesis of Imidoyl chlorides was applied to form the title compound and the following reagents were employed: 11-oxo-10,11-dibenzo[b,f][1,4]thiazepine-8-carboxylic acid methyl ester (540 mg, 1.89 mmol), PCl₅ (1.97 g, 9.47 mmol), toluene (15 mL). Purification by flash chromatography (ethyl acetate/heptane 1:4) afforded 410 mg (71%) of the title compound as a yellow solid. ¹H NMR (400 MHz, CDCl₃): δ 7.86 (1H, dd, J=2.0, 0.4 Hz), 7.75 (1H, dd, J=8.0, 1.6 Hz), 7.69-7.67 (1H, m), 7.45 (1H, dd, J=8.4, 0.4 Hz), 7.40-7.32 (3H, m), 3.82 (3H, s). ¹³C NMR (100 MHz, CDCl₃): δ 166.2, 156.1, 146.3, 138.1, 137.9, 133.2, 133.1, 132.9, 132.4, 131.7, 130.2, 129.2, 128.1, 127.1, 52.6.

### Example 50

### 11-butyl-dibenzo[b,f][1,4]thiazepine-8-carboxylic acid methyl ester (40)

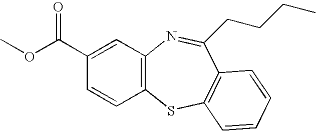

The typical procedure for the Iron-catalyzed cross-coupling reaction of imidoyl chlorides with alkylmagnesium was applied to form the title compound and the following reagents were employed: 11-chloro-dibenzo[b,f][1,4]thiazepine-8-carboxylic acid methyl ester (151.5 mg, 0.50 mmol), Fe(acac)₃ (8.85 mg, 0.05 mmol), THF (4 mL) and N-methylpyrrolidone (0.4 mL), nButyl magnesium chloride (2 M in Et₂O, 0.50 mL, 1.0 mmol). Purification by flash chromatography (ethyl acetate/heptane 1:5) afforded 144 mg (89%) of the title compound as a yellow solid. ¹H NMR (400 MHz, CDCl₃): δ 7.84 (1H, d, J=1.6 Hz), 7.68 (1H, dd, J=8.0, 1.6 Hz), 7.74-7.43 (2H, m), 7.40-7.31 (3H, m), 3.87 (3H, s), 3.02-2.85 (2H, m), 1.74-1.58 (2H, m), 1.55-1.41 (2H, m), 0.93 (3H, t, J=7.2 Hz). ¹³C NMR (100 MHz, CDCl₃): δ 174.5, 166.7, 148.8, 139.7, 139.0, 134.4, 132.5, 132.3, 131.1, 130.9, 128.9, 127.9, 126.6, 126.1, 52.4, 42.2, 29.5, 22.7, 14.2.

### Example 51

### 11-chloro-dibenzo[b,f][1,4]thiazepine-8-carboxylic acid methoxy-methyl-amide (41)

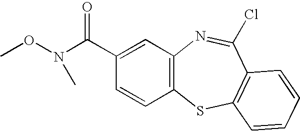

A flame dried 10 mL flask was charged under argon with 8,11-dichloro-dibenzo[b,f][1,4]thiazepine (622 mg, 2.00 mmol) in dry DCM (4 mL) and was then slowly added to a solution of N,O-dimethylhydroxylamine hydrochloride in dry DCM (6 mL) and TEA (4 eq.). The resulting reaction mixture was stirred at rt for 0.5 hour and was diluted with DCM. The organic phase was washed with water, brine, dried (Na₂SO₃), filtered and evaporated to give crude product. Purification by flash chromatography (ethyl acetate/heptane 1:) afforded 518 mg (78%) of the title compound as a yellow oil. ¹H NMR (400 MHz, CDCl₃): δ 7.76-7.73 (1H, m), 7.57-7.56 (1H, m), 7.50-7.83 (5H, m), 3.54 (3H, s), 3.33 (3H, s). ¹³C NMR (400 MHz, CDCl₃): δ 168.5, 156.0, 146.0, 138.4, 137.9, 135.7, 133.1, 132.6, 132.3, 130.4, 130.2, 129.1, 127.2, 125.7, 61.5, 33.8.

### Example 52

### 11-butyl-dibenzo[b,f][1,4]thiazepine-8-carboxylic acid methoxy-methyl-amide (42)

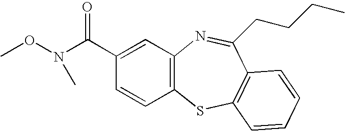

The typical procedure for the Iron-catalyzed cross-coupling reaction of Imidoyl chlorides with alkylmagnesium was applied to form the title compound and the following reagents were employed: 11-chloro-dibenzo[b,f][1,4]thiazepine-8-carboxylic acid methoxy-methyl-amide (61.5 mg, 0.19 mmol), Fe(acac)₃ (3.53 mg, 0.001 mmol), THF (2 mL) and N-methylpyrrolidone (0.20 mL), nButyl magnesium chloride (2 M in Et₂O, 0.11 mL, 0.23 mmol). Purification by flash chromatography (ethyl acetate/heptane 1:1) afforded 47 mg (70%) of the title compound as a yellow oil. ¹H NMR (400 MHz, CDCl₃): δ 7.45-7.42 (3H, m), 7.39-7.29 (4H, m), 3.54 (3H,s), 3.32 (3H,s), 3.01-2.82 (2H, m), 1.69-1.59 (2H, m), 1.51-1.41 (2H, m), 0.92 (3H, t, J=7.2 Hz). ¹³C NMR (100 MHz, CDCl₃): δ 174.4, 169.2, 148.7, 140.0, 139.0, 135.2, 132.2, 132.1, 131.6, 130.8, 128.7, 127.9, 125.1, 124.9, 61.4, 42.2, 34.1, 29.6, 22.7, 14.1.

### Example 53

### (11-butyl-dibenzo[b,f][1,4]thiazepine-8-yl)-cyclohexyl-methanone (43)

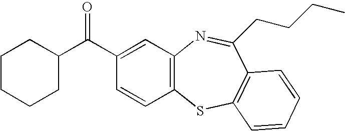

A flame dried 10 mL flask was charged under argon with 11-butyl-dibenzo[b,f][1,4]thiazepine-8-carboxylic acid methoxy-methyl-amide (29 mg, 0.08 mmol) in dry THF (2 mL) and cyclohexyl magnesium chloride (2 M in Et₂O, 0.12 mL, 0.24 mmol) was then added. The resulting reaction mixture was stirred at room temperature for 1 hour and was then diluted with ether. The organic phase was washed with water, brine, dried (Na₂SO₃), filtered and evaporated to give crude product. Purification by Prep. TLC (ethyl acetate/heptane 1:10) afforded 5 mg (17%) of the title compound as a colorless oil. ¹H NMR (400 MHz, CDCl₃): δ 7.70 (1H, d, J=2 Hz), 7.60 (1H, dd, J=8.0, 2.0 Hz), 7.49-7.44 (2H, m), 7.41-7.33 (3H, m), 3.19 (1H, tt, J=11.2, 3.2 Hz), 3.04-2.97 (1H, m), 2.92-2.84 (1H, m), 1.83-1.79 (3H, m), 1.72-1.62 (3H, m), 1.51-1.21 (8H, m), 0.93 (3H, t, J=7.6 Hz). ¹³C NMR (100 MHz, CDCl₃): δ 203.4, 174.7, 148.9, 139.8, 138.9, 137.3, 134.2, 132.8, 132.3, 130.9, 128.9, 127.9, 125.3, 124.9, 45.8, 42.3, 29.6, 29.5, 26.1, 26.0, 22.7, 14.2.

### Example 54

### 1-(11-chloro-dibenzo[b,f][1,4]thiazepine-8-yl)-pentan-1-one (44)

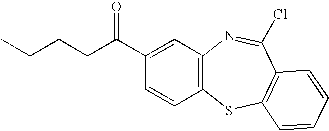

A flame dried 10 mL flask was charged under argon with 11-chloro-dibenzo[b,f][1,4]thiazepine-8-carboxylic acid methoxy-methyl-amide (34 mg, 0.10 mmol) in dry THF (2 mL) and nButyl magnesium chloride (2 M in Et₂O, 0.10 mL, 0.2 mmol) was then added. The resulting reaction mixture was stirred at room temperature for 1 hour and was then diluted with ether. The organic phase was washed with water, brine, dried (Na₂SO₃), filtered and evaporated to give crude product. Purification by flash chromatography (ethyl acetate/heptane 1:5) afforded 26.0 mg (81%) of the title compound as a yellow oil. ¹H NMR (400 MHz, CDCl₃): δ 7.82 (1H, d, J=1.6 Hz), 7.77-7.74 (2H, m), 7.53 (1H, d, J=8.4 Hz), 7.47-7.39 (3H, m), 2.90 (2H, t, J=7.2 Hz), 1.68 (2H, quintet, J=7.2 Hz), 1.37 (2H, sextet, J=7.2 Hz), 0.93 (3H, t, J=7.2 Hz). ¹³C NMR (100 MHz, CDCl₃): δ 199.5, 156.2, 146.4, 138.3, 138.1, 137.8, 133.2, 133.1(2), 132,5, 1302, 129.2, 126.6, 125.8, 38.7, 26.5, 22.6, 14.1.

### Example 55

### 1-(11-cyclohexyl-dibenzo[b,f][1,4]thiazepine-8-yl)-pentan-1-one (45)

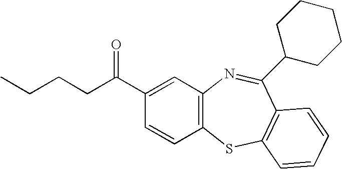

The typical procedure for the Iron-catalyzed cross-coupling reaction of Imidoyl chlorides with alkylmagnesium was applied to form the title compound and the following reagents were employed: 1-(11-chloro-dibenzo[b,f][1,4]thiazepine-8-yl)-pentan-1-one (26.0 mg, 0.08 mmol), Fe(acac)₃ (1.41 mg, 0.004 mmol), THF (2 mL) and N-methylpyrrolidone (0.20 mL), cyclohexyl magnesium chloride (2 M in Et₂O, 0.08 mL, 0.16 mmol). Purification by Prep. TLC (ethyl acetate/heptane 1:10) afforded 17.2 mg (57%) of the title compound as an colorless oil. ¹H NMR (400 MHz, CDCl₃): δ 7.71 (1H, d, J=1.6 Hz), 7.59 (1H, dd, J=8.0, 2.0 Hz), 7.48-7.43 (2H, m), 7.40-7.29 (3H, m), 2.92-2.85 (3H, m), 2.21-2.17 (1H, m), 1.98-1.93 (1H, m), 1.82-1.63 (6H, m), 1.43-1.26 (6H, m), 0.92 (3H, t, J=7.2 Hz). ¹³C NMR (100 MHz, CDCl₃): δ 200.1, 177.8, 149.0, 140.1, 139.2, 137.9, 134.3, 132.6, 132.0, 130.6, 128.9, 127.4, 125.2, 124.3, 49.1, 38.6, 32.6, 30.2, 30.0, 26.6, 26.4, 26.1, 22.6, 14.1.

### Example 56

### General Procedure for Iron-Catalyzed Alkyl-Imidoyl Chloride Cross-Coupling

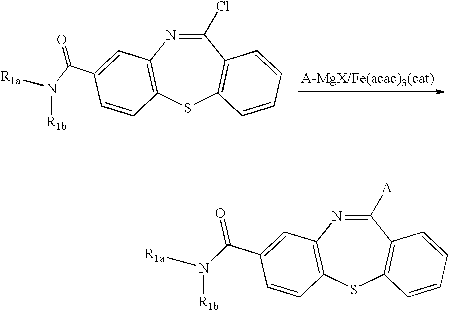

A flame-dried flask was charged under argon with imidoyl chloride amide (0.05 mmol), Fe(acac)₃ (0.9 mg, 0.0025 mmol), THF (1 mL) and NMP (0.1 mL). A solution of alkylmagnesium halogen (2M in Et₂O, 100 μL, 0.20 mmol) was slowly added to the resulting red solution, causing an immediate color change to dark brown. The resulting mixture was stirred for 10 min, and the reaction was then carefully quenched with NH₄Cl (aq) and diluted with Et₂O. The organic phase was washed with brine, dried (Na₂SO₄), filtered and evaporated to give the crude product. Purification by column chromatography (ethyl acetate/heptane/MeOH 1:1:0.05) gave the following compounds (60-90%).

### Example 57

### (11-Butyl-dibenzo[b,f][1,4]thiazepin-8-yl)-[4-(2,4-Dimethyl-phenyl)-piperazin-1-yl]methanone.

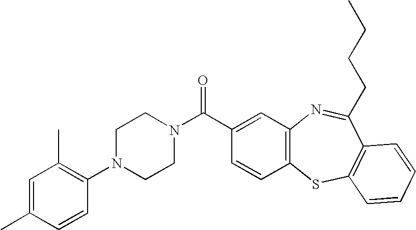

The reaction was performed according to the general procedure, which gave 18.7 mg (77%) of the title compound. ¹H NMR (400 MHz, CDCl₃) δ 7.45 (m, 2H), 7.40-7.32 (m, 3H), 7.23 (d, 1H, J=1.8 Hz), 7.08 (dd, 1H,J=8.0, 1.8 Hz), 7.02 (br s, 1H), 6.98 (br d, 1H, J=8.0 Hz), 6.89 (d, 1H, J=8.0 Hz), 3.88 (br s, 2H), 3.58 (br s, 2H), 3.05-2.75 (m, 6H), 2.29 (s, 6H), 1.7 (m, 2H), 1.5 (m, 2H), 0.95 (t, 3H, J=7.4 Hz); ¹³C NMR (100 MHz, CDCl₃) δ 174.6, 169.7, 149.1, 148.6, 140.0, 139.0, 137.0, 133.5, 132.9, 132.8, 132.1, 130.8, 130.6, 128.8, 127.9, 127.4, 123.8, 123.8, 119.4, 42.3, 29.6, 22.7, 20.9, 17.8, 14.2; MS (ES⁺, M+1)=484.

### Example 58

### [4-(2,4-Dimethyl-phenyl)-piperazin-1-yl]-(11-pentyl-dibenzo[b,f][1,4]thiazepin-8-yl)methanone

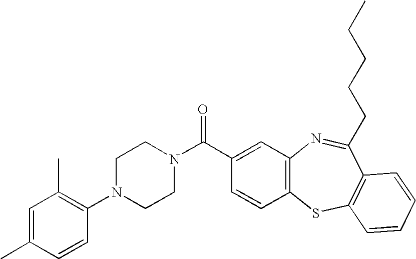

The reaction was performed according to the general procedure, which gave 20.1 mg 81%) of the title compound. ¹H NMR (400 MHz, CDCl₃) δ 7.46 (m, 2H), 7.40-7.32 (m, 3H), 7.23 (d, 1H, J=1.6 Hz), 7.08 (dd, 1H, J=8.0, 1.8 Hz), 7.02 (br s, 1H), 6.98 (br d, 1H, J=8.0 Hz), 6.89 (d, 1H, J=8.0 Hz), 3.88 (br s, 2H), 3.58 (br s, 2H), 3.05-2.75 (m, 6H), 2.29 (s, 6H), 1.7 (m, 2H), 1.5-1.2 (m, 4H), 0.95 (t, 3H, J=7.0 Hz); ¹³C NMR (100 MHz, CDCl₃) δ 174.9, 170.0, 149.3, 148.9, 140.3, 139.3, 137.3, 133.8, 133.1, 133.1, 132.4, 131.1, 130.9, 129.0, 128.2, 127.6, 124.1, 119.7, 42.7, 32.0, 27.3, 22.9, 21.2, 18.1, 14.5; MS (ES⁺, M+1)=498.

### Example 59

### [4-(2,4-Dimethyl-phenyl)-piperazin-1-yl]-(11-isobutyl-dibenzo[b,f][1,4]thiazepin-8-yl)methanone

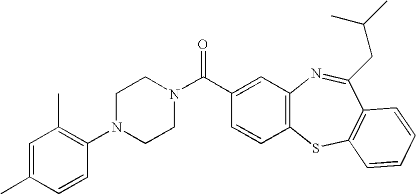

The reaction was performed according to the general procedure, which gave 17.3 mg (72%) of the title compound. MS (ES⁺, M+1)=484.

### Example 60

### (11-Cyclohexyl-dibenzo[b,f][1,4]thiazepin-8-yl)-[4-(2,4-dimethyl-phenyl)-piperazin-1-yl]methanone

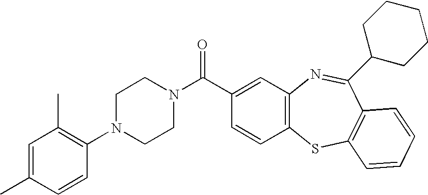

The reaction was performed according to the general procedure, which gave 16.8 mg (66%) of the title compound. MS (ES⁺, M+1)=510

### Example 61

### [11-(4-chloro-phenyl)-dibenzo[b,f][1,4]thiazepin-8-yl)]-[4-(2,4-dimethyl-phenyl)-piperzin-1-yl]-methanone

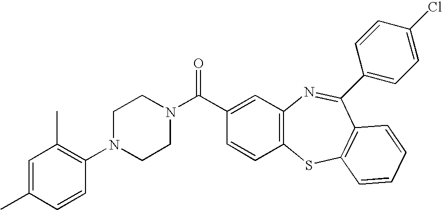

The reaction was performed according to the general procedure, which gave 16.2 mg (60%) of the title compound. MS (ES⁺, M)=538.

### Example 62

### 11-Propyl-dibenzo[b,f][1,4]thiazepine-8-carboxylic acid piperidin-1-ylamide

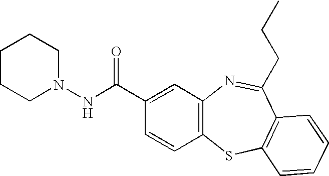

The reaction was performed according to the general procedure, which gave 15.3 mg (81%) of the title compound. MS (ES⁺, M+1)=380.

### Example 63

### 11-Butyl-dibenzo[b,f][1,4]thiazepine-8-carboxylic acid piperidin-1-ylamide

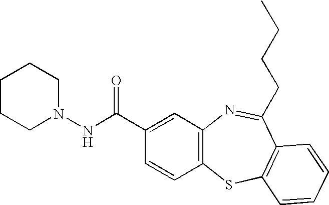

The reaction was performed according to the general procedure, which gave 15.8 mg (80%) of the title compound. MS (ES⁺, M+1)=394.

### Example 64

### 11-Pentyl-dibenzo[b,f][1,4]thiazeipine-8-carboxylic acid piperidin-1-ylamide

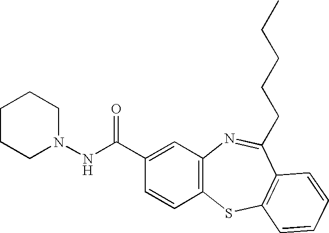

The reaction was performed according to the general procedure, which gave 16.1 mg (79%) of the title compound. MS (ES⁺, M+1)=408.

### Example 65

### 11-Isobutyl-dibenzo[b,f][1,4]thiazepine-8-carboxylic acid piperidin-1-ylamide

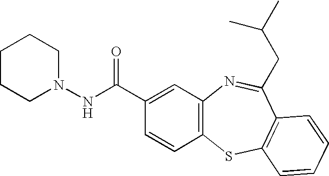

The reaction was performed according to the general procedure, which gave 16.2 mg (82%) of the title compound. MS (ES⁺, M+1)=394.

### Example 66

### 11-Cyclohexyl-dibenzo[b,f][1,4]thiazepine-8-carboxylic acid piperidin-1-ylamide

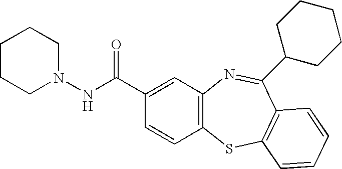

The reaction was performed according to the general procedure, which gave 15.9 mg (76%) of the title compound. MS (ES⁺, M+1)=420.

### Example 67

### 4-[(11-Propyl-dibenzo[b,f][1,4]thiazepine-8-carbonyl)-amino]-piperidine-1-carboxylic acid ethyl ester

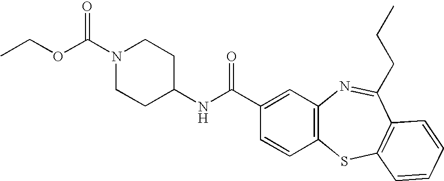

The reaction was performed according to the general procedure, which gave 19.7 mg (87%) of the title compound. MS (ES⁺, M+1)=452.

### Example 68

### 4-[(11-Butyl-dibenzo[b,f][1,4]thiazepine-8-carbonyl)-amino]-piperidine-1-carboxylic acid ethyl ester

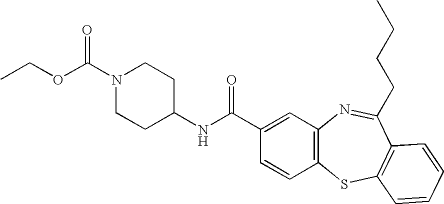

The reaction was performed according to the general procedure, which gave 19.2 mg (83%) of the title compound. ¹H NMR (400 MHz, CD30D) δ 7.45 (dd, 1H, J=1.4, 0.8 Hz), 7.44-7.37 (m, 3H), 7.34-7.28 (m, 3H), 4.03 (q, 2H, J=7.1 Hz), 4.03 (m, 2H), 3.92 (m, 1H), 3.00 (m, 1H), 2.84 (br t, 2H, J=11.9), 2.78 (m, 1H), 1.80 (d, 2H, J=12.5 Hz), 1.52 (m, 2H), 1.37 (m, 4H), 1.15 (t, 3H, J=7.0 Hz), 0.83 (t, 3H, J=7.4 Hz); MS (ES⁺, M+1)=466.

### Example 69

### 4-[(11-Pentyl-dibenzo[b,f][1,4]thiazepine-8-carbonyl)-amino]-piperidine-1-carboxylic acid ethyl ester

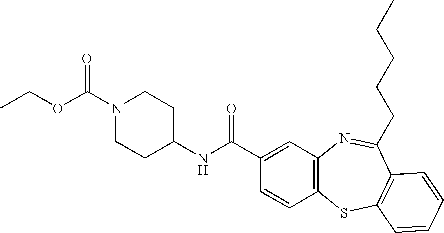

The reaction was performed according to the general procedure, which gave 20.1 mg (84%) of the title compound. ¹H NMR (400 MHz, CDCl₃) δ 7.46 (m, 4H), 7.39-7.32 (m, 3H), 5.89 (d, 1H, J=7.6 Hz), 4.12 (q, 2H, J=7.0 Hz), 4.10 (m, 3H), 2.92 (m, 4H), 1.98 (d, 2H, J=11.9 Hz), 1.68 (m, 2H), 1.39 (m, 6H), 1.25 (t, 3H, J=7.1 Hz), 0.90 (t, 3H, J=7.2 Hz); ¹³C NMR (100 MHz, CDCl₃) δ 174.6, 165.9, 155.4, 148.6, 139.6, 138.7, 135.3, 132.6, 132.0, 130.7, 128.6, 127.6, 123.9, 123.0, 61.4, 47.1, 42.7, 42.2, 32.0, 31.4, 26.8, 22.4, 14.6, 13.9; MS (ES⁺, M+1)=480.

### Example 70

### 4-[(11-Isobutyl-dibenzo[b,f][1,4]thiazepine-8-carbonyl)-amino]-piperidine-1-carboxylic acid ethyl ester

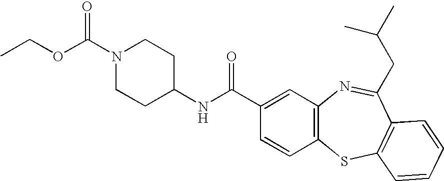

The reaction was performed according to the general procedure, which gave 17.3 mg (74%) of the title compound. ¹H NMR (400 MHz, CDCl₃) δ 7.46 (m, 4H), 7.39-7.31 (m, 3H), 5.98 (d, 1H, J=7.8 Hz), 4.12 (q, 2H, J=7.0 Hz), 4.10 (m, 3H), 3.03 (dd, 1H, J=14.1, 5.5 Hz), 2.85 (t, 2H, J=13.7 Hz), 2.63 (dd, 1H, J=14.1, 9.0 Hz), 1.98 (m, 3H), 1.35 (m, 2H), 1.25 (t, 3H, J=7.1 Hz), 1.08 (d, 3H, J=6.5 Hz), 1.03 (d, 3H, J=6.5 Hz); ¹³C NMR (100 MHz, CDCl₃) δ 174.2, 166.2, 155.7, 148.8, 139.8, 139.0, 135.5, 132.9, 132.8, 132.4, 131.0, 128.9, 128.1, 124.3, 123.4, 61.6, 51.7, 47.4, 43.0, 42.2, 32.3, 27.3, 23.4, 22.4, 14.9; MS (ES⁺, M+1)=466.

### Example 71

### 4-[(11-Cyclohexyl-dibenzo[b,f][1,4]thiazepine-8-carbonyl)-amino]-piperidine-1-carboxylic acid ethyl ester

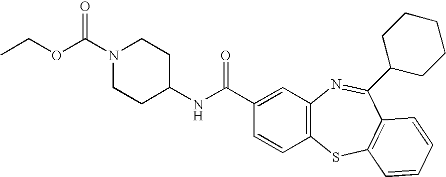

The reaction was performed according to the general procedure, which gave 21.3 mg (87%) of the title compound. MS (ES⁺, M+1)=492

### Example 72

### 4-[(11-(4-chloro-phenyl)-dibenzo[b,f][1,4]thiazepine-8-carbonyl)-amino]-piperidine-1-carboxylic acid ethyl ester

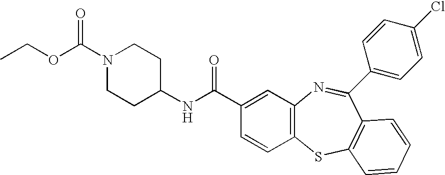

The reaction was performed according to the general procedure, which gave 18.2 mg (70%) of the title compound. MS (ES⁺, M)=520

Synthesis of a Carbon Analogue

### Example 73

### 4-(2-Methoxycarbonyl-benzyl)-3-nitro-benzoic acid ethyl ester (xx1)

A solution of methyl 2-(bromomethyl)benzoate (261 mg, 1.14 mmol) and tetrakis(triphenylphosphine)palladium(0) (52 mg, 0.045 mmol) in DME (2 mL) under argon was stirred at room temperature for 10 min. 4-Ethoxycarbonyl-2-nitrophenylboronic acid (308 mg, 1.29 mmol) dissolved in DME/EtOH 2:1 (3 mL) was added followed by 2M aq. Na₂CO₃ (2 mL) and stirring was continued for 2 h. The reaction mixture was concentrated in vacuo and purified by column chromatography using EtOAc (0-10%) in heptane as the eluent furnishing 338 mg of xx1 as a colourless solid (1.13 mmol, 65%). ¹H NMR (400 MHz, CDCl₃): 8.58 (d, 2H), 8.06 (dd, 1H), 8.02 (dd, 2H), 7.50 (dt, 1H), 7.38 (dt, 1H), 7.18 (d, 1H), 7.06 (d, 1H), 4.69 (s, 2H), 4.39 (q, 2H), 3.76 (s, 3H), 1.40 (t, 3H).

### Example 74

### 4-(2-Carboxy-benzyl)-3-nitro-benzoic acid (xx2)

A solution of xx1 (159 mg, 0.46 mmol) in THF (14 mL) and 1M aq. LiOH (4.6 mL, 4.6 mmol) was stirred at 60° C. for 2 h, then allowed to cool to room temperature. THF was removed at reduced pressure and the resulting aqueous mixture was treated with 2M HCl until the pH was about 1. Filtration provided 93 mg (0.3 mmol, 67%) of xx2 as a yellow solid. ¹H NMR (400 MHz, CD₃OD): 8.49 (d, 1H), 8.06 (dd, 1H), 8.02 (dd, 1H), 7.53 (dt, 1H), 7.40 (dt, 1H), 7.26 (d, 1H), 7.12 (d, 1H), 4.69 (s, 2H).

### Example 75

### 3-Amino-4-(2-carboxy-benzyl)-benzoic acid (xx3)

A solution of xx2 (79 mg, 0.26 mmol) in MeOH (3 mL) containing PtO₂ (6 mg) and Pd/C (7 mg) was stirred under a hydrogen atmosphere for 2 h at room temperature. Filtration and concentration in vacuo provided 71 mg (0.267 mmol, 100%) of xx3 as yellow oil. ¹H NMR (400 MHz, CD₃OD): 7.26 (dd, 1H), 7.44-7.38 (m, 2H), 7.32-7.26 (m, 2H), 7.16 (d, 1H), 6.87 (d, 1H), 4.29 (s, 2H).

### Example 76

### 6-Oxo-6,11-dihydro-5H-dibenzo[b,e]azepine-3-carboxylic acid (xx4)

To a stirred solution of xx3 (70 mg, 0.26 mmol) in THF (3 mL) at room temperature was added carbonyldiimidazole (167 mg, 1.03 mmol) in small portions and stirring was continued. After 4 h, 4M HCl (3 mL) was added followed by water. Filtration and drying provided 51 mg (0.2 mmol, 78%) of xx4 as a colourless solid. The product was further purified by crystallation from 2-propanol. ¹H NMR (400 MHz, DMSO-d₆): 10.58 (s, 1H), 7.70-7.61 (m, 3H), 7.48-7.30 (m, 4H), 3.95 (s, 2H).

### Example 77

### 6-chloro-11H-dibenzo[b,e]azepine-3-carboxylic acid piperidin-1-ylamide

A solution of 6-oxy-5,6-dihydro-11H-dibenzo[b,e]azepine-3-carboxylic acid (45 mg, 0.18 mmol) and phosphorus pentachloride (187 mg, 0.9 mmol) in 2 mL toluene was heated to 90° C. for 6 h. Toluene and excess of phosphorus pentachloride were removed at reduced pressure to give 60 mg of 6-chloro-11H-dibenzo[b,e]azepine-3-carbonyl chloride. 1-Aminopiperidine (0.078 ml, 0.7 mmol) dissolved in CH₂Cl₂ was added to the crude acid chloride dissolved in CH₂Cl₂ at room temperature. EtOAc and H₂O were added to the reaction mixture after 1 h. The H₂O phase was extracted once with EtOAc and the combined organic phases were washed with saturated aqueous NaHCO₃ and brine and dried (Na₂SO₄). Filtration and concentration at reduced pressure of the organic phase followed by purification of the crude product by column chromatography (heptane-EtOAc 1:1) gave 25 mg (40%) of the title compound. ¹H NMR (400 MHz, CDCl₃) δ 7.81 (d, 2H, J=7.4 Hz), 7.68 (dd, 1H, J=8.0, 1.8 Hz), 7.59 (s, 1H), 7.47 (dt, 1H, J=7.4, 1.2 Hz), 7.33 (t, 1H, J=7.6 Hz), 7.27 (t, 1H, J=7.4 Hz), 3.74 (s, 2H), 2.83 (m, 4H), 1.72 (m, 4H), 1.42 (m, 2H); MS (ES⁺, M+1)=354.

### Example 78

### 6-cyclohexyl-11H-dibenzo[b,e]azepine-3-carboxylic acid piperidin-1-ylamide

The reaction was performed according to the general procedure for iron-catalyzed alkyl-imidoyl chloride cross coupling using 25 mg of X and an excess (0.35 ml) of cyclohexylmagnesium chloride (2M). This gave 13.7 mg (49%) of the title compound. MS (ES⁺, M+1)=402; UV/MS purity 100/100.

Synthesis of an Oxygen Analogue

### Example 79

### 4-(2-Methoxycarbonyl-phenoxy)-3-nitro-benzoic acid ethyl ester (xx5)

To a stirred solution of ethyl 4-flouro-3-nitrobenzoate (2.53 g, 11.87 mmol) in DMF (40 mL) containing Cs₂CO₃ (4.26 g, 13.06 mmol) at 100° C. was added drop wise methyl salicylate (1.69 mL, 13.06 mol) dissolved in DMF (40 mL) over 2 h. After 15 min the reaction mixture was allowed to reach room temperature and then diluted with EtOAc (100 mL) and washed with water (2×100 mL). The aqueous layer was extracted with DCM (100 mL). Drying (MgSO₄) of the combined organic layers followed by filtration, concentration in vacuo and purification by CC using EtOAc (0-40%) in heptane provided 3.75 g (10.85 mmol, 91%) of xx5 as a yellow solid. ¹H NMR (400 MHz, CDCl₃): 8.60 (d, 1H), 8.04 (dt, 2H), 7.62 (dt, 1H), 7.38 (dt, 1H), 7.19 (dd, 1H), 6.73 (d, 1H), 4.37 (q, 2H), 3.71 (s, 3H), 1.38 (t, 3H).

### Example 80

### 4-(2-Carboxy-phenoxy)-3-nitro-benzoic acid (xx6)

A solution of xx5 (3.68 gmg, 10.65 mmol) in THF (200 mL) and 1M aq. LiOH (100 mL, 100 mmol) was stirred at 60° C. for 2 h, then allowed to cool to room temperature. THF was removed at reduced pressure and the resulting aqueous mixture was treated with 2M HCl until the pH was about 1. Filtration provided 2.75 g (9.08 mmol, 85%) of xx6 as a pale yellow solid. ¹H NMR (400 MHz, CD₃OD): 8.53 (d, 1H), 8.10 (dd, 1H), 8.04 (dd, 1H), 7.69 (dt, 1H), 7.42 (dt, 1H), 7.26 (dd, 1H), 6.82 (d, 1H).

### Example 81

### 3-Amino-4-(2-carboxy-phenoxy)-benzoic acid (xx7)

A solution of xx6 (2.75 g, 9.08 mmol) in MeOH (80 mL) containing PtO₂ (59 mg) and Pd/C (211 mg) was stirred for 2 h under a hydrogen atmosphere at room temperature. Filtration and concentration in vacuo provided 2.47 g (9.05 mmol, 100%) of xx7 as a pale yellow solid. ¹H NMR (400 MHz, CD₃OD): 7.89 (dd, 1H), 7.54-7.47 (m, 2H), 7.31 (dt, 1H), 7.21 (dt, 1H), 6.97 (d, 1H), 6.68 (d, 1H).

### Example 82

### 11-Oxo-10,11-dihydro-dibenzo[b,f][1,4]oxazepine-8-carboxylic acid (xx8)

To a stirred solution of xx7 (2.44 g, 0.26 mmol) in THF (100 mL) at room temperature was added carbonyldiimidazole (3.7 g, 22.8 mmol) in small portions and stirring was continued. After 4 h 4M HCl (100 mL) was added followed by copious amounts of water. Filtration and drying followed by crystallization (2-propanol) provided 1.017 g (3.99 mmol, 45%) of xx8 as white crystals. ¹H NMR (400 MHz, DMSO-d₆): 10.61(s, 1H), 7.77-7.74 (m, 2H), 7.67 (dd, 1H), 7.60 (dt, 1H), 7.39 (d, 1H), 7.34 (d, 1H) 7.31 (dt, 1H).

### Example 83

### 11-Chloro-dibenzo[b,f][1,4]oxazepine-8-carboxylic acid piperidin-1-ylamide (xx9)

To a stirred solution of xx8 (476 mg, 1.86 mmol) in toluene (20 mL) and thionyl chloride (20 mL) was added DMF (0.5 mL) and stirring was continued at 80° C. for 19 h. The reaction mixture was concentrated in vacuo and re-dissolved in anhydrous DCM (20 (mL) and added to a solution of 1-aminopiperidine (604 μL, 5.59 mmol) dissolved in DCM (20 mL) at 0° C. and stirring was continued for 2 h. The resulting reaction mixture was concentrated in vacuo and purified by CC using EtOAc (0-70%) in heptane affording 353 mg (0.99 mmol, 53%) of xx9 as a pale yellow solid. ¹H NMR (400 MHz, CDCl₃): 7.77-7.72 (m, 2H), 7.63 (s, 1H), 7.53 (dt, 1H), 7.22 (dt, 1H), 7.18 (dd, 1H), 2.92 (br s), 1.76 (br s), 1.43 (br s).

### Example 84

### 11-Cyclohexyl-dibenzo[b,f][1,4]oxazepine-8-carboxylic acid piperidin-1-ylamide (xx10)

To a flame dried flask loaded with Fe(acac)₃ under argon was added sequentially xx9 (79 mg, 0.22 mmol) dissolved in dry THF, NMP (0.5 mL) and a 2M etheral solution of cyclohexylmagnesium chloride (440 μL, 0.88 mmol) at −78° C. and the reaction mixture was allowed to slowly reach ambient temperature. After additionally 2 h sat aq NH₄Cl (5 mL) was added followed by EtOAc (10 mL). After separation of the layers, the aq layer was extracted with EtOAc (2×10 mL). The combined organic layers were dried (MgSO₄), filtered, concentrated in vacuo and purified by CC using EtOAc (0-50%) in heptane as the eluent affording 89 mg (0.22 mmol, 100%) of xx10 as a grey solid. ¹H NMR (400 MHz, CDCl₃): 7.65 (br s, 1H), 7.63 (br s, 1H), 7.45-7.39 (m, 2H), 7.21 (dt, 1H), 7.15 (dd, 2H), 3.10 (br s), 2.91 (tt), 1.97 (d), 1.85 (br s), 1.74 (d), 1.61 (dd), 1.50 (br s), 1.42-1.29 (m), 1.25 (br s), 0.89-0.85 (m).

### Example 85

### Library Synthesis: Formation of Amidoimidoyl Chlorides

The amidoimidoyl chlorides were synthesized according to the general procedure for amide formation at 0.5 mmol scale except that the reaction mixture was passed through a pad of acidic alumina oxide and eluted with a mixture of CH₂Cl₂ and EtOAc. The eluents were concentrated at reduced pressure and the obtained crude products were directly used in the next reactions without further purifications or characterization.

### Example 86

### 11-(chloro)-dibenzo[b,f][1,4]thiazepin-8-yl-(piperidin-1-yl)-methanone

173 mg

### Example 87

### N-benzyl-11-(chloro)-dibenzo[b,f][1,4]thiazepine-8-carboxamide

148 mg

### Example 88

### N-(1-phenylethyl)-11-(chloro)-dibenzo[b,f][1,4]thiazepine-8-carboxamide

168 mg

### Example 89

### N-(butyl)-11-(chloro)-dibenzo[b,f][1,4]thiazepine-8-carboxamide

138 mg

### Example 90

### N-(3-phenylpropyl)-11-(chloro)-dibenzo[b,f][1,4]thiazepine-8-carboxamide

167 mg

### Example 91

### N-(2-phenylethyl)-11-(chloro)-dibenzo[b,f][1,4]thiazepine-8-carboxamide

160 mg

### Example 92

### N-(2-chlorobenzyl)-11-(chloro)-dibenzo[b,f][1,4]thiazepine-8-carboxamide

161 mg

### Example 93

### N-(2,4-dichlorobenzyl)-11-(chloro)-dibenzo[b,f][1,4]thiazepine-8-carboxamide

120 mg

### Example 94

### N-(2-(4-chlorophenyl)ethyl)-11-(chloro)-dibenzo[b,f][1,4]thiazepine-8-carboxamide

167 mg

### Example 95

### N-(2-(3-chlorophenyl)ethyl)-11-(chloro)-dibenzo[b,f][1,4]thiazepine-8-carboxamide

171 mg

### Example 96

### N-(3-chlorobenzyl)-11-(chloro)-dibenzo[b,f][1,4]thiazepine-8-carboxamide

176 mg

### Example 97

### N-(2-bromobenzyl)-11-(chloro)-dibenzo[b,f][1,4]thiazepine-8-carboxamide

180 mg

### Example 98

### N-(2-phenyl-propyl)-11-(chloro)-dibenzo[b,f][1,4]thiazepine-8-carboxamide

172 mg

### Example 99

### N-((N-ethyl-N-phenyl)aminoethyl)-11-(chloro)-dibenzo[b,f][1,4]thiazepine-8-carboxamide

168 mg

### Example 100

### 11-(chloro)-dibenzo[b,f][1,4]thiazepin-8-carboxylic acid morpholin-4-yl amide

160 mg

### Example 101

### N-(4-fluorobenzyl)-11-(chloro)-dibenzo[b,f][1,4]thiazepine-8-carboxamide

120 mg

Series B

The following compounds were prepared according to the general procedure for an iron-catalyzed alkyl-imidoyl chloride cross-coupling starting from the appropriate imidoylchloride (15 mg) and cyclohexylmagnesium chloride (6 eq). When the reactions were completed saturated ammonium chloride (1 ml) and EtOAc (2 ml) were added to the reaction mixtures. The organic phases were passed through a short silica column (eluted with EtOAc). After concentration at reduced pressure, the obtained crude products were purified by preparative HPLC.

### Example 102

### 11-(cyclohexyl)-dibenzo[b,f][1,4]thiazepin-8-yl-(piperidin-1-yl)-methanone

0.6 mg, UV/MS purity 90/90

### Example 103

### N-benzyl-11-(cyclohexyl)-dibenzo[b,f][1,4]thiazepine-8-carboxamide

5.1 mg, UV/MS purity 98/83

### Example 104

### N-(1-phenylethyl)-11-(cyclohexyl)-dibenzo[b,f][1,4]thiazepine-8-carboxamide

2.2 mg, UV/MS purity 98/87

### Example 105

### N-(butyl)-11-(cyclohexyl)-dibenzo[b,f][1,4]thiazepine-8-carboxamide

4.6 mg, UV/MS purity 98/91

### Example 106

### N-(2-chlorobenzyl)-11-(cyclohexyl)-dibenzo[b,f][1,4]thiazepine-8-carboxamide

4.5 mg, UV/MS purity 99/85

### Example 107

### N-(2,4-dichlorobenzyl)-11-(cyclohexyl)-dibenzo[b,f][1,4]thiazepine-8-carboxamide

1.8 mg, UV/MS purity 100/82

### Example 108

### N-(2-(4-chlorophenyl)ethyl)-11-(cyclohexyl)-dibenzo[b,f][1,4]thiazepine-8-carboxamide

5.9 mg, UV/MS purity 100/87

### Example 109

### N-(2-(3-chlorophenyl)ethyl)-11-(cyclohexyl)-dibenzo[b,f][1,4]thiazepine-8-carboxamide

6.6 mg, UV/MS purity 99/90

### Example 110

### N-(3-chlorobenzyl)-11-(cyclohexyl)-dibenzo[b,f][1,4]thiazepine-8-carboxamide

4.8 mg, UV/MS purity 99/87

### Example 111

### N-(2-bromobenzyl)-11-(cyclohexyl)-dibenzo[b,f][1,4]thiazepine-8-carboxamide

0.8 mg, UV/MS purity 100/83

### Example 112

### N-(2-phenyl-propyl)-11-(cyclohexyl)-dibenzo[b,f][1,4]thiazepine-8-carboxamide

5.3 mg, UV/MS purity 93/83

### Example 113

### N-((N-ethyl-N-phenyl)aminoethyl)-11-(cyclohexyl)-dibenzo[b,f][1,4]thiazepine-8-carboxamide

3.2 mg, UV/MS purity 98/79

### Example 114

### 11-(cyclohexyl)-dibenzo[b,f][1,4]thiazepin-8-carboxylic acid morpholin-4-yl amide

3.8 mg, UV/MS purity 96/75

### Example 115

### N-(4-fluorobenzyl)-11-(cyclohexyl)-dibenzo[b,f][1,4]thiazepine-8-carboxamide

3.6 mg, UV/MS purity 98/74

### Example 116

### Typical Procedures for the Palladium-catalyzed Cross-coupling Reaction of Imidoyl Chlorides

Method A: The organozinc bromide (0.40 mmol) was added to a dried, argon-flushed 7 ml flask charged with the imidoyl chloride (0.20 mmol) in dry NMP (0.25 ml), Pd₂(dba)₃ (0.01 mmol) and tri-2-furylphosphine (0.04 mmol). The resulting mixture was heated in microwave at 100° C. for 5 min., and then cooled to room temperature. The reaction was quenched with saturated aqueous NH₄Cl and extracted twice with ether. The combined organic phases were washed with water, brine, dried (Na₂SO₄). Filtration and removal of the solvent at reduced pressure gave the crude product, which was purified by either column chromatography, ion exchange extraction using a Varian Bond Elut® SCX column or both. The SCX column was pre-washed twice with MeOH (10 ml). The crude product was dissolved in EtOAc and applied to the column. The column was washed twice with MeOH. The product was eluated using ammonia (10% in MeOH).

Method B: The organozinc bromide (0.48 mmol) was added to a dried, argon-flushed 4 ml flask charged with the imidoyl chloride (0.24 mmol) in dry THF (2.0 ml) and Pd(PPh₃)₄ (0.012 mol; on resin bead). The resulting mixture was shaken at room temperature for 2 hours, and the reaction was quenched with saturated aqueous NH₄Cl and extracted twice with ether. The combined organic phases were washed with water, brine, dried (Na₂SO₄). Filtration and removal of the solvent at reduced pressure gave the crude product.

### Example 117

### 4-(8-chloro-5H-dibenzo[b,f][1,4]diazepine-11-yl)-butyronitrile

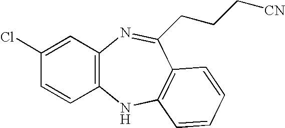

The reaction was performed according to the typical procedure (Palladium Method A) using 8,11-dichloro-5H-dibenzo[b,e][1,4]diazepine (53 mg, 0.20 mmol). Purification by column chromatography (EtOAc/Heptane 1:4) and SCX afforded 24 mg (41%) of the title compound as an oil. ¹H NMR (400 MHz, CDCl₃) δ 7.30-7.24 (m, 2H), 7.12 (t, 1H, J=2.4 Hz), 7.12-6.95 (m, 2H), 6.70-6.68 (m, 1H), 6.58 (dd, 1H, J=8.0, 2.0 Hz), 4.89 (br s, 1H), 2.96-2.92 (m, 2H), 2.59-2.55 (m, 2H), 2.17-2.09 (m, 2H); ¹³C NMR (100 MHz, CDCl₃) δ 171.6, 153.6, 141.7, 141.5, 132.3, 129.4, 129.0, 128.6, 128.0, 126.5, 123.7, 120.8, 120.0, 119.9, 38.5, 22.6, 16.7.

### Example 118

### 8-chloro-11-cyclohexyl-5H-dibenzo[b,e][1,4]diazepine

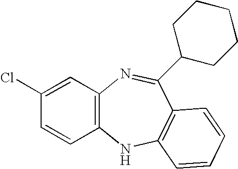

The reaction was performed according to the typical procedure (Palladium Method A) using 8,11-dichloro-5H-dibenzo[b,e][1,4]diazepine (53 mg, 0.20 mmol. Purification by column chromatography (ethyl acetate/heptane 1:4) afforded 43 mg (65%) of the title compound as an oil. ¹H NMR (400 MHz, CDCl₃) δ 7.32 (dd, 1H, J=7.6, 1.2 Hz), 7.26-7.21 (m, 1H), 7.17 (d, 1H, J=2.4 Hz), 7.03-6.99 (m, 1H), 6.92 (dd, 1H, J=8.4, 2.4), 6.70-6.67 (m, 1H), 6.58 (d, 1H, J=8.4 Hz), 4.83 (1H, bs), 2.78 (1H, m), 1.95-1.90 (m, 2H), 1.87-1.83 (m, 2H), 1.74-1.70 (m, 1H), 1.64-1.54 (m, 2H), 1.40-1.26 (m, 3H); ¹³C NMR (100 MHz, CDCl₃) δ 154.0, 142.2, 141.6, 131.6, 129.2(2), 128.7(3), 125.8, 123.5, 120.5, 119.6, 47.9, 31.8, 26.7, 26.4.

### Example 119

### 11-butyl-dibenzo[b,f][1,4]oxazepine (40)

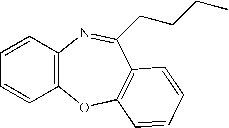

1): The reaction was performed according to the typical procedure (Palladium Method B) using 11-chloro-dibenzo[b,f][1,4]oxazepine (54 mg, 0.24 mmol). Purification by column chromatography (ethyl acetate/heptane 1:4) afforded 18 mg (30%) of the title compound as an oil. ¹H NMR (400 MHz, CDCl₃) δ 7.44-7.41 (m, 2H), 7.30-7.26 (m, 1H), 7.22-7.18 (m, 2H), 7.16-7.13 (m, 3H), 2.93 (t, 2H, J=7.2 Hz), 1.71 (quintet, 2H, J=7.6 Hz), 1.46 (sextet, 2H), 0.99 (t, 3H, J=7.2 Hz); ¹³C NMR (100 MHz, CDCl₃) δ 171.2, 161.7, 152.8, 141.0, 132.7, 128.7, 128.4, 127.9, 127.2, 125.7, 125.3, 121.1, 120.8, 40.2, 29.9, 22.7, 14.2.

2): The reaction was performed according to the typical procedure (Palladium Method B) using 11-chloro-dibenzo[b,f][1,4]oxazepine (48 mg, 0.21 mmol) except that Pd(P(t-Bu)₃)₂ (6 mg, 0.024 mmol) was used as catalyst and the reaction time (0.5 h) was shortened. ¹H-NMR yield based on anisole as an internal standard showed the title compound in 44% yield.

3): The reaction was performed according to the typical procedure (Palladium Method B) using 11-chloro-dibenzo[b,f][1,4]oxazepine (49 mg, 0.21 mmol) except that PdCl₂(PPh₃)₂ (15 mg, 0.021 mmol) was used as catalyst and the reaction time (1 h) was shortened. ¹H-NMR yield based on anisole as an internal standard showed the title compound in 50% yield.

Typical Procedure Copper-catalyzed Cross-coupling Reaction of Imidoyl Chlorides with Alkylmagnesium Halides

A flame dried 10 ml flask was charged under argon with the imidoyl chloride (47 mg, 0.21 mmol) and CuCN (2 mg, 0.021 mmol) in dry THF (2 ml) and NMP (0.2 ml). Butylmagnesium chloride (0.20 ml, 0.40 mmol) was slowly added to the solution. The reaction was stirred for 24 hours at room temperature, then quenched with saturated aqueous NH₄Cl and diluted with Et₂O. The organic phase was washed with water, brine and dried (Na₂SO₄). Filtration and removal of the solvent at reduced pressure gave the crude product

### Example 120

### 11-butyl-dibenzo[b,f][1,4]oxazepine

1): The reaction was performed according to the typical procedure using 11-chloro-dibenzo[b,f][1,4]oxazepine (47 mg, 0.21 mmol). The yield of the title compound was determined to be 64% according to ¹H-NMR analysis using toluene as an internal standard.

2): The reaction was performed according to the typical procedure using 11-chloro-dibenzo[b,f][1,4]oxazepine (47 mg, 0.21 mmol) except that CuCl₂ (3 mg, 0.021 mmol) was used in this reaction. The yield of the title compound was determined to be 52% according to ¹H-NMR analysis using toluene as an internal standard.

3): A flame dried 10 ml flask was charged under argon with CuCN:2LiCl (0.42 ml, 0.42 mmol) and butyl magnesium chloride (0.25 ml, 0.42 mmol) was slowly added at room temperature. Fe(acac)₃ (4 mg, 0.001 mmol) was then added and a solution of 11-chloro-dibenzo[b,f][1,4]oxazepine (49 mg, 0.21 mmol) in THF was added to the reaction mixture. After being stirred for 5 min, the reaction was quenched with saturated aqueous NH₄Cl and diluted with Et₂O. The organic phase was washed with water, brine and dried (Na₂SO₄). Filtration and removal of the solvent at reduced pressure gave the crude product. The yield of the title compound was determined to be 96% according to ¹H-NMR analysis using toluene as an internal standard.

4): A flame dried 10 ml flask was charged under argon with the imidoyl chloride 35 (48 mg, 0.21 mmol), MnCl₂ (2.6 mg, 0.021 mmol) in dry THF (2 ml) and NMP (0.20 ml). Butyl magnesium chloride (2 M in Et₂O, 0.25 ml, 0.42 mmol) was then slowly added to the colorless solution, causing an immediate color change to dark brown. The reaction was stirred for 5 min at room temperature, then quenched with saturated aqueous NH₄Cl and diluted with Et₂O. The organic phase was washed with water, brine and dried (Na₂SO₄). Filtration and removal of the solvent at reduced pressure gave the crude product. The yield of 40 was determined to be 94% according to ¹H-NMR analysis using toluene as an internal standard.

A flame dried 10 ml flask was charged under argon with the MnCl₂:2LiCl (0.42 ml, 0.42 mmol) and butyl magnesium chloride (0.25 ml, 0.42 mmol) was slowly added. Fe(acac)₃ (4 mg, 0.001 mmol) was then added and a solution of 11-chloro-dibenzo[b,f][1,4]oxazepine 35 (48 mg, 0.21 mmol) in THF was added to the reaction mixture. After being stirred for 5 min, the reaction was quenched with saturated aqueous NH₄Cl and diluted with Et₂O. The organic phase was washed with water, brine and dried (Na₂SO₄). Filtration and removal of the solvent at reduced pressure gave the crude product. The yield of the title compound was determined to be 95% according to ¹H-NMR analysis using toluene as an internal standard.

Although the invention has been described with reference to the above examples, it will be understood that modifications and variations are encompassed within the spirit and scope of the invention. Accordingly, the invention is limited only by the following claims.

What is claimed is:

1. A process for preparing a compound of formula A-N═C(D)(B), from a compound of formula A-N═C(E)(B) and a compound of formula D-M using an iron catalyst, where the process is represented by Equation (I) 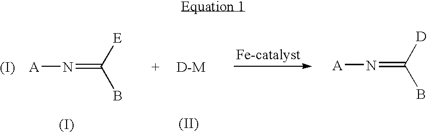
Wherein:
A and B are independently selected from the group consisting of optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁, or A and B taken together, along with the nitrogen atom to which A is attached and the carbon atom to which B is attached, form a ring;
E is selected from the group consisting of halide, sulfonate (—OSO₃R₂), and phosphonate (—OP(O)(OR₂ₐ)(OR_{2b}));
D is selected from group consisting of optionally substituted alkyl, optionally substituted alkenyl, optionally substituted alkynyl, optionally substituted cycloalkyl, optionally substituted cycloalkenyl, optionally substituted aryl, optionally substituted heteroaryl, and optionally substituted heteroalicyclyl;
M is selected from the group consisting of MgY, CaY, ZnY, MnY, and Mg derived metal reagents formed from reaction of MgY and other metal salts, such as Cu(CN)MgCl and Mn(Cl₂)MgCl;
Y is an anionic ligand
R₁, R₁ₐ and R_{1b} are independently selected from the group consisting of hydrogen, optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl;
R₂, R₂ₐ and R_{2b} are independently selected from the group consisting of haloalkyl, optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl; and
Z is O (oxygen) or S (sulfur).

2. The process of claim 1, wherein iron catalyst is selected from the group consisting of finely dispersed metallic iron, FeF₂, FeF₂ 4H₂O, FeF₃ H₂O, FeCl₂, FeCl₂ 4H₂O, FeCl₃, FeCl₃ 6H₂O, FeCl₃(PPh₃), Fe(OEt)₂, Fe(OEt)₃, FeCl₂(PPh₃)₂, FeCl₂(dppe) [dppe=1,2-bis-(diphenylphosphino)ethane], Fe(acac)₂ [acac=acetylacetonate], Fe(acac)₃, tris-(trifluoroacetylacetonato)iron (III), tris-(hexafluoroacetylacetonato)iron (III), tris-(dibenzoylmethido)iron (III), tris-(2,2,6,6-tetramethyl-3,5-diheptanedionate)iron (III), FeBr₂, FeBr₃, FeI₂, Fe(II)acetate, Fe(II)oxalate, Fe(II)stearate, Fe(III)citrate hydrate, Fe(III)pivalate, Fe(II)-D-gluconate 2 H₂O, Fe(OSO₂C₆H₄Me)₃, Fe(OSO₂C₆H₄Me)₃ hydrate, FePO₄, Fe(NO₃)₃, Fe(NO₃)₃ 9 H₂O, Fe(ClO₄)₃ hydrate, FeSO₄, FeSO₄ hydrate, Fe₂(SO₄)₃, Fe₂(SO₄)₃ hydrate, K₃Fe(CN)₆, ferrocene, bis(pentamethylcyclopentadienyl)iron, bis(indenyl)iron, Fe(II)phtalocyanin, Fe(III)phtalocyanin chloride, Fe(CO)₅, Fe(salen)X [salen=N,N-ethylenebis(salicylidenamidato), X=Cl, Br, I], 5,10,15,20-tetraphenyl-21H,23H-porphin-iron(III) halide, 5,10,15,20-tetrakis(pentafluorophenyl)-21H,23H-porphin-iron(III) halide, activated Fe, and iron-magnesium intermetallic compounds.

3. The process of claim 1, in which said organometallic reagent D-M is a Grignard reagent, wherein
D is selected from group consisting of substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heteroalicyclyl; and
M is MgY, wherein Y is fluoride, chloride, bromide, iodide.

4. A process for preparing a compound of Formula IV as shown in Equation 2: 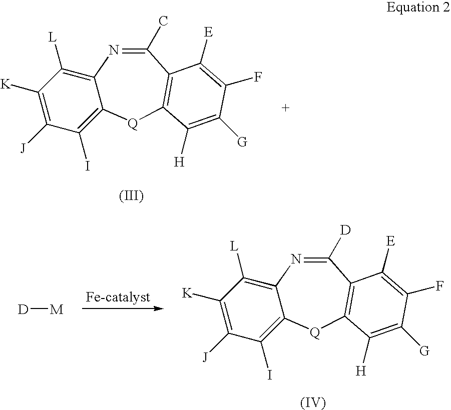
wherein:
C is selected from the group consisting of halide, sulfonate (—OSO₃R₂), and phosphonate (—OP(O)(OR₂ₐ)(OR_{2b}));
D is selected from group consisting of alkyl, alkenyl, alkynyl, cycloalkyl, cycloalkenyl, optionally substituted aryl, optionally substituted heteroaryl, and optionally substituted heteroalicyclyl;
M is MgY;
Y is an anionic ligand;
Q is selected from the group consisting of NR₁, N⁺—O⁻, O, S, S═O, O═S═O, CR₁R₂, C═O, and SiR₁R₂;
E, F, G, H, I, J and L are each independently selected from the group consisting of hydrogen, alkyl, alkenyl, alkynyl, cycloalkyl, cycloalkenyl, optionally substituted aryl, optionally substituted heteroaryl, optionally substituted heteroalicyclyl, halogen, nitro, sulfinyl, sulfonyl, haloalkyl, haloalkoxy, —CN, —C(=Z)R₁, —C(=Z)OR₁, —C(=Z)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;
K is selected from the group consisting of alkyl, alkenyl, alkynyl, cycloalkyl, cycloalkenyl, optionally substituted aryl, optionally substituted heteroaryl, optionally substituted heteroalicyclyl, halogen, hydroxyl, nitro, sulfenyl, sulfinyl, sulfonyl, haloalkyl, haloalkoxy, —CN, —C(=Z)R₁, —C(=Z)OR₁, —C(=Z)NR₁ₐR_{1b}, —C(=Z)N(R₁)NR₁ₐR_{1b}, —C(R₁)═NR₁ₐ, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁;
R₁, R₁ₐ and R_{1b} are independently selected from the group consisting of hydrogen, optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl;
R₂, R₂ₐ and R_{2b} are independently selected from the group consisting of: haloalkyl, optionally substituted alkyl, optionally substituted alkenyl, optionally substituted alkynyl, optionally substituted cycloalkyl, optionally substituted cycloalkenyl, optionally substituted aryl, optionally substituted heteroaryl, optionally substituted heteroalicyclyl; and
Z is O (oxygen) or S (sulfur).

5. The process of claim 4, wherein iron catalyst is selected from the group consisting of finely dispersed metallic iron, FeF₂, FeF₂ 4H₂O, FeF₃ H₂O, FeCl₂, FeCl₂ 4H₂O, FeCl₃, FeCl₃ 6H₂O, FeCl₃(PPh₃), Fe(OEt)₂, Fe(OEt)₃, FeCl₂(PPh₃)₂, FeCl₂(dppe) [dppe=1,2-bis-(diphenylphosphino)ethane], Fe(acac)₂ [acac=acetylacetonate], Fe(acac)₃, tris-(trifluoroacetylacetonato)iron (III), tris-(hexafluoroacetylacetonato)iron (III), tris-(dibenzoylmethido)iron (III), tris-(2,2,6,6-tetramethyl-3,5-diheptanedionate)iron (III), FeBr₂, FeBr₃, FeI₂, Fe(II)acetate, Fe(II)oxalate, Fe(II)stearate, Fe(III)citrate hydrate, Fe(III)pivalate, Fe(II)-D-gluconate 2 H₂O, Fe(OSO₂C₆H₄Me)₃, Fe(OSO₂C₆H₄Me)₃ hydrate, FePO₄, Fe(NO₃)₃, Fe(NO₃)₃ 9 H₂O, Fe(ClO₄)₃ hydrate, FeSO₄, FeSO₄ hydrate, Fe₂(SO₄)₃, Fe₂(SO₄)₃ hydrate, K₃Fe(CN)₆, ferrocene, bis(pentamethylcyclopentadienyl)iron, bis(indenyl)iron, Fe(II)phtalocyanin, Fe(III)phtalocyanin chloride, Fe(CO)₅, Fe(salen)X [salen=N,N-ethylenebis(salicylidenamidato), X=Cl, Br, I], 5,10,15,20-tetraphenyl-21H,23H-porphin-iron(III) halide, 5,10,15,20-tetrakis(pentafluorophenyl)-21H,23H-porphin-iron(III) halide, activated Fe, and iron-magnesium intermetallic compounds.

6. The process of claim 4, in which said organometallic reagent D-M is a Grignard reagent, wherein
D is selected from group consisting of substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heteroalicyclyl; and
M is MgY, wherein Y is fluoride, chloride, bromide, iodide.

7. The process of claim 1 or claim 4, wherein said process is performed in a reaction medium containing one or more solvents selected from the group consisting of ethereal, hydrocarbon, aprotic dipolar, and protic.

8. The process of claim 7, in which said ethereal solvent or hydrocarbon solvent is selected from the group consisting of diethyl ether, tetrahydrofuran, tetrahydropyran, methyl-tetrahydrofuran, 1,4-dioxane, tert-butyl methyl ether, dibutyl ether, di-isopropyl ether, dimethoxyethane, dimethoxymethane, pentane, hexane, heptane, octane, isooctane, cyclohexane, benzene, toluene, xylene, cymene, petrol ether, and decaline.

9. The process of claim 7, wherein said aprotic dipolar solvent is selected from the group consisting of dimethylformamide, dimethylacetamide, dimethylsulfoxide, N-methylpyrrolidinone (NMP); tetramethylurea, sulfolane, diethyl carbonate, 1,3-ethylphosphoric acid triamide (HMPA), N,N,N′,N′-tetramethylethylenediamine (TMEDA).

10. The process of claim 7, wherein said cross coupling reaction is performed in a reaction medium containing one or more ethereal or hydrocarbon solvents selected from the group consisting of diethyl ether, tetrahydrofuran, tetrahydropyran, methyl-tetrahydrofuran, 1,4-dioxane, tert-butyl methyl ether, dibutyl ether, di-isopropyl ether, dimethoxyethane, dimethoxymethane, pentane, hexane, heptane, octane, isooctane, cyclohexane, benzene, toluene, xylene, cymene, petrol ether, decaline, as well as one or aprotic dipolar solvent chosen from: dimethylformamide, dimethylacetamide, dimethylsulfoxide, N-methylpyrrolidinone (NMP); tetramethylurea, sulfolane, diethyl carbonate, 1,3-ethylphosphoric acid triamide (HMPA), and N,N,N′,N′-tetramethylethylenediamine (TMEDA).

11. The process of claim 7, wherein said protic dipolar solvent is selected from the group consisting of water, ethanol, methanol, tert-butanol, isopropanol, and acetic acid

12. The process of claim 7, wherein said cross coupling reaction is performed in a reaction medium containing one or more ethereal or hydrocarbon solvents, selected from the group consisting of diethyl ether, tetrahydrofuran, tetrahydropyran, methyl-tetrahydrofuran, 1,4-dioxane, tert-butyl methyl ether, dibutyl ether, di-isopropyl ether, dimethoxyethane, dimethoxymethane, pentane, hexane, heptane, octane, isooctane, cyclohexane, benzene, toluene, xylene, cymene, petrol ether, decaline, as well as one or aprotic dipolar solvent chosen from: dimethylformamide, dimethylacetamide, dimethylsulfoxide, N-methylpyrrolidinone (NMP); tetramethylurea, sulfolane, diethyl carbonate, 1,3-ethylphosphoric acid triamide (HMPA), N,N,N′,N′-tetramethylethylenediamine (TMEDA) and/or as well as one or protic dipolar solvent chosen from: water, ethanol, methanol, tert-butanol, isopropanol, and acetic acid.

13. The process of claim 1 or claim 4, in which said process is performed in a microwave reactor.
